(19) United States
(12) Patent Application Publication
(10) Pub. No.: US 2018/0008587 A1
(43) Pub. Date: Jan. 11, 2018

(54) THIOHYDANTOIN ANDROGEN RECEPTOR ANTAGONISTS FOR THE TREATMENT OF CANCER

(71) Applicant: Jaressen Pharmaceutica NV, Beerse (BE)

(72) Inventor: Gilles Bignan, Bridgewater, NJ (US)

(21) Appl. No.: 15/644,107

(22) Filed: Jul. 7, 2017

Related U.S. Application Data
(60) Provisional application No. 62/359,987, filed on Jul. 8, 2016.

(51) Int. Cl. A61K 31/4439; A61K 31/4178

(52) U.S. Cl. CPC A61K 31/4439; A61K 31/4178

(57) ABSTRACT

Disclosed are compounds, compositions and methods for treating and/or ameliorating diseases, syndromes, disorders, or conditions associated with AR mutant receptors linked to castration-resistant prostate cancer, in a subject, including a mammal and/or human, in need thereof, who has demonstrated resistance to a first or second generation AR antagonist, comprising, consisting of, and/or consisting essentially of, administering to a subject in need thereof, a therapeutically effective amount of a compound of Formula (I)

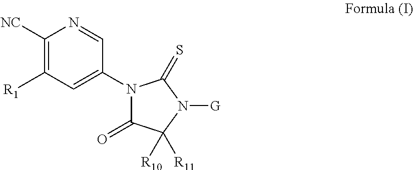

wherein R₁, G, R₁₀, and R₁₁ are defined herein.

### CROSS-REFERENCE TO RELATED APPLICATIONS

The U.S. patent application entitled, Androgen Receptor Modulators and Uses Thereof, U.S. Non Provisional application Ser. No. 13/579,009, filed on Feb. 16, 2011, which claims the benefit of U.S. provisional patent application No. 61/305,082, filed on Feb. 16, 2010, is hereby incorporated by reference.

### FIELD OF THE INVENTION

The present invention is directed to the use of a compound of Formula (I), as herein defined, for the treatment and/or amelioration of diseases, syndromes, disorders, or conditions associated with AR mutant receptors linked to castration-resistant prostate cancer, in a subject in need thereof.

### BACKGROUND OF THE INVENTION

Prostate cancer is the most common non-cutaneous malignancy in men and the second leading cause of death in men from cancer in the western world As a male sexual organ, development of the prostate is highly regulated by androgens, the AR and by the products of androgen dependent genes. During all stages of prostate cancer progression, the disease remains dependent upon androgens. Anti-androgens, including AR antagonists, are used therapeutically to reverse the dependence of the tumor upon the actions of androgen (Scher H, Sawyers C. Biology of progressive, castration-resistant prostate cancer: directed therapies targeting the androgen-receptor signaling axis. J Clin Oncol 2005; 23:8253-8261; Tran C, Ouk S, Clegg N, Chen Y, Watson P, Arora V, et al. Development of a second-generation antiandrogen for treatment of advanced prostate cancer. Science 2009; 324:787-790; Scher H, Fizazi K, Saad F, Taplin M, Sternberg C, Miller K, et al. Increased survival with enzalutamide in prostate cancer after chemotherapy. N Engl J Med 2012; 367:1187-1197). Unfortunately, the efficacy of even second-generation, highly potent AR antagonists, such as MDV-3100 (enzalutamide, Xtandi®), is short-lived in many patients.

AR antagonists have transformed patient care by targeting a key nodal point in tumor cell signaling. However, as with other molecularly targeted cancer therapies across different oncology indications, the emergence of acquired resistance via mutation of the therapeutic target is not uncommon. This is best exemplified by imatinib-treated patients with chronic myeloid leukemia in whom ABL kinase mutations render leukemia cells resistant to imatinib. Multiple next-generation ABL inhibitors have since been developed to circumvent the mutation and with activity in this setting (Gorre M, Mohammed M, Ellwood K, Hsu N, Paquette R, Rao P, Sawyers C. Clinical resistance to STI-571 cancer therapy caused by BCRABL gene mutation or amplification. Science 2001; 293:876-80; O'Hare T, Deininger M W, Eide C A, Clackson T, Druker B J. Targeting the BCR-ABL signaling pathway in therapy-resistant Philadelphia chromosome-positive leukemia. Clin Cancer Res 2011. 17:212-21).

Importantly, the activity of second- and third-generation AR inhibitors indicates that the disease remains “addicted” to a deregulated driver. This has led to the paradigm of sequential therapy targeting the same driver oncogene in distinct resistant states and is applicable herein to targeting of AR and the lineage dependence of AR signaling.

AR mutations that result in receptor promiscuity and the ability of these anti-androgens to exhibit agonist activity might at least partially account for this phenomenon. For example, hydroxyflutamide and bicalutamide act as AR agonists in T877A and W741L/W741C AR mutants, respectively.

In the setting of prostate cancer cells that were rendered castration resistant via overexpression of AR, it has been demonstrated that certain anti-androgen compounds, such as bicalutamide, have a mixed antagonist/agonist profile (Tran C, Ouk S, Clegg N, Chen Y, Watson P, Arora V, et al. Development of a second-generation antiandrogen for treatment of advanced prostate cancer. Science 2009; 324:787-790). This agonist activity helps to explain a clinical observation, called the anti-androgen withdrawal syndrome, whereby about 30% of men who progress on AR antagonists experience a decrease in serum PSA when therapy is discontinued (Scher, H. I. and Kelly, W. K., J Urol 1993 March; 149(3): 607-9). Prostate specific antigen decline after antiandrogen withdrawal: the flutamide withdrawal syndrome.

Accumulating evidence indicates that castration-resistant prostate cancer (CRPC) remains dependent upon AR signaling through reactivation of AR signaling (Yuan X, Balk S. Mechanisms mediating androgen receptor reactivation after castration. Urol Oncol 2009; 27: 36-41; Linja M, Savinainen K, Saramaki O, Tammela T, Vessella R, Visakorpi T. Amplification and overexpression of androgen receptor gene in hormone-refractory prostate cancer. Cancer Res 2001, 61:3550-5; Chen C, Welsbie D, Tran C, Baek S, Chen R, Vessella R, Rosenfeld M, Sawyers C. Molecular determinants of resistance to antiandrogen therapy. Nat Med 2004, 10(1): 33-9). Point mutation in the ligand-binding domain (LBD) of AR accounts for 10-20% of resistance and is characterized by receptor activation, rather than inhibition, by anti-androgen drugs (Beltran H, Yelensky R, Frampton G, Park K, Downing S, MacDonald T, et al. Targeted next-generation sequencing of advanced prostate cancer identifies potential therapeutic targets and disease heterogeneity. Eur Urol 2013; 63(5): 920-6; Bergerat J, Céraline J. Pleiotropic functional properties of androgen receptor mutants in prostate cancer. Hum Mutat 2009; 30(2):145-57). Many of these mutations broaden ligand specificity, and some confer resistance by converting the AR antagonist into an agonist of the mutant receptor (Veldscholte J, Ris-Stalpers C, Kuiper G G, Jenster G, Berrevoets C, Claassen E, van Rooij H C, Trapman J, Brinkmann A O, Mulder E. A mutation in the ligand binding domain of the androgen receptor of human LNCaP cells affects steroid binding characteristics and response to anti-androgens. Biochem Biophys Res Commun. 1990; 173: 534-40; Haapala K, Hyytinen E, Roiha M, Laurila M, Rantala I, Helin H, Koivisto P. Androgen receptor alterations in prostate cancer relapsed during a combined androgen blockade by orchiectomy and bicalutamide. Lab Invest 2001; 81(12):1647-1651; Hara T, Miyazaki J, Araki H, Yamaoka M, Kanzaki N, Kusaka M, Miyamoto M. Novel mutations of androgen receptor: a possible mechanism of bicalutamide withdrawal syndrome. Cancer Res 2003; 63(1):149-153).

One mutation, phenylalanine to leucine at position 876 (F876L) of AR, was recently shown to arise in response to MDV-3100 and ARN-509 in preclinical models and in patients undergoing therapy with ARN-509 (Clegg N, Wongvipat J, Joseph J, Tran C, Ouk S, Dilhas A, et al. ARN-509: a novel antiandrogen for prostate cancer treatment. Cancer Res 2012; 72(6):1494-503; Balbas M, Evans M, Hosfield D, Wongvipat J, Arora V, Watson P, et al. Overcoming mutation-based resistance to antiandrogens with rational drug design. Elife 2013. 2: e00499; Korpal M, Korn J, Gao X, Rakiec D, Ruddy D, Doshi S, et al. An F876L mutation in androgen receptor confers genetic and phenotypic resistance to MDV3100 (enzalutamide). Cancer Discov 2013; 39:1030-1043; Joseph J D, Lu N, Qian J, Sensintaffar J, Shao G, Brigham D, Moon M, Maneval E C, Chen I, Darimont B, Hager J H. A clinically relevant androgen receptor mutation confers resistance to second-generation antiandrogens enzalutamide and ARN-509. Cancer Discov 2013; 3:1020-1029).

AR F876L confers resistance to MDV-3100 and ARN-509. Comprehensive biological studies have demonstrated that prostate cancer cells harboring this mutation continued to grow when treated with either compound. In vitro reporter assays confirmed resistance and demonstrate agonist conversion of both compounds and in tumors engineered to express AR F876L, neither compound controlled tumor growth. Furthermore, the AR F876L mutant is detected in ARN-509-treated patients with progressive CRPC. The mutation was detected in the plasma DNA of patients undergoing longitudinal analysis in 3 of 29 patients eligible for assessment. All 3 of the patients were amongst the 18 patients with an increase in prostate specific antigen (PSA) whilst on drug, indicative of disease progression (Joseph 2013).

Structural modeling of wild-type (WT) and F876L mutated AR bound with MDV-3100, indicated that helices 11 and 12 were differentially displaced. Within the LBD of AR in the F876L mutant, helix 12 is not displaced by MDV-3100 as it is in WT AR, and this allows MDV 3100 to function as an agonist. The compounds described herein are designed to act as antagonists (third-generation), where second-generation compounds are not active.

Therefore, it is an object of the present invention to provide a method of treating and/or ameliorating diseases, syndromes, disorders, or conditions associated with AR mutant receptors linked to castration-resistant prostate cancer, in a subject, including a mammal and/or human, in a subject in need thereof, who has demonstrated resistance to a first or second generation AR antagonist, using a therapeutically effective amount of a pharmaceutical composition comprising a compound of Formula (I).

### SUMMARY OF THE INVENTION

The present invention is directed to a method for treating and/or ameliorating diseases, syndromes, disorders, or conditions associated with AR mutant receptors linked to castration-resistant prostate cancer, in a subject, including a mammal and/or human in need thereof, who has demonstrated resistance to a first or second generation AR antagonist, comprising, consisting of, and/or consisting essentially of, administering to a subject in need thereof, a therapeutically effective amount of a compound of Formula (I)

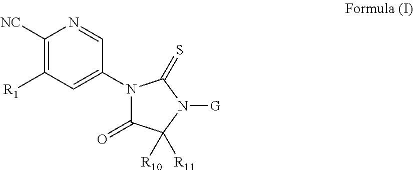

or an enantiomer, diastereomer, or pharmaceutically acceptable salt form thereof; wherein

R₁ is methyl, difluoromethyl, or trifluoromethyl;

G is selected from the group consisting of unsubstituted 1H-indazol-5-yl, unsubstituted isoquinolin-7-yl, unsubstituted pyridin-3-yl, unsubstituted naphthyl, and a phenyl substituent g1;

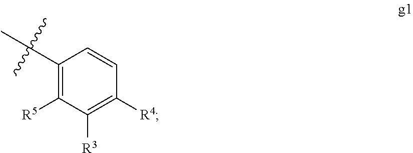

wherein

R³ is selected from hydrogen, fluoro, methyl, trifluoromethoxy, hydroxymethyl, phenyloxy, methoxy, or cyano;

R⁵ is hydrogen, fluoro, or methoxy, such that at least one of R³ and R⁵ is hydrogen;

R⁴ is selected from the group consisting of hydrogen, cyano, fluoro, hydroxy, methoxy, methyl, trifluoromethyl, methylaminosulfonyl, trifluoromethoxy, pyrrolidin-1-ylcarbonyl, piperazin-1-yl, (4-methyl)piperazin-1-yl(C₁₋₃)alkyl, tetrahydropyran-4-yl, and a substituent from i) to v);
i) —C(═O)NH(R_{A}); wherein R_{A} is a substituent selected from hydrogen; C₁₋₆alkyl; 2-hydroxy-2-methyl-propyl; cyclopentylmethyl; 3-hydroxypropyl; cyanomethyl; methoxy(C₂₋₃)alkyl; 3-(cyclopentyl(N-methyl)amino)propyl; ethoxycarbonyl(C₁₋₃)alkyl; 3-(pyrrolidin-1-yl)propyl; morpholin-4-yl(C₂₋₃)alkyl; 4-methylpiperazin-1-yl(C₂₋₃)alkyl; 3-(2-oxopyrrolidin-1-yl)propyl; thienylmethyl; thiazol-2-yl; 2-methylpyrazol-3-yl; furanyl(C₀₋₃)alkyl wherein said furanyl is optionally substituted with a methyl substituent; phenyl(C₀₋₃)alkyl wherein said phenyl is optionally substituted with a chloro or fluoro substituent; pyridinyl(C₀₋₂)alkyl wherein pyridinyl is optionally substituted with a methyl or fluoro substituent; pyrazin-2-ylmethyl; (1-methyl)piperidin-4-yl; and tetrahydropyran-4-yl(C₀₋₁)alkyl;
ii)

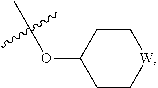

wherein W is selected from NH, N(methyl), N(ethyl), N(2-hydroxyethyl), N(SO₂CH₃), S, O, or SO₂;
iii) —O(C₂₋₃)alkyl-R_{b}; wherein R_{b} is a terminal substituent selected from the group consisting of methoxy, piperazin-1-yl, 4-methylpiperazin-1-yl, piperidin-1-yl, pyridin-2-yl, pyrimidin-2-yl, and pyrrolidin-1-yl;
iv) —OR, wherein R_{c} is phenyl, pyridin-2-yl, pyrimidin-2-yl, pyrimidin-5-yl, or pyrimidin-4-yl;
   and
v) a heteroaryl selected from the group consisting of pyrimidin-5-yl, furanyl, and pyridin-3-yl; wherein said pyridin-3-yl is optionally substituted with a methyl or fluoro substituent; and wherein said furanyl is optionally substituted with a methyl substituent;

R₁₀ and R₁₁ are each a methyl substituent; or R₁₀ and R₁₁ are taken together to form a cyclobutyl or cyclopentyl ring.

The present invention is directed to the use of a compound of Formula (I) as herein defined, for the treatment and/or amelioration of a disease, syndrome, condition, or disorder in a subject, including a mammal and/or human in need thereof, who has demonstrated resistance to a first or second generation AR antagonist, in which the disease, syndrome, condition, or disorder is affected by the antagonism of one or more androgen receptor types, such as prostate cancer, castration-resistant prostate cancer, and metastatic castration-resistant prostate cancer.

The present invention also directed to the use of a pharmaceutical composition comprising, consisting of and/or consisting essentially of a pharmaceutically acceptable carrier, a pharmaceutically acceptable excipient, and/or a pharmaceutically acceptable diluent and a compound of Formula (I), or a pharmaceutically acceptable salt form thereof, for the treatment and/or amelioration of a disease, syndrome, condition, or disorder in a subject, including a mammal and/or human, in need thereof, who has demonstrated resistance to a first or second generation AR antagonist, in which the disease, syndrome, condition, or disorder is affected by the antagonism of one or more androgen receptor types, such as prostate cancer, castration-resistant prostate cancer, and metastatic castration-resistant prostate cancer.

The present invention also is directed to the use of any of the compounds described herein in the preparation of a medicament wherein the medicament is prepared for the treatment and/or amelioration of a disease, syndrome, condition, or disorder in a subject, including a mammal and/or human in need thereof, who has demonstrated resistance to a first or second generation AR antagonist, in which the disease, syndrome, condition, or disorder is affected by the antagonism of one or more androgen receptor types, such as prostate cancer, castration-resistant prostate cancer, and metastatic castration-resistant prostate cancer.

Exemplifying the invention are methods of treating a disease, syndrome, condition, or disorder mediated by one or more androgen receptor types, selected from the group consisting of prostate cancer, castration-resistant prostate cancer, and metastatic castration-resistant prostate cancer, comprising, consisting of, and/or consisting essentially of, administering to a subject in need thereof, who has demonstrated resistance to a first or second generation AR antagonist, a therapeutically effective amount of any of the compounds or pharmaceutical compositions described in the present invention.

In another embodiment, the present invention is directed to a compound of Formula (I) for use in the treatment and/or amelioration of a disease, syndrome, condition, or disorder affected by the antagonism of one or more androgen receptor types, in a patient who has demonstrated resistance to a first or second generation AR antagonist, selected from the group consisting of prostate cancer, castration-resistant prostate cancer, and metastatic castration-resistant prostate cancer.

### DETAILED DESCRIPTION OF THE INVENTION

With reference to substituents, the term “independently” refers to the situation where when more than one substituent is possible, the substituents may be the same or different from each other.

The term “alkyl” whether used alone or as part of a substituent group, refers to straight and branched carbon chains having 1 to 8 carbon atoms. Therefore, designated numbers of carbon atoms (e.g., C₁₋₈) refer independently to the number of carbon atoms in an alkyl moiety or to the alkyl portion of a larger alkyl-containing substituent. In substituent groups with multiple alkyl groups such as, (C₁₋₆alkyl)₂amino-, the C₁₋₆alkyl groups of the dialkylamino may be the same or different.

The term “alkoxy” refers to an —O-alkyl group, wherein the term “alkyl” is as defined above.

The terms “alkenyl” and “alkynyl” refer to straight and branched carbon chains having 2 to 8 carbon atoms, wherein an alkenyl chain contains at least one double bond and an alkynyl chain contains at least one triple bond.

The term “cycloalkyl” refers to saturated or partially saturated, monocyclic or polycyclic hydrocarbon rings of 3 to 14 carbon atoms. Examples of such rings include cyclopropyl, cyclobutyl, cyclopentyl, cyclohexyl, cycloheptyl, and adamantyl.

The term “heterocyclyl” refers to a nonaromatic monocyclic or bicyclic ring system having 3 to 10 ring members that include at least 1 carbon atom and from 1 to 4 heteroatoms independently selected from N, O, and S. Included within the term heterocyclyl is a nonaromatic cyclic ring of 5 to 7 members in which 1 to 2 members are N, or a nonaromatic cyclic ring of 5 to 7 members in which 0, 1 or 2 members are N and up to 2 members are 0 or S and at least one member must be either N, O, or S; wherein, optionally, the ring contains 0 to 1 unsaturated bonds, and, optionally, when the ring is of 6 or 7 members, it contains up to 2 unsaturated bonds. The carbon atom ring members that form a heterocycle ring may be fully saturated or partially saturated. The term “heterocyclyl” also includes two 5 membered monocyclic heterocycloalkyl groups bridged to form a bicyclic ring. Such groups are not considered to be fully aromatic and are not referred to as heteroaryl groups. When a heterocycle is bicyclic, both rings of the heterocycle are non-aromatic and at least one of the rings contains a heteroatom ring member. Examples of heterocycle groups include, and are not limited to, pyrrolinyl (including 2H-pyrrole, 2-pyrrolinyl or 3-pyrrolinyl), pyrrolidinyl, imidazolinyl, imidazolidinyl, pyrazolinyl, pyrazolidinyl, piperidinyl, morpholinyl, thiomorpholinyl, and piperazinyl. Unless otherwise noted, the heterocycle is attached to its pendant group at any heteroatom or carbon atom that results in a stable structure.

The term “aryl” refers to an unsaturated, aromatic monocyclic or bicyclic ring of 6 to 10 carbon members. Examples of aryl rings include phenyl and naphthalenyl. The term “heteroaryl” refers to an aromatic monocyclic or bicyclic aromatic ring system having 5 to 10 ring members and which contains carbon atoms and from 1 to 4 heteroatoms independently selected from the group consisting of N, O, and S. Included within the term heteroaryl are aromatic rings of 5 or 6 members wherein the ring consists of carbon atoms and has at least one heteroatom member. Suitable heteroatoms include nitrogen, oxygen, and sulfur. In the case of 5 membered rings, the heteroaryl ring preferably contains one member of nitrogen, oxygen or sulfur and, in addition, up to 3 additional nitrogens. In the case of 6 membered rings, the heteroaryl ring preferably contains from 1 to 3 nitrogen atoms. For the case wherein the 6 membered ring has 3 nitrogens, at most 2 nitrogen atoms are adjacent. Examples of heteroaryl groups include furyl, thienyl, pyrrolyl, oxazolyl, thiazolyl, imidazolyl, pyrazolyl, isoxazolyl, isothiazolyl, oxadiazolyl, triazolyl, thiadiazolyl, pyridinyl, pyridazinyl, pyrimidinyl, pyrazinyl, indolyl, isoindolyl, benzofuryl, benzothienyl, indazolyl, benzimidazolyl, benzothiazolyl, benzoxazolyl, benzisoxazolyl, benzothiadiazolyl, benzotriazolyl, quinolinyl, isoquinolinyl and quinazolinyl. Unless otherwise noted, the heteroaryl is attached to its pendant group at any heteroatom or carbon atom that results in a stable structure.

The term “halogen” or “halo” refers to fluorine, chlorine, bromine and iodine atoms.

The term “carboxy” refers to the group —C(═O)OH.

The term “formyl” refers to the group —C(═O)H.

The term “oxo” or “oxido” refers to the group (═O).

Whenever the term “alkyl” or “aryl” or either of their prefix roots appear in a name of a substituent (e.g., arylalkyl, alkylamino) the name is to be interpreted as including those limitations given above for “alkyl” and “aryl.” Designated numbers of carbon atoms (e.g., C₁-C₆) refer independently to the number of carbon atoms in an alkyl moiety, an aryl moiety, or in the alkyl portion of a larger substituent in which alkyl appears as its prefix root. For alkyl and alkoxy substituents, the designated number of carbon atoms includes all of the independent members included within a given range specified. For example C₁₋₆ alkyl would include methyl, ethyl, propyl, butyl, pentyl and hexyl individually as well as sub-combinations thereof (e.g., C₁₋₂, C₁₋₃, C₁₋₄, C₁₋₅, C₂₋₆, C₃₋₆, C₄₋₆, C₅₋₆, C₂₋₅, etc.).

In general, under standard nomenclature rules used throughout this disclosure, the terminal portion of the designated side chain is described first followed by the adjacent functionality toward the point of attachment. Thus, for example, a “C₁-C₆ alkylcarbonyl” substituent refers to a group of the formula:

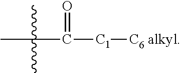

The label “R” at a stereocenter designates that the stereocenter is purely of the R-configuration as defined in the art; likewise, the label “S” means that the stereocenter is purely of the S-configuration. As used herein, the labels “*R” or “*S” at a stereocenter are used to designate that the stereocenter is of pure but unknown absolute configuration. As used herein, the label “RS” refers to a stereocenter that exists as a mixture of the R- and S-configurations.

A compound containing one stereocenter drawn without a stereo bond designation is a mixture of two enantiomers. A compound containing two stereocenters both drawn without stereo bond designations is a mixture of four diastereomers. A compound with two stereocenters both labeled “RS” and drawn with stereo bond designations is a mixture of two enantiomers with relative stereochemistry as drawn. A compound with two stereocenters both labeled “*RS” and drawn with stereo bond designations is a mixture of two enantiomers with a single, but unknown, relative stereochemistry.

Unlabeled stereocenters drawn without stereo bond designations are mixtures of the R- and S-configurations. For unlabeled stereocenters drawn with stereo bond designations, the relative and absolute stereochemistry is as depicted.

Unless otherwise noted, it is intended that the definition of any substituent or variable at a particular location in a molecule be independent of its definitions elsewhere in that molecule. It is understood that substituents and substitution patterns on the compounds of the present invention can be selected by one of ordinary skill in the art to provide compounds that are chemically stable and that can be readily synthesized by techniques known in the art as well as those methods set forth herein.

The term “subject” refers to an animal, preferably a mammal, most preferably a human, who has been the object of treatment, observation or experiment.

The term “therapeutically effective amount” refers to an amount of an active compound or pharmaceutical agent, including a compound of the present invention, which elicits the biological or medicinal response in a tissue system, animal or human that is being sought by a researcher, veterinarian, medical doctor or other clinician, which includes alleviation or partial alleviation of the symptoms of the disease, syndrome, condition, or disorder being treated.

The term “composition” refers to a pharmaceutical product that includes the specified ingredients sometimes in therapeutically effective amounts, as well as any product that results, directly, or indirectly, from combinations of the specified ingredients in the specified amounts.

The term “androgen receptor” as used herein is intended to include the wild-type androgen receptor as well as AR mutant receptors associated with castration-resistant prostate cancer.

The term “AR-mediated” refers to any disease, syndrome, condition, or disorder that might occur in the absence of androgen receptors but can occur in the presence of androgen receptors. Suitable examples of include, but are not limited to, prostate cancer, castration-resistant prostate cancer, and metastatic castration-resistant prostate cancer.

The term “androgen-dependent disorder” refers to any disorder that can benefit from a decrease in androgen stimulation and includes pathological conditions that depend on androgen stimulation. An “androgen-dependent disorder” can result from an excessive accumulation of testosterone or other androgenic hormone, increased sensitivity of androgen receptors to androgen, or an increase in androgen-stimulated transcription.

Examples of “androgen-dependent disorders” include prostate cancer and disorders such as, for example, acne, seborrhea, hirsutism, alopecia, and hidradenitis suppurativa.

As used herein, the term “anti-androgen” refers to a group of hormone receptor antagonist compounds that are capable of preventing or inhibiting the biologic effects of androgens on normally responsive tissues in the body. In some embodiments, an anti-androgen is a small molecule. In some embodiments, an anti-androgen is an AR antagonist. In some embodiments, an anti-androgen is an AR full antagonist. In some embodiments, an anti-androgen is a first-generation anti-androgen. In some embodiments, an anti-androgen is a second-generation anti-androgen. In some embodiments, an anti-androgen is a third-generation anti-androgen.

As used herein, the term “AR antagonist” or “AR inhibitor” are used interchangeably and refer to an agent that inhibits or reduces at least one activity of an AR polypeptide. Exemplary AR activities include, but are not limited to, co-activator binding, DNA binding, ligand binding, or nuclear translocation.

As used herein, a “full antagonist” refers to an antagonist which, at an effective concentration, essentially completely inhibits an activity of an AR polypeptide. As used herein, a “partial antagonist” refers an antagonist that is capable of partially inhibiting an activity of an AR polypeptide, but that, even at a highest concentration is not a full antagonist. By ‘essentially completely’ is meant at least about 80%, at least about 90%, at least about 95%, at least about 96%, at least about 97%, at least about 98% at least about 99%, or greater inhibition of the activity of an AR polypeptide.

As used herein, the term “first-generation anti-androgen” refers to an agent that exhibits antagonist activity against a wild-type AR polypeptide. However, first-generation anti-androgens differ from second-generation anti-androgens in that first-generation anti-androgens can potentially act as agonists in castration resistant prostate cancers (CRPC). Exemplary first-generation anti-androgens include, but are not limited to, flutamide, nilutamide and bicalutamide.

As used herein, the term “second-generation anti-androgen” refers to an agent that exhibits full antagonist activity against a wild-type AR polypeptide. Second-generation anti-androgens differ from first-generation anti-androgens in that second-generation anti-androgens act as full antagonists in cells expressing elevated levels of AR, such as for example, in castration resistant prostate cancers (CRPC). Exemplary second-generation anti-androgens include 4-[7-(6-cyano-5-trifluoromethylpyridin-3-yl)-8-oxo-6-thioxo-5,7-diazaspiro[3.4]oct-5-yl]-2-fluoro-N methylbenzamide (also known as ARN-509; CAS No. 956104-40-8); 4-(3-(4-cyano-3-(trifluoromethyl)phenyl)-5,5-dimethyl-4-oxo-2-thioxoimidazolidin-1-yl)-2-fluoro-N-methylbenzamide (also known as MDV3100 or enzalutamide; CAS No: 915087-33-1) and RD162 (CAS No. 915087-27-3). In some embodiments, a second-generation anti-androgen binds to an AR polypeptide at or near the ligand binding site of the AR polypeptide.

As used herein, the term “third-generation anti-androgen” refers to an agent that exhibits full antagonist activity against a wild-type AR polypeptide and against mutant forms of the AR polypeptide, with mutations arising in the ligand binding domain (LBD) of the AR polypeptide as set forth below. Third-generation anti-androgens retain the differentiation from first-generation anti-androgens in that third-generation anti-androgens act as full antagonists in cells expressing elevated levels of AR, such as for example, in castration resistant prostate cancers (CRPC).

As used herein, the term “mutant” refers to an altered (as compared with a reference) nucleic acid or polypeptide, or to a cell or organism containing or expressing such altered nucleic acid or polypeptide.

As used herein, unless otherwise noted, the term “affect” or “affected” (when referring to a disease, syndrome, condition or disorder that is affected by antagonism of AR) includes a reduction in the frequency and/or severity of one or more symptoms or manifestations of said disease, syndrome, condition or disorder; and/or include the prevention of the development of one or more symptoms or manifestations of said disease, syndrome, condition or disorder or the development of the disease, condition, syndrome or disorder.

The compounds of the instant invention are useful in methods for treating or ameliorating a disease, a syndrome, a condition or a disorder that is affected by the antagonism of one or more AR receptors. Such methods comprise, consist of and/or consist essentially of administering to a subject, including an animal, a mammal, and a human in need of such treatment, amelioration and/or prevention, who has demonstrated resistance to a first or second generation AR antagonist, a therapeutically effective amount of a compound of Formula (I), or an enantiomer, diastereomer, solvate or pharmaceutically acceptable salt thereof.

One embodiment of the present invention is directed to a method of treating an androgen receptor dependent or androgen receptor mediated disease or condition in a subject in need thereof, including an animal, a mammal, and a human in need of such treatment, who has demonstrated resistance to a first or second generation AR antagonist, comprising administering to the subject a therapeutically effective amount of a compound of Formula (I).

In another embodiment, the androgen receptor dependent or androgen receptor mediated disease or condition is selected from benign prostate hyperplasia, hirsutism, acne, adenomas and neoplasies of the prostate, benign or malignant tumor cells containing the androgen receptor, hyperpilosity, seborrhea, endometriosis, polycystic ovary syndrome, androgenic alopecia, hypogonadism, osteroporosis, suppression of spermatogenesis, libido, cachexia, anorexia, androgen supplementation for age related decreased testosterone levels, prostate cancer, breast cancer, endometrial cancer, uterine cancer, hot flashes, and Kennedy's disease muscle atrophy and weakness, skin atrophy, bone loss, anemia, arteriosclerosis, cardiovasculasr disease, loss of energy, loss of well-being, type 2 diabetes, or abdominal fat accumulation.

In particular, the compounds of Formula (I), or an enantiomer, diastereomer, solvate or pharmaceutically acceptable salt form thereof are useful for treating or ameliorating diseases, syndromes, conditions, or disorders such as prostate cancer, castration-resistant prostate cancer, and metastatic castration-resistant prostate cancer.

More particularly, the compounds of Formula (I), or an enantiomer, diastereomer, solvate or pharmaceutically acceptable salt form thereof, are useful for treating or ameliorating prostate cancer, castration-resistant prostate cancer, and metastatic castration-resistant prostate cancer, comprising administering to a subject in need thereof, who has demonstrated resistance to a first or second generation AR antagonist, a therapeutically effective amount of a compound of Formula (I), or an enantiomer, diastereomer, solvate or pharmaceutically acceptable salt form thereof as herein defined.

In an embodiment, the present invention is directed to a method for treating and/or ameliorating diseases, syndromes, disorders, or conditions associated with AR mutant receptors linked to castration-resistant prostate cancer, in a subject, including a mammal and/or human, in need thereof, who has demonstrated resistance to a first or second generation AR antagonist, comprising, consisting of, and/or consisting essentially of, administering to a subject in need thereof, a therapeutically effective amount of a compound of Formula (I)

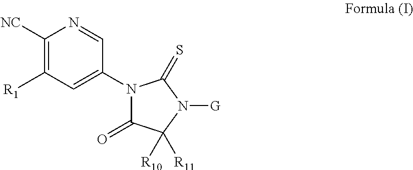

or an enantiomer, diastereomer, or pharmaceutically acceptable salt form thereof;
wherein,
AA) R₁ is methyl or trifluoromethyl;
BB) G is selected from the group consisting of unsubstituted isoquinolin-7-yl, unsubstituted pyridin-3-yl, unsubstituted naphthyl, and a phenyl substituent g1;

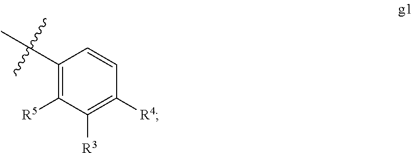

wherein
R³ is selected from hydrogen, fluoro, methyl, phenyloxy, or methoxy;
R⁵ is hydrogen;
R⁴ is selected from the group consisting of hydrogen, hydroxy, methoxy, methyl, methylaminosulfonyl, trifluoromethoxy, pyrrolidin-1-ylcarbonyl, piperazin-1-yl, (4-methyl)piperazin-1-yl(C₁₋₃)alkyl, and a substituent from i) to v);
i) —C(═O)NH(R_{A}); wherein R_{A} is a substituent selected from the group consisting of C₁₋₆alkyl; 2-hydroxy-2-methyl-propyl; cyclopentylmethyl; 3-hydroxypropyl; methoxy(C₂₋₃)alkyl; 3-(cyclopentyl(N-methyl)amino)propyl; ethoxycarbonyl(C₁₋₃)alkyl; morpholin-4-yl(C₂₋₃)alkyl; 3-(2-oxopyrrolidin-1-yl)propyl; thienylmethyl; thiazol-2-yl; 2-methylpyrazol-3-yl; furanyl(C₀₋₃)alkyl wherein said furanyl is optionally substituted with a methyl substituent; phenyl(C₀₋₃)alkyl wherein said phenyl is optionally substituted with a chloro or fluoro substituent; unsubstituted pyridinyl(C₀₋₂)alkyl; pyrazin-2-ylmethyl; and tetrahydropyran-4-yl(C₀₋₁)alkyl;
ii)

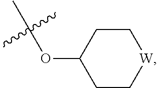

wherein W is selected from NH, N(methyl), N(ethyl), N(2-hydroxyethyl), S, or SO₂;
iii) —O(C₂₋₃)alkyl-R_{b}; wherein R_{b} is a terminal substituent selected from the group consisting of 4-methylpiperazin-1-yl, pyrimidin-2-yl, pyridin-2-yl, and pyrrolidin-1-yl;
iv) —OR, wherein R, is pyrimidin-4-yl;

and
v) a heteroaryl selected from the group consisting of furanyl and pyridin-3-yl; wherein said furanyl is optionally substituted with a methyl substituent;
CC) G is selected from the group consisting of unsubstituted isoquinolin-7-yl, unsubstituted pyridin-3-yl, unsubstituted naphthyl, and a phenyl substituent g1;

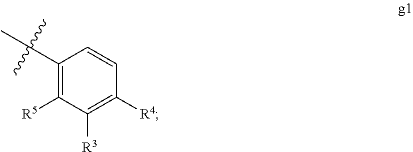

wherein
R³ is selected from fluoro, methyl, or phenyloxy;
R⁵ is hydrogen;
R⁴ is selected from the group consisting of methyl, methylaminosulfonyl, trifluoromethoxy, piperazin-1-yl, (4-methyl)piperazin-1-yl(C₁₋₃)alkyl, and a substituent from i) to iv);
i) —C(═O)NH(R_{A}); wherein R_{A} is a substituent selected from the group consisting of C₁₋₆alkyl; 2-hydroxy-2-methyl-propyl; cyclopentylmethyl; 3-hydroxypropyl; methoxy(C₂₋₃)alkyl; ethoxycarbonyl(C₁₋₃)alkyl; morpholin-4-yl(C₂₋₃)alkyl; 3-(2-oxopyrrolidin-1-yl)propyl; thienylmethyl; 2-methylpyrazol-3-yl; furanyl(C₀₋₃)alkyl wherein said furanyl is optionally substituted with a methyl substituent; phenyl(C₀₋₃)alkyl wherein said phenyl is optionally substituted with fluoro substituent; unsubstituted pyridinyl(C₀₋₂)alkyl; and tetrahydropyran-4-yl(C₀₋₁)alkyl;
ii)

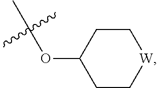

wherein W is selected from NH, N(methyl), S, or SO₂;
iii) —O(C₂₋₃)alkyl-R_{b}; wherein R_{b} is a terminal substituent selected from the group consisting of 4-methylpiperazin-1-yl and pyridin-2-yl;

and
iv) pyridin-3-yl;
DD) G is selected from the group consisting of unsubstituted isoquinolin-7-yl, unsubstituted pyridin-3-yl or a phenyl substituent g1;

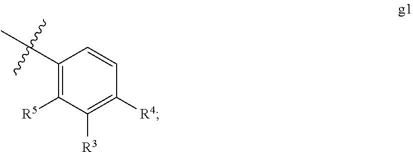

wherein
R³ is selected from hydrogen, fluoro or methyl;
R⁵ is hydrogen;
R⁴ is selected from the group consisting of piperazin-1-yl and a substituent from i) to iv);
i) —C(═O)NH(R_{A}); wherein R_{A} is a substituent selected from the group consisting of unsubstituted pyridinyl(C₀₋₂)alkyl and tetrahydropyran-4-yl(C₀₋₁)alkyl;
ii)

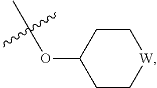

wherein W is selected from N(methyl), S, or SO₂;
iii) —O(C₂₋₃)alkyl-R_{b}; wherein R_{b} is 4-methylpiperazin-1-yl;
   and
iv) a heteroaryl that is pyridin-3-yl;
EE) R⁴ is selected from the group consisting of 2-(pyridin-2-yl)ethylaminocarbonyl, 2-(pyridin-4-yl)ethylaminocarbonyl, tetrahydrothiopyran-4-yloxy, methylaminocarbonyl, (2-fluorophenyl)aminocarbonyl, 2-(4-methylpiperazin-1-yl)ethoxy, piperizin-1-yl, (1,1-dioxothian-4-yl)oxy, (1-methyl-piperidin-4-yl)oxy, tetrahydropyran-4-ylmethylaminocarbonyl, and tetrahydropyran-4-ylaminocarbonyl;
FF) R₁₀ and R₁₁ are each a methyl substituent; or R₁₀ and R₁₁ are taken together to form a cyclobutyl ring;

and any combination of embodiments AA) through FF) above, provided that it is understood that combinations in which different embodiments of the same substituent would be combined are excluded.

In an embodiment, the present invention is directed to a method for treating and/or ameliorating diseases, syndromes, disorders, or conditions associated with AR mutant receptors linked to castration-resistant prostate cancer, in a subject, including a mammal and/or human, in need thereof, who has demonstrated resistance to a first or second generation AR antagonist, comprising, consisting of, and/or consisting essentially of, administering to a subject in need thereof, a therapeutically effective amount of a compound of Formula (I)

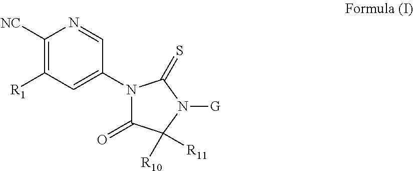

or an enantiomer, diastereomer, or pharmaceutically acceptable salt form thereof;
wherein,

R₁ is methyl, difluoromethyl, or trifluoromethyl;

G is selected from the group consisting of unsubstituted isoquinolin-7-yl, unsubstituted pyridin-3-yl, unsubstituted naphthyl, and a phenyl substituent g1;

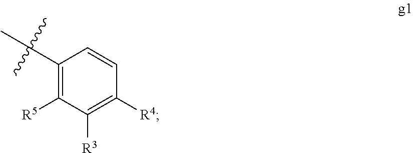

wherein

R³ is selected from hydrogen, fluoro, methyl, phenyloxy, or methoxy;

R⁵ is hydrogen;

R⁴ is selected from the group consisting of hydrogen, hydroxy, methoxy, methyl, methylaminosulfonyl, trifluoromethoxy, pyrrolidin-1-ylcarbonyl, piperazin-1-yl, (4-methyl)piperazin-1-yl(C₁₋₃)alkyl, and a substituent from i) to v);

i) —C(═O)NH(R_{A}); wherein R_{A} is a substituent selected from the group consisting of C₁₋₆alkyl; 2-hydroxy-2-methyl-propyl; cyclopentylmethyl; 3-hydroxypropyl; methoxy(C₂₋₃)alkyl; 3-(cyclopentyl(N-methyl)amino)propyl; ethoxycarbonyl(C₁₋₃)alkyl; morpholin-4-yl(C₂₋₃)alkyl; 3-(2-oxopyrrolidin-1-yl)propyl; thienylmethyl; thiazol-2-yl; 2-methylpyrazol-3-yl; furanyl(C₀₋₃)alkyl wherein said furanyl is optionally substituted with a methyl substituent; phenyl(C₀₋₃)alkyl wherein said phenyl is optionally substituted with a chloro or fluoro substituent; unsubstituted pyridinyl(C₀₋₂)alkyl; pyrazin-2-ylmethyl; and tetrahydropyran-4-yl(C₀₋₁)alkyl;

ii)

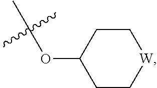

wherein W is selected from NH, N(methyl), N(ethyl), N(2-hydroxyethyl), S, or SO₂;

iii) —O(C₂₋₃)alkyl-R_{b}; wherein R_{b} is a terminal substituent selected from the group consisting of 4-methylpiperazin-1-yl, pyrimidin-2-yl, pyridin-2-yl, and pyrrolidin-1-yl;

iv) —OR, wherein R_{c} is pyrimidin-4-yl;

and

v) a heteroaryl selected from the group consisting of furanyl and pyridin-3-yl; wherein said furanyl is optionally substituted with a methyl substituent;

R₁₀ and R₁₁ are each a methyl substituent; or R₁₀ and R₁₁ are taken together to form a cyclobutyl or cyclopentyl ring.

In an embodiment, the present invention is directed to a method for treating and/or ameliorating diseases, syndromes, disorders, or conditions associated with AR mutant receptors linked to castration-resistant prostate cancer, in a subject, including a mammal and/or human, in need thereof, who has demonstrated resistance to a first or second generation AR antagonist, comprising, consisting of, and/or consisting essentially of, administering to a subject in need thereof, a therapeutically effective amount of a compound of Formula (I)

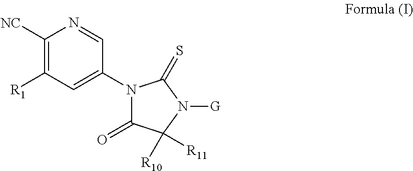

or an enantiomer, diastereomer, or pharmaceutically acceptable salt form thereof;
wherein,

R₁ is methyl, difluoromethyl, or trifluoromethyl;

G is selected from the group consisting of unsubstituted isoquinolin-7-yl, unsubstituted pyridin-3-yl, unsubstituted naphthyl, and a phenyl substituent g1;

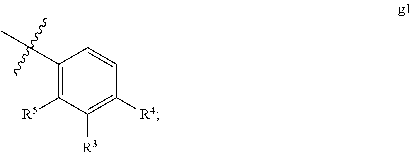

wherein

R³ is selected from fluoro, methyl, or phenyloxy;

R⁵ is hydrogen;

R⁴ is selected from the group consisting of methyl, methylaminosulfonyl, trifluoromethoxy, piperazin-1-yl, (4-methyl)piperazin-1-yl(C₁₋₃)alkyl, and a substituent from i) to iv);

i) —C(═O)NH(R_{A}); wherein R_{A} is a substituent selected from the group consisting of C₁₋₆alkyl; 2-hydroxy-2-methyl-propyl; cyclopentylmethyl; 3-hydroxypropyl; methoxy(C₂₋₃)alkyl; ethoxycarbonyl(C₁₋₃)alkyl; morpholin-4-yl(C₂₋₃)alkyl; 3-(2-oxopyrrolidin-1-yl)propyl; thienylmethyl; 2-methylpyrazol-3-yl; furanyl(C₀₋₃)alkyl wherein said furanyl is optionally substituted with a methyl substituent; phenyl(C₀₋₃)alkyl wherein said phenyl is optionally substituted with a fluoro substituent; unsubstituted pyridinyl(C₀₋₂)alkyl; and tetrahydropyran-4-yl(C₀₋₁)alkyl;

ii)

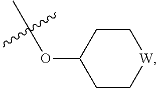

wherein W is selected from NH, N(methyl), S, or SO₂;

iii) —O(C₂₋₃)alkyl-R_{b}; wherein R_{b} is a terminal substituent selected from the group consisting of 4-methylpiperazin-1-yl, and pyridin-2-yl;

and

iv) pyridin-3-yl;

R₁₀ and R₁₁ are each a methyl substituent; or R₁₀ and R₁₁ are taken together to form a cyclobutyl or cyclopentyl ring.

In an embodiment, the present invention is directed to a method for treating and/or ameliorating diseases, syndromes, disorders, or conditions associated with AR mutant receptors linked to castration-resistant prostate cancer, in a subject, including a mammal and/or human, in need thereof, who has demonstrated resistance to a first or second generation AR antagonist, comprising, consisting of, and/or consisting essentially of, administering to a subject in need thereof, a therapeutically effective amount of a compound of Formula (I)

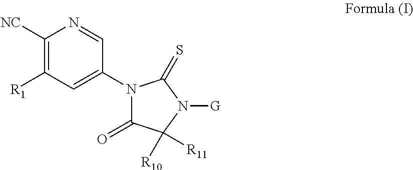

or an enantiomer, diastereomer, or pharmaceutically acceptable salt form thereof;
wherein,

R₁ is methyl or trifluoromethyl;

G is selected from the group consisting of unsubstituted isoquinolin-7-yl, unsubstituted pyridin-3-yl, and a substituent g1;

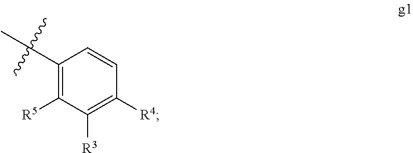

wherein

R³ is selected from hydrogen, fluoro, or methyl;

R⁵ is hydrogen;

R⁴ is selected from the group consisting of piperazin-1-yl and a substituent from i) to iv);

i) —C(═O)NH(R_{A}); wherein R_{A} is a substituent selected from the group consisting of unsubstituted pyridinyl(C₀₋₂)alkyl and tetrahydropyran-4-yl(C₀₋₁)alkyl;

ii)

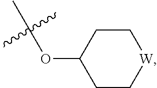

wherein W is selected from N(methyl), S, or SO₂;

iii) —O(C₂₋₃)alkyl-R_{b}; wherein R_{b} is 4-methylpiperazin-1-yl;

and

iv) pyridin-3-yl;

R₁₀ and R₁₁ are each a methyl substituent; or R₁₀ and R₁₁ are taken together to form a cyclobutyl ring.

In an embodiment, the present invention is directed to a method for treating and/or ameliorating diseases, syndromes, disorders, or conditions associated with AR mutant receptors linked to castration-resistant prostate cancer, in a subject, including a mammal and/or human, in need thereof, who has demonstrated resistance to a first or second generation AR antagonist, comprising, consisting of, and/or consisting essentially of, administering to a subject in need thereof, a therapeutically effective amount of a compound of Formula (I)

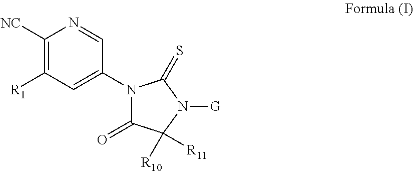

or an enantiomer, diastereomer, or pharmaceutically acceptable salt form thereof;
wherein,

R₁ is methyl or trifluoromethyl;

G is selected from the group consisting of unsubstituted pyridin-3-yl, unsubstituted isoquinolin-7-yl, and a substituent g1

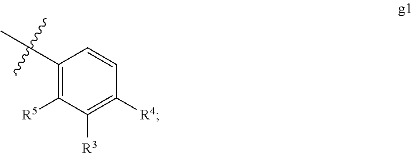

wherein

R³ is selected from hydrogen, fluoro or methyl;

R⁵ is hydrogen;

R⁴ is selected from the group consisting of 2-(pyridin-2-yl)ethylaminocarbonyl, 2-(pyridin-4-yl)ethylaminocarbonyl, tetrahydrothiopyran-4-yloxy, methylaminocarbonyl, (2-fluorophenyl)aminocarbonyl, 2-(4-methylpiperazin-1-yl)ethoxy, piperizin-1-yl, (1,1-dioxothian-4-yl)oxy, (1-methyl-piperidin-4-yl)oxy, tetrahydropyran-4-ylmethylaminocarbonyl, and tetrahydropyran-4-ylaminocarbonyl;

R₁₀ and R₁₁ are each a methyl substituent; or R₁₀ and R₁₁ are taken together to form a cyclobutyl ring.

A further embodiment of the present invention is directed to a method for treating and/or ameliorating diseases, syndromes, disorders, or conditions associated with AR mutant receptors linked to castration-resistant prostate cancer, in a subject, including a mammal and/or human, in need thereof, who has demonstrated resistance to a first or second generation AR antagonist, comprising, consisting of, and/or consisting essentially of, administering to a subject in need thereof, a therapeutically effective amount of a compound of Formula (I), as exemplified in the listing in Table 1, below.

TABLE 1

| | Cpd. | |
|---|---|---|
| Structure | No. | Cpd Name |
| 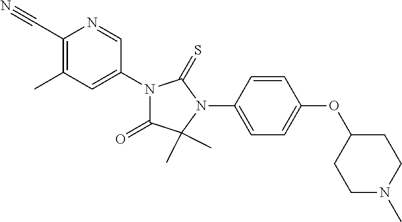 [SMILES: Cc1cc(N2C(=O)C(C)(C)N(c3ccc(OC4CCN(C)CC4)cc3)C2=S)cnc1C#N] | 1 | 5-[4,4-dimethyl-3-[4-[(1-methyl-4- piperidyl)oxy]phenyl]-5-oxo-2-thioxo- imidazolidin-1-yl]-3-methyl-pyridine- 2-carbonitrile |
| 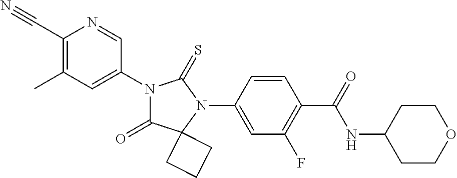 [SMILES: Cc1cc(N2C(=O)C3(CCC3)N(c3ccc(C(=O)NC4CCOCC4)c(F)c3)C2=S)cnc1C#N] | 2 | 4-[6-(6-cyano-5-methyl-3-pyridyl)-5- oxo-7-thioxo-6,8-diazaspiro[3.4]octan- 8-yl]-2-fluoro-N-tetrahydropyran-4-yl- benzamide |
| 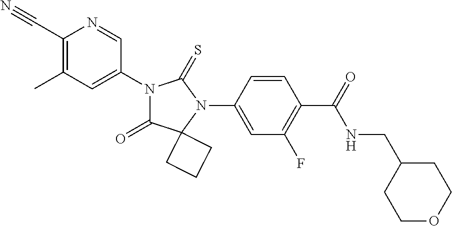 [SMILES: Cc1cc(N2C(=O)C3(CCC3)N(c3ccc(C(=O)NCC4CCOCC4)c(F)c3)C2=S)cnc1C#N] | 3 | 4-[6-(6-cyano-5-methyl-3-pyridyl)-5- oxo-7-thioxo-6,8-diazaspiro[3.4]octan- 8-yl]-2-fluoro-N-(tetrahydropyran-4- ylmethyl)benzamide |
| 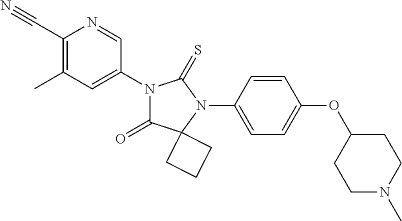 [SMILES: Cc1cc(N2C(=O)C3(CCC3)N(c3ccc(OC4CCN(C)CC4)cc3)C2=S)cnc1C#N] | 4 | 3-methyl-5-[8-[4-[(1-methyl-4- piperidyl)oxy]phenyl]-5-oxo-7-thioxo- 6,8-diazaspiro[3.4]octan-6-yl]pyridine- 2-carbonitrile |
| 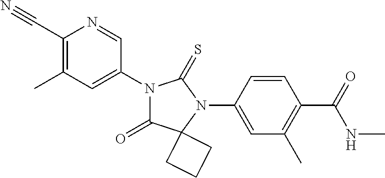 [SMILES: CNC(=O)c1ccc(N2C(=S)N(c3cnc(C#N)c(C)c3)C(=O)C23CCC3)cc1C] | 5 | 4-[6-(6-cyano-5-methyl-3-pyridyl)-5- oxo-7-thioxo-6,8-diazaspiro[3.4]octan- 8-yl]-N,2-dimethyl-benzamide |
| 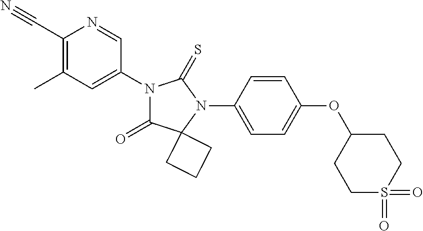 [SMILES: Cc1cc(N2C(=O)C3(CCC3)N(c3ccc(OC4CCS(=O)(=O)CC4)cc3)C2=S)cnc1C#N] | 6 | 5-[8-[4-(1,1-dioxothian-4- yl)oxyphenyl]-5-oxo-7-thioxo-6,8- diazaspiro[3.4]octan-6-yl]-3-methyl- pyridine-2-carbonitrile |
| 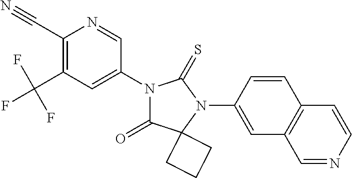 [SMILES: N#Cc1ncc(N2C(=O)C3(CCC3)N(c3ccc4ccncc4c3)C2=S)cc1C(F)(F)F] | 7 | 5-[8-(7-isoquinolyl)-5-oxo-7-thioxo- 6,8-diazaspiro[3.4]octan-6-yl]-3- (trifluoromethyl)pyridine-2-carbonitrile |
| 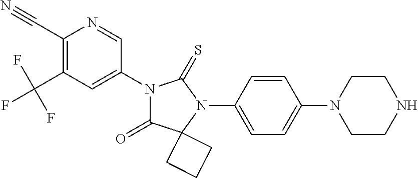 [SMILES: N#Cc1ncc(N2C(=O)C3(CCC3)N(c3ccc(N4CCNCC4)cc3)C2=S)cc1C(F)(F)F] | 8 | 5-[5-oxo-8-(4-piperazin-1-ylphenyl)-7- thioxo-6,8-diazaspiro[3.4]octan-6-yl]- 3-(trifluoromethyl)pyridine-2- carbonitrile |
| 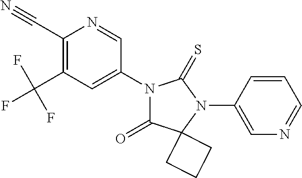 [SMILES: N#Cc1ncc(N2C(=O)C3(CCC3)N(c3cccnc3)C2=S)cc1C(F)(F)F] | 9 | 5-[5-oxo-8-(3-pyridyl)-7-thioxo-6,8- diazaspiro[3.4]octan-6-yl]-3- (trifluoromethyl)pyridine-2-carbonitrile |
| 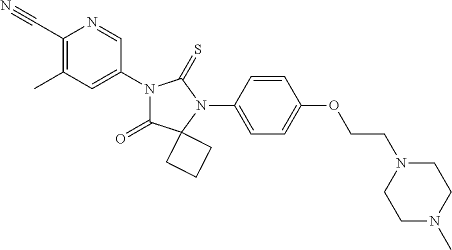 [SMILES: Cc1cc(N2C(=O)C3(CCC3)N(c3ccc(OCCN4CCN(C)CC4)cc3)C2=S)cnc1C#N] | 10 | 3-methyl-5-[8-[4-[2-(4- methylpiperazin-1-yl)ethoxy]phenyl]-5- oxo-7-thioxo-6,8-diazaspiro[3.4]octan- 6-yl]pyridine-2-carbonitrile |
| 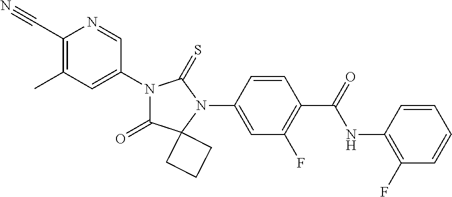 [SMILES: Cc1cc(N2C(=O)C3(CCC3)N(c3ccc(C(=O)Nc4ccccc4F)c(F)c3)C2=S)cnc1C#N] | 11 | 4-[6-(6-cyano-5-methyl-3-pyridyl)-5- oxo-7-thioxo-6,8-diazaspiro[3.4]octan- 8-yl]-2-fluoro-N-(2- fluorophenyl)benzamide |
| 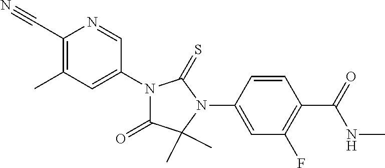 [SMILES: CNC(=O)c1ccc(N2C(=S)N(c3cnc(C#N)c(C)c3)C(=O)C2(C)C)cc1F] | 12 | 4-[3-(6-cyano-5-methyl-3-pyridyl)-5,5- dimethyl-4-oxo-2-thioxo-imidazolidin- 1-yl]-2-fluoro-N-methyl-benzamide |
| 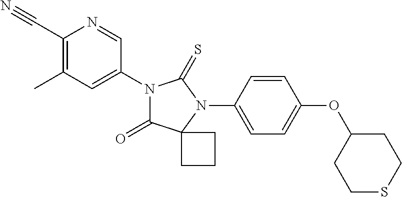 [SMILES: Cc1cc(N2C(=O)C3(CCC3)N(c3ccc(OC4CCSCC4)cc3)C2=S)cnc1C#N] | 13 | 3-methyl-5-[5-oxo-8-(4- tetrahydrothiopyran-4-yloxyphenyl)-7- thioxo-6,8-diazaspiro[3.4]octan-6- yl]pyridine-2-carbonitrile |
| 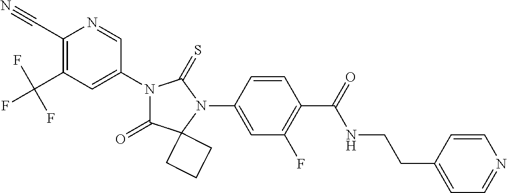 [SMILES: N#Cc1ncc(N2C(=O)C3(CCC3)N(c3ccc(C(=O)NCCc4ccncc4)c(F)c3)C2=S)cc1C(F)(F)F] | 14 | 4-[6-[6-cyano-5-(trifluoromethyl)-3- pyridyl]-5-oxo-7-thioxo-6,8- diazaspiro[3.4]octan-8-yl]-2-fluoro-N- [2-(4-pyridyl)ethyl]benzamide |
| 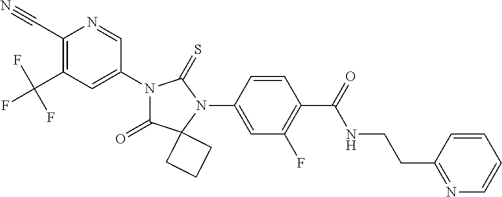 [SMILES: N#Cc1ncc(N2C(=O)C3(CCC3)N(c3ccc(C(=O)NCCc4ccccn4)c(F)c3)C2=S)cc1C(F)(F)F] | 15 | 4-[6-[6-cyano-5-(trifluoromethyl)-3- pyridyl]-5-oxo-7-thioxo-6,8- diazaspiro[3.4]octan-8-yl]-2-fluoro-N- [2-(2-pyridyl)ethyl]benzamide |
| 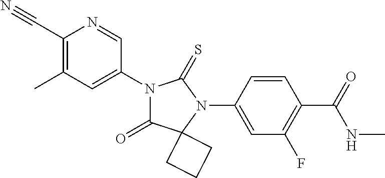 [SMILES: CNC(=O)c1ccc(N2C(=S)N(c3cnc(C#N)c(C)c3)C(=O)C23CCC3)cc1F] | 16 | 4-[6-(6-cyano-5-methyl-3-pyridyl)-5- oxo-7-thioxo-6,8-diazaspiro[3.4]octan- 8-yl]-2-fluoro-N-methyl-benzamide |
| 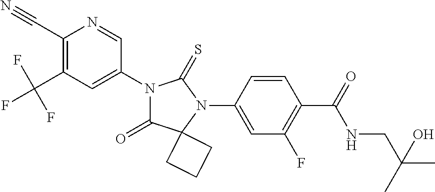 [SMILES: CC(C)(O)CNC(=O)c1ccc(N2C(=S)N(c3cnc(C#N)c(C(F)(F)F)c3)C(=O)C23CCC3)cc1F] | 17 | 4-[6-[6-cyano-5-(trifluoromethyl)-3- pyridyl]-5-oxo-7-thioxo-6,8- diazaspiro[3.4]octan-8-yl]-2-fluoro-N- (2-hydroxy-2-methyl-propyl)benzamide |
| 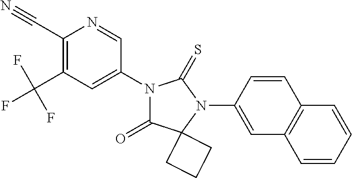 [SMILES: N#Cc1ncc(N2C(=O)C3(CCC3)N(c3ccc4ccccc4c3)C2=S)cc1C(F)(F)F] | 18 | 5-[8-(2-naphthyl)-5-oxo-7-thioxo-6,8- diazaspiro[3.4]octan-6-yl]-3- (trifluoromethyl)pyridine-2-carbonitrile |
| 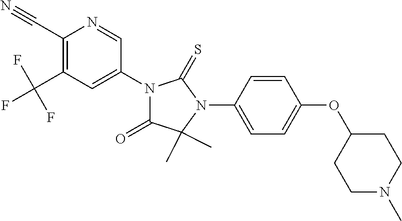 [SMILES: CN1CCC(Oc2ccc(N3C(=S)N(c4cnc(C#N)c(C(F)(F)F)c4)C(=O)C3(C)C)cc2)CC1] | 19 | 5-[4,4-dimethyl-3-[4-[(1-methyl-4- piperidyl)oxy]phenyl]-5-oxo-2-thioxo- imidazolidin-1-yl]-3- (trifluoromethyl)pyridine-2-carbonitrile |
| 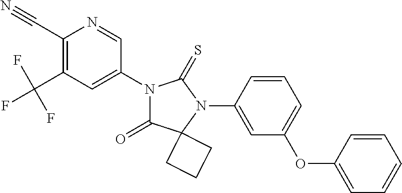 [SMILES: N#Cc1ncc(N2C(=O)C3(CCC3)N(c3cccc(Oc4ccccc4)c3)C2=S)cc1C(F)(F)F] | 20 | 5-[5-oxo-8-(3-phenoxyphenyl)-7- thioxo-6,8-diazaspiro[3.4]octan-6-yl]- 3-(trifluoromethyl)pyridine-2- carbonitrile |
| 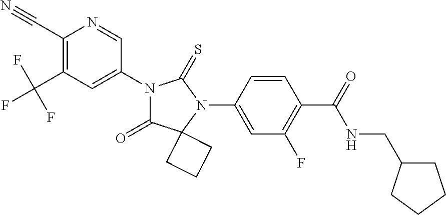 [SMILES: N#Cc1ncc(N2C(=O)C3(CCC3)N(c3ccc(C(=O)NCC4CCCC4)c(F)c3)C2=S)cc1C(F)(F)F] | 21 | 4-[6-[6-cyano-5-(trifluoromethyl)-3- pyridyl]-5-oxo-7-thioxo-6,8- diazaspiro[3.4]octan-8-yl]-N- (cyclopentylmethyl)-2-fluoro- benzamide |
| 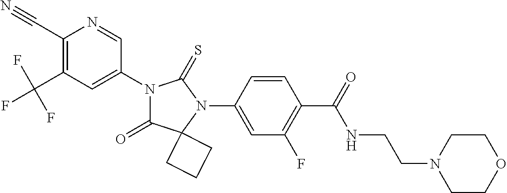 [SMILES: N#Cc1ncc(N2C(=O)C3(CCC3)N(c3ccc(C(=O)NCCN4CCOCC4)c(F)c3)C2=S)cc1C(F)(F)F] | 22 | 4-[6-[6-cyano-5-(trifluoromethyl)-3- pyridyl]-5-oxo-7-thioxo-6,8- diazaspiro[3.4]octan-8-yl]-2-fluoro-N- (2-morpholinoethyl)benzamide |
| 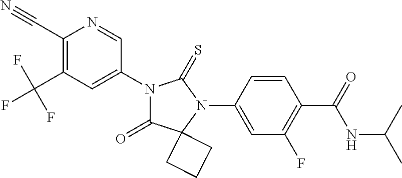 [SMILES: CC(C)NC(=O)c1ccc(N2C(=S)N(c3cnc(C#N)c(C(F)(F)F)c3)C(=O)C23CCC3)cc1F] | 23 | 4-[6-[6-cyano-5-(trifluoromethyl)-3- pyridyl]-5-oxo-7-thioxo-6,8- diazaspiro[3.4]octan-8-yl]-2-fluoro-N- isopropyl-benzamide |
| 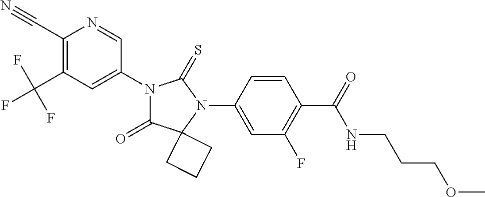 [SMILES: COCCCNC(=O)c1ccc(N2C(=S)N(c3cnc(C#N)c(C(F)(F)F)c3)C(=O)C23CCC3)cc1F] | 24 | 4-[6-[6-cyano-5-(trifluoromethyl)-3- pyridyl]-5-oxo-7-thioxo-6,8- diazaspiro[3.4]octan-8-yl]-2-fluoro-N- (3-methoxypropyl)benzamide |
| 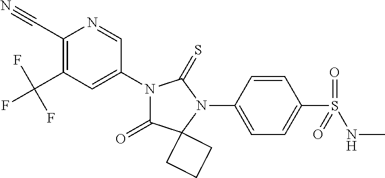 [SMILES: CNS(=O)(=O)c1ccc(N2C(=S)N(c3cnc(C#N)c(C(F)(F)F)c3)C(=O)C23CCC3)cc1] | 25 | 4-[6-[6-cyano-5-(trifluoromethyl)-3- pyridyl]-5-oxo-7-thioxo-6,8- diazaspiro[3.4]octan-8-yl]-N-methyl- benzenesulfonamide |
| 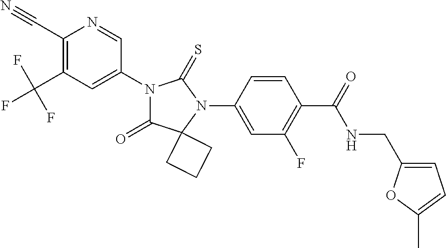 [SMILES: Cc1ccc(CNC(=O)c2ccc(N3C(=S)N(c4cnc(C#N)c(C(F)(F)F)c4)C(=O)C34CCC4)cc2F)o1] | 26 | 4-[6-[6-cyano-5-(trifluoromethyl)-3- pyridyl]-5-oxo-7-thioxo-6,8- diazaspiro[3.4]octan-8-yl]-2-fluoro-N- [(5-methyl-2-furyl)methyl]benzamide |
| 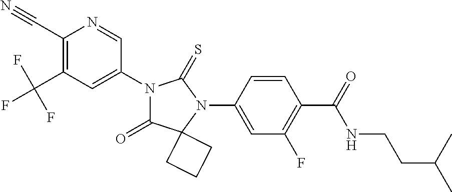 [SMILES: CC(C)CCNC(=O)c1ccc(N2C(=S)N(c3cnc(C#N)c(C(F)(F)F)c3)C(=O)C23CCC3)cc1F] | 27 | 4-[6-[6-cyano-5-(trifluoromethyl)-3- pyridyl]-5-oxo-7-thioxo-6,8- diazaspiro[3.4]octan-8-yl]-2-fluoro-N- isopentyl-benzamide |
| 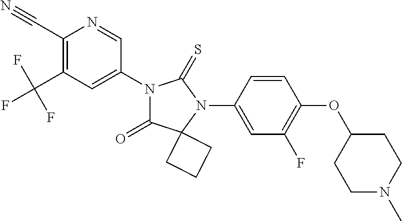 [SMILES: CN1CCC(Oc2ccc(N3C(=S)N(c4cnc(C#N)c(C(F)(F)F)c4)C(=O)C34CCC4)cc2F)CC1] | 28 | 5-[8-[3-fluoro-4-[(1-methyl-4- piperidyl)oxy]phenyl]-5-oxo-7-thioxo- 6,8-diazaspiro[3.4]octan-6-yl]-3- (trifluoromethyl)pyridine-2-carbonitrile |
| 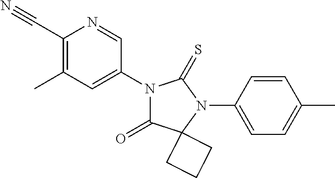 [SMILES: Cc1ccc(N2C(=S)N(c3cnc(C#N)c(C)c3)C(=O)C23CCC3)cc1] | 29 | 3-methyl-5-[5-oxo-8-(p-tolyl)-7-thioxo- 6,8-diazaspiro[3.4]octan-6-yl]pyridine- 2-carbonitrile |
| 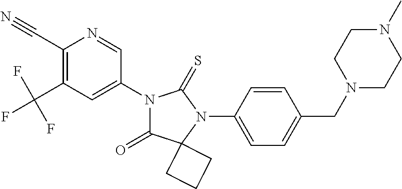 [SMILES: CN1CCN(Cc2ccc(N3C(=S)N(c4cnc(C#N)c(C(F)(F)F)c4)C(=O)C34CCC4)cc2)CC1] | 30 | 5-[8-[4-[(4-methylpiperazin-1- yl)methyl]phenyl]-5-oxo-7-thioxo-6,8- diazaspiro[3.4]octan-6-yl]-3- (trifluoromethyl)pyridine-2-carbonitrile |
| 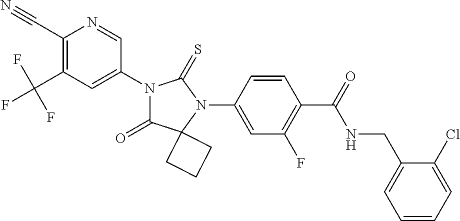 [SMILES: N#Cc1ncc(N2C(=O)C3(CCC3)N(c3ccc(C(=O)NCc4ccccc4Cl)c(F)c3)C2=S)cc1C(F)(F)F] | 31 | N-[(2-chlorophenyl)methyl]-4-[6-[6- cyano-5-(trifluoromethyl)-3-pyridyl]-5- oxo-7-thioxo-6,8-diazaspiro[3.4]octan- 8-yl]-2-fluoro-benzamide |
| 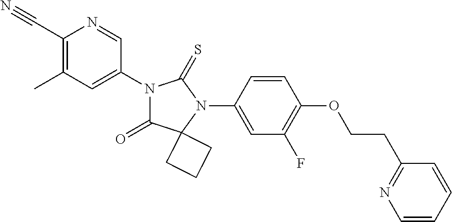 [SMILES: Cc1cc(N2C(=O)C3(CCC3)N(c3ccc(OCCc4ccccn4)c(F)c3)C2=S)cnc1C#N] | 32 | 5-[8-[3-fluoro-4-[2-(2- pyridyl)ethoxy]phenyl]-5-oxo-7-thioxo- 6,8-diazaspiro[3.4]octan-6-yl]-3- methyl-pyridine-2-carbonitrile |
| 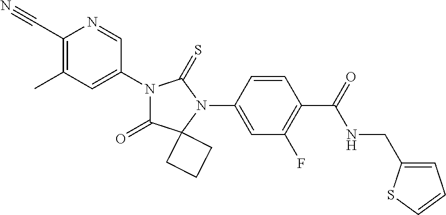 [SMILES: Cc1cc(N2C(=O)C3(CCC3)N(c3ccc(C(=O)NCc4cccs4)c(F)c3)C2=S)cnc1C#N] | 33 | 4-[6-(6-cyano-5-methyl-3-pyridyl)-5- oxo-7-thioxo-6,8-diazaspiro[3.4]octan- 8-yl]-2-fluoro-N-(2- thienylmethyl)benzamide |
| 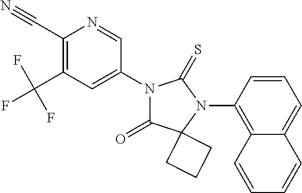 [SMILES: N#Cc1ncc(N2C(=O)C3(CCC3)N(c3cccc4ccccc34)C2=S)cc1C(F)(F)F] | 34 | 5-[8-(1-naphthyl)-5-oxo-7-thioxo-6,8- diazaspiro[3.4]octan-6-yl]-3- (trifluoromethyl)pyridine-2-carbonitrile |
| 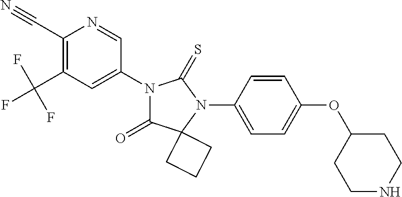 [SMILES: N#Cc1ncc(N2C(=O)C3(CCC3)N(c3ccc(OC4CCNCC4)cc3)C2=S)cc1C(F)(F)F] | 35 | 5-[5-oxo-8-[4-(4-piperidyloxy)phenyl]- 7-thioxo-6,8-diazaspiro[3.4]octan-6- yl]-3-(trifluoromethyl)pyridine-2- carbonitrile |
| 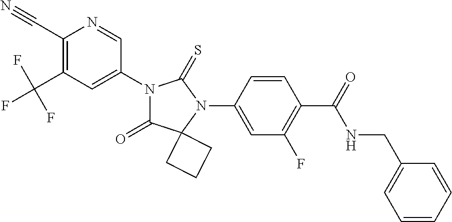 [SMILES: N#Cc1ncc(N2C(=O)C3(CCC3)N(c3ccc(C(=O)NCc4ccccc4)c(F)c3)C2=S)cc1C(F)(F)F] | 36 | N-benzyl-4-[6-[6-cyano-5- (trifluoromethyl)-3-pyridyl]-5-oxo-7- thioxo-6,8-diazaspiro[3.4]octan-8-yl]- 2-fluoro-benzamide |
| 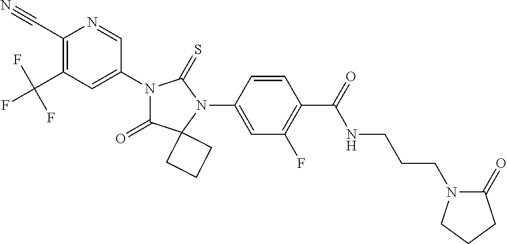 [SMILES: N#Cc1ncc(N2C(=O)C3(CCC3)N(c3ccc(C(=O)NCCCN4CCCC4=O)c(F)c3)C2=S)cc1C(F)(F)F] | 37 | 4-[6-[6-cyano-5-(trifluoromethyl)-3- pyridyl]-5-oxo-7-thioxo-6,8- diazaspiro[3.4]octan-8-yl]-2-fluoro-N- [3-(2-oxopyrrolidin-1- yl)propyl]benzamide |
| 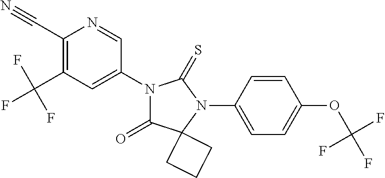 [SMILES: N#Cc1ncc(N2C(=O)C3(CCC3)N(c3ccc(OC(F)(F)F)cc3)C2=S)cc1C(F)(F)F] | 38 | 5-[5-oxo-7-thioxo-8-[4- (trifluoromethoxy)phenyl]-6,8- diazaspiro[3.4]octan-6-yl]-3- (trifluoromethyl)pyridine-2-carbonitrile |
| 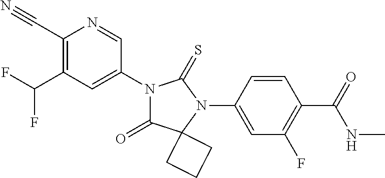 [SMILES: CNC(=O)c1ccc(N2C(=S)N(c3cnc(C#N)c(C(F)F)c3)C(=O)C23CCC3)cc1F] | 39 | 4-[6-[6-cyano-5-(difluoromethyl)-3- pyridyl]-5-oxo-7-thioxo-6,8- diazaspiro[3.4]octan-8-yl]-2-fluoro-N- methyl-benzamide |
| 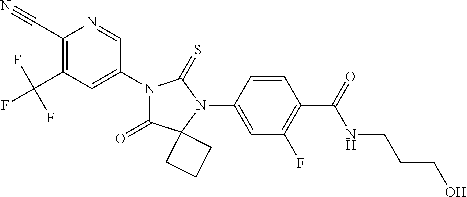 [SMILES: N#Cc1ncc(N2C(=O)C3(CCC3)N(c3ccc(C(=O)NCCCO)c(F)c3)C2=S)cc1C(F)(F)F] | 40 | 4-[6-[6-cyano-5-(trifluoromethyl)-3- pyridyl]-5-oxo-7-thioxo-6,8- diazaspiro[3.4]octan-8-yl]-2-fluoro-N- (3-hydroxypropyl)benzamide |
| 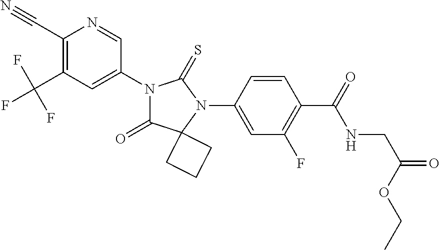 [SMILES: CCOC(=O)CNC(=O)c1ccc(N2C(=S)N(c3cnc(C#N)c(C(F)(F)F)c3)C(=O)C23CCC3)cc1F] | 41 | ethyl 2-[[4-[6-[6-cyano-5- (trifluoromethyl)-3-pyridyl]-5-oxo-7- thioxo-6,8-diazaspiro[3.4]octan-8-yl]- 2-fluoro-benzoyl]amino]acetate |
| 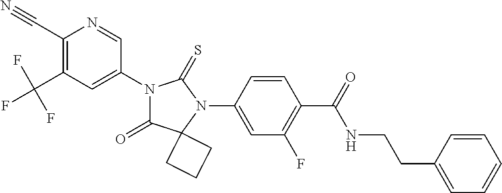 [SMILES: N#Cc1ncc(N2C(=O)C3(CCC3)N(c3ccc(C(=O)NCCc4ccccc4)c(F)c3)C2=S)cc1C(F)(F)F] | 42 | 4-[6-[6-cyano-5-(trifluoromethyl)-3- pyridyl]-5-oxo-7-thioxo-6,8- diazaspiro[3.4]octan-8-yl]-2-fluoro-N- phenethyl-benzamide |
| 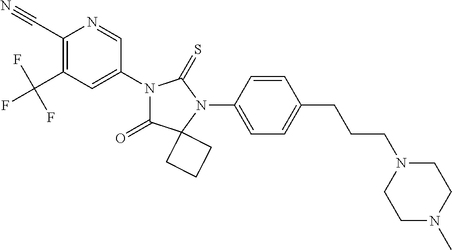 [SMILES: CN1CCN(CCCc2ccc(N3C(=S)N(c4cnc(C#N)c(C(F)(F)F)c4)C(=O)C34CCC4)cc2)CC1] | 43 | 5-[8-[4-[3-(4-methylpiperazin-1- yl)propyl]phenyl]-5-oxo-7-thioxo-6,8- diazaspiro[3.4]octan-6-yl]-3- (trifluoromethyl)pyridine-2-carbonitrile |
| 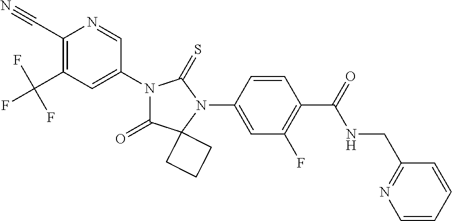 [SMILES: N#Cc1ncc(N2C(=O)C3(CCC3)N(c3ccc(C(=O)NCc4ccccn4)c(F)c3)C2=S)cc1C(F)(F)F] | 44 | 4-[6-[6-cyano-5-(trifluoromethyl)-3- pyridyl]-5-oxo-7-thioxo-6,8- diazaspiro[3.4]octan-8-yl]-2-fluoro-N- (2-pyridylmethyl)benzamide |
| 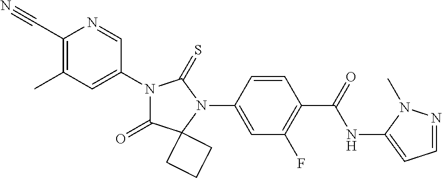 [SMILES: Cc1cc(N2C(=O)C3(CCC3)N(c3ccc(C(=O)Nc4ccnn4C)c(F)c3)C2=S)cnc1C#N] | 45 | 4-[6-(6-cyano-5-methyl-3-pyridyl)-5- oxo-7-thioxo-6,8-diazaspiro[3.4]octan- 8-yl]-2-fluoro-N-(2-methylpyrazol-3- yl)benzamide |
| 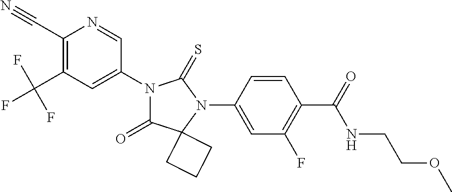 [SMILES: COCCNC(=O)c1ccc(N2C(=S)N(c3cnc(C#N)c(C(F)(F)F)c3)C(=O)C23CCC3)cc1F] | 46 | 4-[6-[6-cyano-5-(trifluoromethyl)-3- pyridyl]-5-oxo-7-thioxo-6,8- diazaspiro[3.4]octan-8-yl]-2-fluoro-N- (2-methoxyethyl)benzamide |
| 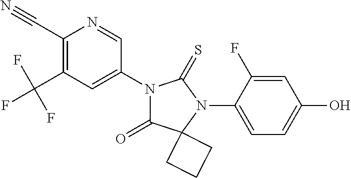 [SMILES: N#Cc1ncc(N2C(=O)C3(CCC3)N(c3ccc(O)cc3F)C2=S)cc1C(F)(F)F] | 47 | 5-[8-(2-fluoro-4-hydroxy-phenyl)-5- oxo-7-thioxo-6,8-diazaspiro[3.4]octan- 6-yl]-3-(trifluoromethyl)pyridine-2- carbonitrile |
| 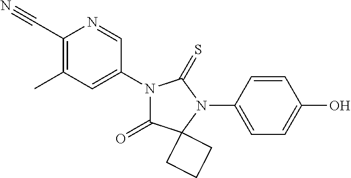 [SMILES: Cc1cc(N2C(=O)C3(CCC3)N(c3ccc(O)cc3)C2=S)cnc1C#N] | 48 | 5-[8-(4-hydroxyphenyl)-5-oxo-7- thioxo-6,8-diazaspiro[3.4]octan-6-yl]- 3-methyl-pyridine-2-carbonitrile |
| 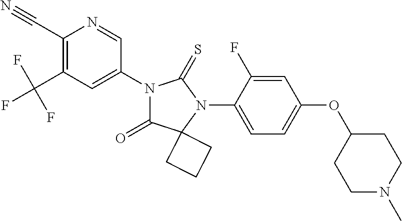 [SMILES: CN1CCC(Oc2ccc(N3C(=S)N(c4cnc(C#N)c(C(F)(F)F)c4)C(=O)C34CCC4)c(F)c2)CC1] | 49 | 5-[8-[2-fluoro-4-[(1-methyl-4- piperidyl)oxy]phenyl]-5-oxo-7-thioxo- 6,8-diazaspiro[3.4]octan-6-yl]-3- (trifluoromethyl)pyridine-2-carbonitrile |
| 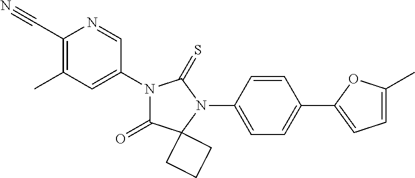 [SMILES: Cc1ccc(-c2ccc(N3C(=S)N(c4cnc(C#N)c(C)c4)C(=O)C34CCC4)cc2)o1] | 50 | 3-methyl-5-[8-[4-(5-methyl-2- furyl)phenyl]-5-oxo-7-thioxo-6,8- diazaspiro[3.4]octan-6-yl]pyridine-2- carbonitrile |
| 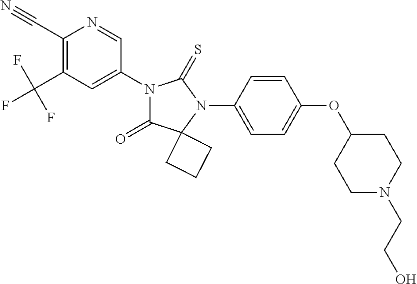 [SMILES: N#Cc1ncc(N2C(=O)C3(CCC3)N(c3ccc(OC4CCN(CCO)CC4)cc3)C2=S)cc1C(F)(F)F] | 51 | 5-[8-[4-[[1-(2-hydroxyethyl)-4- piperidyl]oxy]phenyl]-5-oxo-7-thioxo- 6,8-diazaspiro[3.4]octan-6-yl]-3- (trifluoromethyl)pyridine-2-carbonitrile |
| 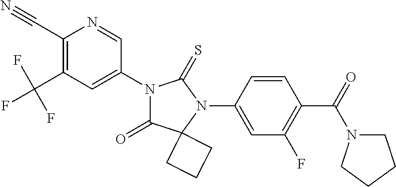 [SMILES: N#Cc1ncc(N2C(=O)C3(CCC3)N(c3ccc(C(=O)N4CCCC4)c(F)c3)C2=S)cc1C(F)(F)F] | 52 | 5-[8-[3-fluoro-4-(pyrrolidine-1- carbonyl)phenyl]-5-oxo-7-thioxo-6,8- diazaspiro[3.4]octan-6-yl]-3- (trifluoromethyl)pyridine-2-carbonitrile |
| 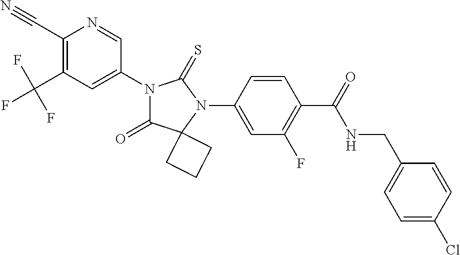 [SMILES: N#Cc1ncc(N2C(=O)C3(CCC3)N(c3ccc(C(=O)NCc4ccc(Cl)cc4)c(F)c3)C2=S)cc1C(F)(F)F] | 53 | N-[(4-chlorophenyl)methyl]-4-[6-[6- cyano-5-(trifluoromethyl)-3-pyridyl]-5- oxo-7-thioxo-6,8-diazaspiro[3.4]octan- 8-yl]-2-fluoro-benzamide |
| 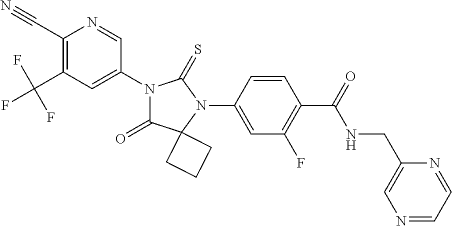 [SMILES: N#Cc1ncc(N2C(=O)C3(CCC3)N(c3ccc(C(=O)NCc4cnccn4)c(F)c3)C2=S)cc1C(F)(F)F] | 54 | 4-[6-[6-cyano-5-(trifluoromethyl)-3- pyridyl]-5-oxo-7-thioxo-6,8- diazaspiro[3.4]octan-8-yl]-2-fluoro-N- (pyrazin-2-ylmethyl)benzamide |
| 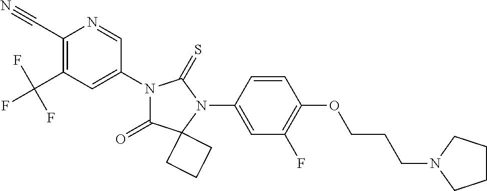 [SMILES: N#Cc1ncc(N2C(=O)C3(CCC3)N(c3ccc(OCCCN4CCCC4)c(F)c3)C2=S)cc1C(F)(F)F] | 55 | 5-[8-[3-fluoro-4-(3-pyrrolidin-1- ylpropoxy)phenyl]-5-oxo-7-thioxo-6,8- diazaspiro[3.4]octan-6-yl]-3- (trifluoromethyl)pyridine-2-carbonitrile |
| 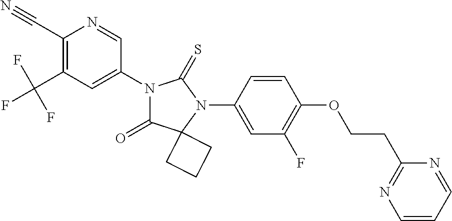 [SMILES: N#Cc1ncc(N2C(=O)C3(CCC3)N(c3ccc(OCCc4ncccn4)c(F)c3)C2=S)cc1C(F)(F)F] | 56 | 5-[8-[3-fluoro-4-(2-pyrimidin-2- ylethoxy)phenyl]-5-oxo-7-thioxo-6,8- diazaspiro[3.4]octan-6-yl]-3- (trifluoromethyl)pyridine-2-carbonitrile |
| 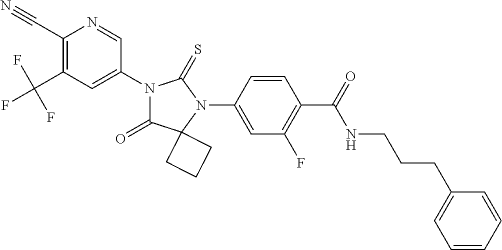 [SMILES: N#Cc1ncc(N2C(=O)C3(CCC3)N(c3ccc(C(=O)NCCCc4ccccc4)c(F)c3)C2=S)cc1C(F)(F)F] | 57 | 4-[6-[6-cyano-5-(trifluoromethyl)-3- pyridyl]-5-oxo-7-thioxo-6,8- diazaspiro[3.4]octan-8-yl]-2-fluoro-N- (3-phenylpropyl)benzamide |
| 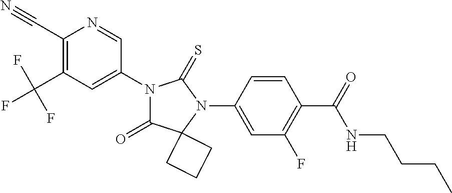 [SMILES: CCCCNC(=O)c1ccc(N2C(=S)N(c3cnc(C#N)c(C(F)(F)F)c3)C(=O)C23CCC3)cc1F] | 58 | N-butyl-4-[6-[6-cyano-5- (trifluoromethyl)-3-pyridyl]-5-oxo-7- thioxo-6,8-diazaspiro[3.4]octan-8-yl]- 2-fluoro-benzamide |
| 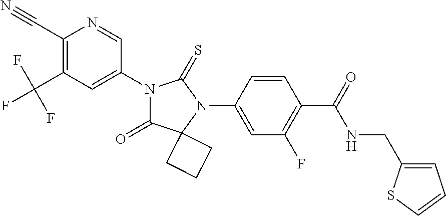 [SMILES: N#Cc1ncc(N2C(=O)C3(CCC3)N(c3ccc(C(=O)NCc4cccs4)c(F)c3)C2=S)cc1C(F)(F)F] | 59 | 4-[6-[6-cyano-5-(trifluoromethyl)-3- pyridyl]-5-oxo-7-thioxo-6,8- diazaspiro[3.4]octan-8-yl]-2-fluoro-N- (2-thienylmethyl)benzamide |
| 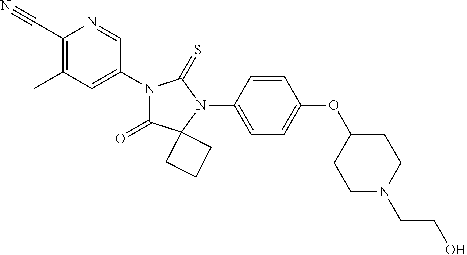 [SMILES: Cc1cc(N2C(=O)C3(CCC3)N(c3ccc(OC4CCN(CCO)CC4)cc3)C2=S)cnc1C#N] | 60 | 5-[8-[4-[[1-(2-hydroxyethyl)-4- piperidyl]oxy]phenyl]-5-oxo-7-thioxo- 6,8-diazaspiro[3.4]octan-6-yl]-3- methyl-pyridine-2-carbonitrile |
| 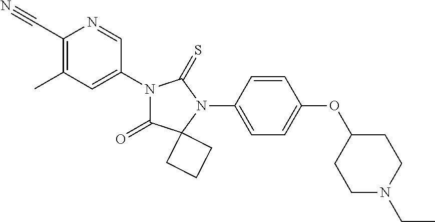 [SMILES: CCN1CCC(Oc2ccc(N3C(=S)N(c4cnc(C#N)c(C)c4)C(=O)C34CCC4)cc2)CC1] | 61 | 5-[8-[4-[(1-ethyl-4- piperidyl)oxy]phenyl]-5-oxo-7-thioxo- 6,8-diazaspiro[3.4]octan-6-yl]-3- methyl-pyridine-2-carbonilrile |
| 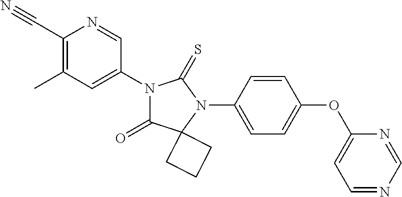 [SMILES: Cc1cc(N2C(=O)C3(CCC3)N(c3ccc(Oc4ccncn4)cc3)C2=S)cnc1C#N] | 62 | 3-methyl-5-[5-oxo-8-(4-pyrimidin-4- yloxyphenyl)-7-thioxo-6,8- diazaspiro[3.4]octan-6-yl]pyridine-2- carbonitrile |
| 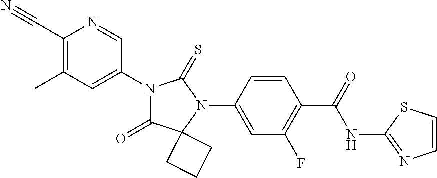 [SMILES: Cc1cc(N2C(=O)C3(CCC3)N(c3ccc(C(=O)Nc4nccs4)c(F)c3)C2=S)cnc1C#N] | 63 | 4-[6-(6-cyano-5-methyl-3-pyridyl)-5- oxo-7-thioxo-6,8-diazaspiro[3.4]octan- 8-yl]-2-fluoro-N-thiazol-2-yl- benzamide |
| 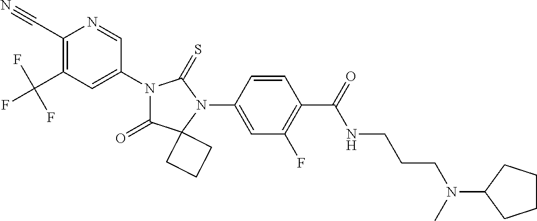 [SMILES: CN(CCCNC(=O)c1ccc(N2C(=S)N(c3cnc(C#N)c(C(F)(F)F)c3)C(=O)C23CCC3)cc1F)C1CCCC1] | 64 | 4-[6-[6-cyano-5-(trifluoromethyl)-3- pyridyl]-5-oxo-7-thioxo-6,8- diazaspiro[3.4]octan-8-yl]-N-[3- [cyclopentyl(methyl)amino]propyl]-2- fluoro-benzamide |
| 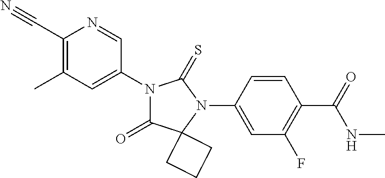 [SMILES: CNC(=O)c1ccc(N2C(=S)N(c3cnc(C#N)c(C)c3)C(=O)C23CCC3)cc1F] | 65 | 4-[7-(6-cyano-5-methyl-3-pyridyl)-6- oxo-8-thioxo-7,9-diazaspiro[4.4]nonan- 9-yl]-2-fluoro-N-methyl-benzamide |
| 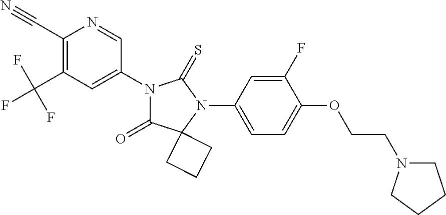 [SMILES: N#Cc1ncc(N2C(=O)C3(CCC3)N(c3ccc(OCCN4CCCC4)c(F)c3)C2=S)cc1C(F)(F)F] | 66 | 5-[8-[3-fluoro-4-(2-pyrrolidin-1- ylethoxy)phenyl]-5-oxo-7-thioxo-6,8- diazaspiro[3.4]octan-6-yl]-3- (trifluoromethyl)pyridine-2-carbonitrile |
| 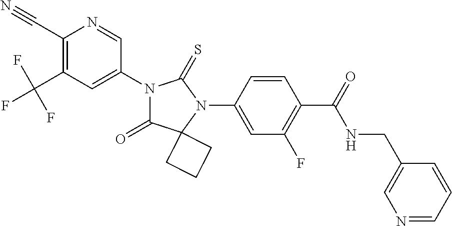 [SMILES: N#Cc1ncc(N2C(=O)C3(CCC3)N(c3ccc(C(=O)NCc4cccnc4)c(F)c3)C2=S)cc1C(F)(F)F] | 67 | 4-[6-[6-cyano-5-(trifluoromethyl)-3- pyridyl]-5-oxo-7-thioxo-6,8- diazaspiro[3.4]octan-8-yl]-2-fluoro-N- (3-pyridylmethyl)benzamide |
| 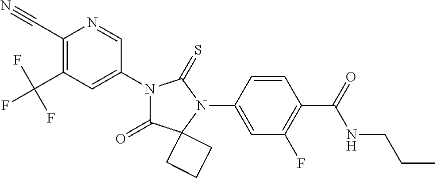 [SMILES: CCCNC(=O)c1ccc(N2C(=S)N(c3cnc(C#N)c(C(F)(F)F)c3)C(=O)C23CCC3)cc1F] | 68 | 4-[6-[6-cyano-5-(trifluoromethyl)-3- pyridyl]-5-oxo-7-thioxo-6,8- diazaspiro[3.4]octan-8-yl]-2-fluoro-N- propyl-benzamide |
| 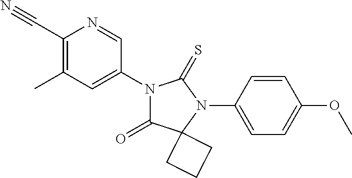 [SMILES: COc1ccc(N2C(=S)N(c3cnc(C#N)c(C)c3)C(=O)C23CCC3)cc1] | 69 | 5-[8-(4-methoxyphenyl)-5-oxo-7- thioxo-6,8-diazaspiro[3.4]octan-6-yl]- 3-methyl-pyridine-2-carbonitrile |
| 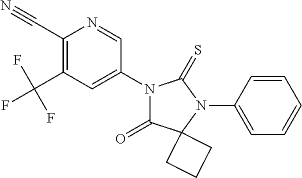 [SMILES: N#Cc1ncc(N2C(=O)C3(CCC3)N(c3ccccc3)C2=S)cc1C(F)(F)F] | 70 | 5-(5-oxo-8-phenyl-7-thioxo-6,8- diazaspiro[3.4]octan-6-yl)-3- (trifluoromethyl)pyridine-2-carbonitrile |
| 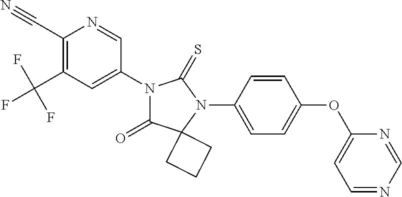 [SMILES: N#Cc1ncc(N2C(=O)C3(CCC3)N(c3ccc(Oc4ccncn4)cc3)C2=S)cc1C(F)(F)F] | 71 | 5-[5-oxo-8-(4-pyrimidin-4- yloxyphenyl)-7-thioxo-6,8- diazaspiro[3.4]octan-6-yl]-3- (trifluoromethyl)pyridine-2-carbonitrile |
| 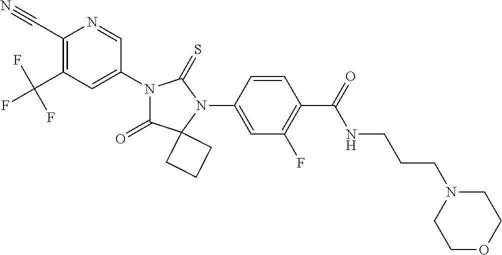 [SMILES: N#Cc1ncc(N2C(=O)C3(CCC3)N(c3ccc(C(=O)NCCCN4CCOCC4)c(F)c3)C2=S)cc1C(F)(F)F] | 72 | 4-[6-[6-cyano-5-(trifluoromethyl)-3- pyridyl]-5-oxo-7-thioxo-6,8- diazaspiro[3.4]octan-8-yl]-2-fluoro-N- (3-morpholinopropyl)benzamide |
| 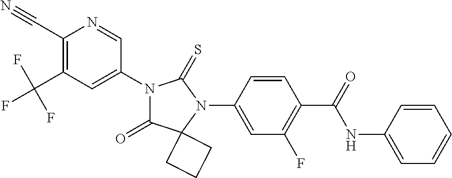 [SMILES: N#Cc1ncc(N2C(=O)C3(CCC3)N(c3ccc(C(=O)Nc4ccccc4)c(F)c3)C2=S)cc1C(F)(F)F] | 73 | 4-[6-[6-cyano-5-(trifluoromethyl)-3- pyridyl]-5-oxo-7-thioxo-6,8- diazaspiro[3.4]octan-8-yl]-2-fluoro-N- phenyl-benzamide |
| 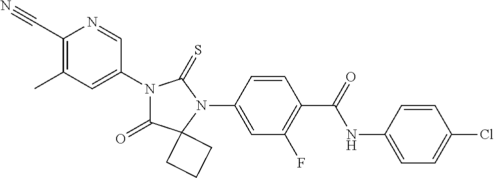 [SMILES: Cc1cc(N2C(=O)C3(CCC3)N(c3ccc(C(=O)Nc4ccc(Cl)cc4)c(F)c3)C2=S)cnc1C#N] | 74 | N-(4-chlorophenyl)-4-[6-(6-cyano-5- methyl-3-pyridyl)-5-oxo-7-thioxo-6,8- diazaspiro[3.4]octan-8-yl]-2-fluoro- benzamide |
| 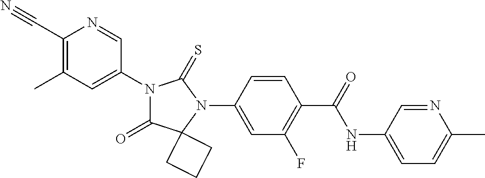 [SMILES: Cc1ccc(NC(=O)c2ccc(N3C(=S)N(c4cnc(C#N)c(C)c4)C(=O)C34CCC4)cc2F)cn1] | 75 | 4-[6-(6-cyano-5-methyl-3-pyridyl)-5- oxo-7-thioxo-6,8-diazaspiro[3.4]octan- 8-yl]-2-fluoro-N-(6-methyl-3- pyridyl)benzamide |
| 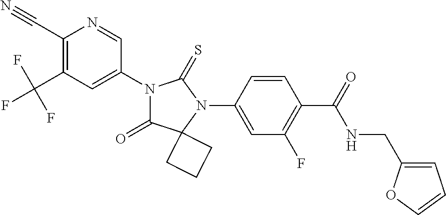 [SMILES: N#Cc1ncc(N2C(=O)C3(CCC3)N(c3ccc(C(=O)NCc4ccco4)c(F)c3)C2=S)cc1C(F)(F)F] | 76 | 4-[6-[6-cyano-5-(trifluoromethyl)-3- pyridyl]-5-oxo-7-thioxo-6,8- diazaspiro[3.4]octan-8-yl]-2-fluoro-N- (2-furylmethyl)benzamide |
| 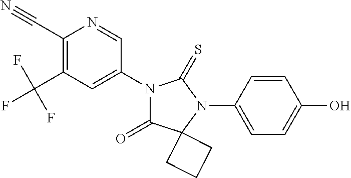 [SMILES: N#Cc1ncc(N2C(=O)C3(CCC3)N(c3ccc(O)cc3)C2=S)cc1C(F)(F)F] | 77 | 5-[8-(4-hydroxyphenyl)-5-oxo-7- thioxo-6,8-diazaspiro[3.4]octan-6-yl]- 3-(trifluoromethyl)pyridine-2- carbonitrile |
| 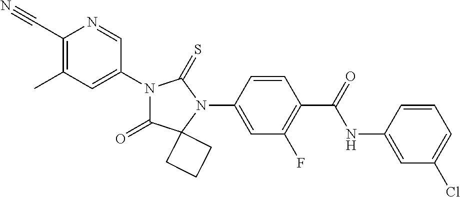 [SMILES: Cc1cc(N2C(=O)C3(CCC3)N(c3ccc(C(=O)Nc4cccc(Cl)c4)c(F)c3)C2=S)cnc1C#N] | 78 | N-(3-chlorophenyl)-4-[6-(6-cyano-5- methyl-3-pyridyl)-5-oxo-7-thioxo-6,8- diazaspiro[3.4]octan-8-yl]-2-fluoro- benzamide |
| 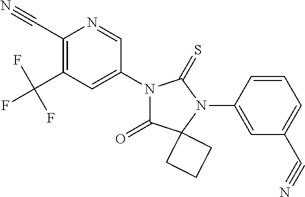 [SMILES: N#Cc1cccc(N2C(=S)N(c3cnc(C#N)c(C(F)(F)F)c3)C(=O)C23CCC3)c1] | 79 | 5-[8-(3-cyanophenyl)-5-oxo-7-thioxo- 6,8-diazaspiro[3.4]octan-6-yl]-3- (trifluoromethyl)pyridine-2-carbonitrile |
| 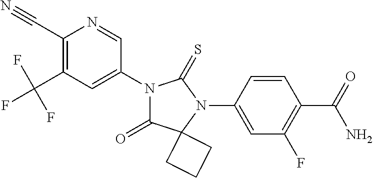 [SMILES: N#Cc1ncc(N2C(=O)C3(CCC3)N(c3ccc(C(N)=O)c(F)c3)C2=S)cc1C(F)(F)F] | 80 | 4-[6-[6-cyano-5-(trifluoromethyl)-3- pyridyl]-5-oxo-7-thioxo-6,8- diazaspiro[3.4]octan-8-yl]-2-fluoro- benzamide |
| 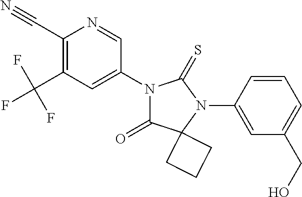 [SMILES: N#Cc1ncc(N2C(=O)C3(CCC3)N(c3cccc(CO)c3)C2=S)cc1C(F)(F)F] | 81 | 5-[8-[3-(hydroxymethyl)phenyl]-5-oxo- 7-thioxo-6,8-diazaspiro[3.4]octan-6- yl]-3-(trifluoromethyl)pyridine-2- carbonitrile |
| 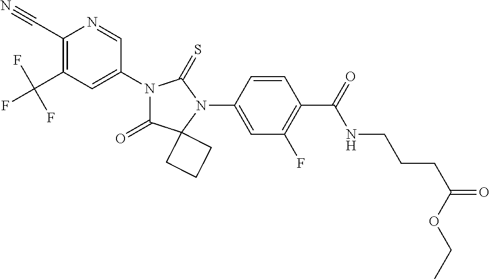 [SMILES: CCOC(=O)CCCNC(=O)c1ccc(N2C(=S)N(c3cnc(C#N)c(C(F)(F)F)c3)C(=O)C23CCC3)cc1F] | 82 | ethyl 4-[[4-[6-[6-cyano-5- (trifluoromethyl)-3-pyridyl]-5-oxo-7- thioxo-6,8-diazaspiro[3.4]octan-8-yl]- 2-fluoro-benzoyl]amino]butanoate |
| 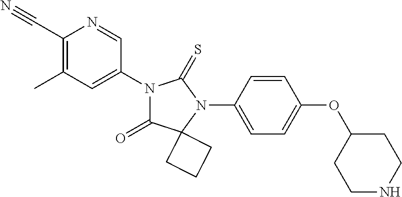 [SMILES: Cc1cc(N2C(=O)C3(CCC3)N(c3ccc(OC4CCNCC4)cc3)C2=S)cnc1C#N] | 83 | 3-methyl-5-[5-oxo-8-[4-(4- piperidyloxy)phenyl]-7-thioxo-6,8- diazaspiro[3.4]octan-6-yl]pyridine-2- carbonitrile |
| 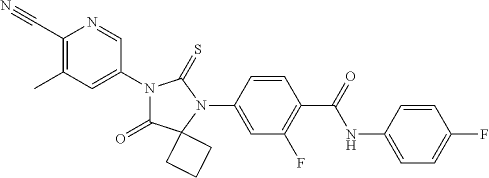 [SMILES: Cc1cc(N2C(=O)C3(CCC3)N(c3ccc(C(=O)Nc4ccc(F)cc4)c(F)c3)C2=S)cnc1C#N] | 84 | 4-[6-(6-cyano-5-methyl-3-pyridyl)-5- oxo-7-thioxo-6,8-diazaspiro[3.4]octan- 8-yl]-2-fluoro-N-(4- fluorophenyl)benzamide |
| 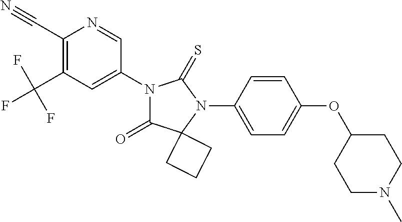 [SMILES: CN1CCC(Oc2ccc(N3C(=S)N(c4cnc(C#N)c(C(F)(F)F)c4)C(=O)C34CCC4)cc2)CC1] | 85 | 5-[8-[4-[(1-methyl-4- piperidyl)oxy]phenyl]-5-oxo-7-thioxo- 6,8-diazaspiro[3.4]octan-6-yl]-3- (trifluoromethyl)pyridine-2-carbonitrile |
| 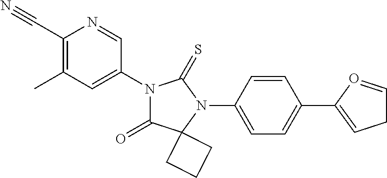 | 86 | 5-[8-[4-(2-furyl)phenyl]-5-oxo-7- thioxo-6,8-diazaspiro[3.4]octan-6-yl]- 3-methyl-pyridine-2-carbonitrile |
| 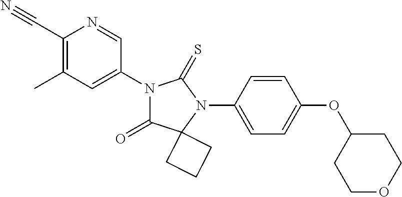 [SMILES: Cc1cc(N2C(=O)C3(CCC3)N(c3ccc(OC4CCOCC4)cc3)C2=S)cnc1C#N] | 87 | 3-methyl-5-[5-oxo-8-(4- tetrahydropyran-4-yloxyphenyl)-7- thioxo-6,8-diazaspiro[3.4]octan-6- yl]pyridine-2-carbonitrile |
| 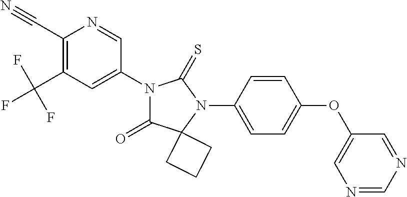 [SMILES: N#Cc1ncc(N2C(=O)C3(CCC3)N(c3ccc(Oc4cncnc4)cc3)C2=S)cc1C(F)(F)F] | 88 | 5-[5-oxo-8-(4-pyrimidin-5- yloxyphenyl)-7-thioxo-6,8- diazaspiro[3.4]octan-6-yl]-3- (trifluoromethyl)pyridine-2-carbonitrile |
| 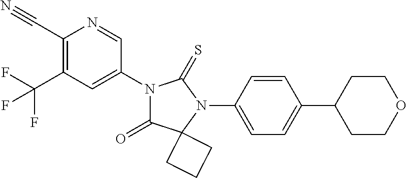 [SMILES: N#Cc1ncc(N2C(=O)C3(CCC3)N(c3ccc(C4CCOCC4)cc3)C2=S)cc1C(F)(F)F] | 89 | 5-[5-oxo-8-(4-tetrahydropyran-4- ylphenyl)-7-thioxo-6,8- diazaspiro[3.4]octan-6-yl]-3- (trifluoromethyl)pyridine-2-carbonitrile |
| 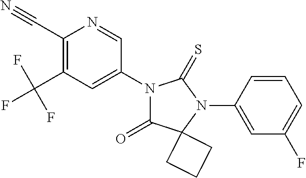 [SMILES: N#Cc1ncc(N2C(=O)C3(CCC3)N(c3cccc(F)c3)C2=S)cc1C(F)(F)F] | 90 | 5-[8-(3-fluorophenyl)-5-oxo-7-thioxo- 6,8-diazaspiro[3.4]octan-6-yl]-3- (trifluoromethyl)pyridine-2-carbonitrile |
| 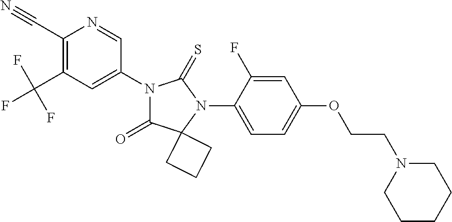 [SMILES: N#Cc1ncc(N2C(=O)C3(CCC3)N(c3ccc(OCCN4CCCCC4)cc3F)C2=S)cc1C(F)(F)F] | 91 | 5-[8-[2-fluoro-4-[2-(1- piperidyl)ethoxy]phenyl]-5-oxo-7- thioxo-6,8-diazaspiro[3.4]octan-6-yl]- 3-(trifluoromethyl)pyridine-2- carbonitrile |
| 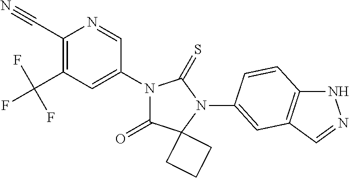 [SMILES: N#Cc1ncc(N2C(=O)C3(CCC3)N(c3ccc4[nH]ncc4c3)C2=S)cc1C(F)(F)F] | 92 | 5-[8-(1H-indazol-5-yl)-5-oxo-7-thioxo- 6,8-diazaspiro[3.4]octan-6-yl]-3- (trifluoromethyl)pyridine-2-carbonitrile |
| 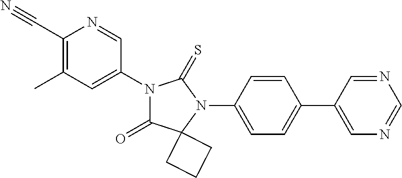 [SMILES: Cc1cc(N2C(=O)C3(CCC3)N(c3ccc(-c4cncnc4)cc3)C2=S)cnc1C#N] | 93 | 3-methyl-5-[5-oxo-8-(4-pyrimidin-5- ylphenyl)-7-thioxo-6,8- diazaspiro[3.4]octan-6-yl]pyridine-2- carbonitrile |
| 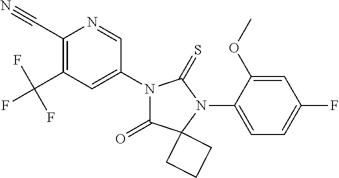 [SMILES: COc1cc(F)ccc1N1C(=S)N(c2cnc(C#N)c(C(F)(F)F)c2)C(=O)C12CCC2] | 94 | 5-[8-(4-fluoro-2-methoxy-phenyl)-5- oxo-7-thioxo-6,8-diazaspiro[3.4]octan- 6-yl]-3-(trifluoromethyl)pyridine-2- carbonitrile |
| 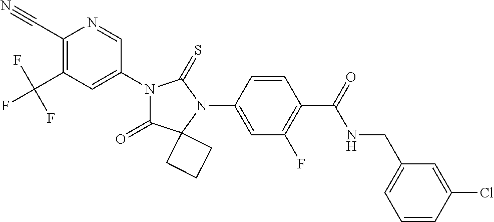 [SMILES: N#Cc1ncc(N2C(=O)C3(CCC3)N(c3ccc(C(=O)NCc4cccc(Cl)c4)c(F)c3)C2=S)cc1C(F)(F)F] | 95 | N-[(3-chlorophenyl)methyl]-4-[6-[6- cyano-5-(trifluoromethyl)-3-pyridyl]-5- oxo-7-thioxo-6,8-diazaspiro[3.4]octan- 8-yl]-2-fluoro-benzamide |
| 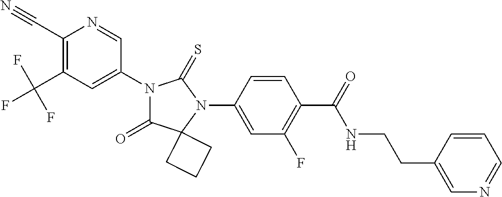 [SMILES: N#Cc1ncc(N2C(=O)C3(CCC3)N(c3ccc(C(=O)NCCc4cccnc4)c(F)c3)C2=S)cc1C(F)(F)F] | 96 | 4-[6-[6-cyano-5-(trifluoromethyl)-3- pyridyl]-5-oxo-7-thioxo-6,8- diazaspiro[3.4]octan-8-yl]-2-fluoro-N- [2-(3-pyridyl)ethyl]benzamide |
| 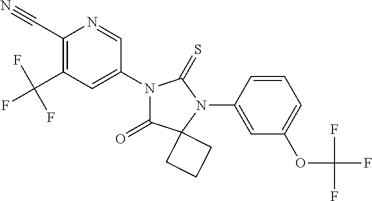 [SMILES: N#Cc1ncc(N2C(=O)C3(CCC3)N(c3cccc(OC(F)(F)F)c3)C2=S)cc1C(F)(F)F] | 97 | 5-[5-oxo-7-thioxo-8-[3- (trifluoromethoxy)phenyl]-6,8- diazaspiro[3.4]octan-6-yl]-3- (trifluoromethyl)pyridine-2-carbonitrile |
| 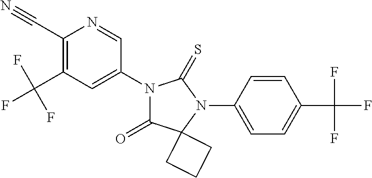 [SMILES: N#Cc1ncc(N2C(=O)C3(CCC3)N(c3ccc(C(F)(F)F)cc3)C2=S)cc1C(F)(F)F] | 98 | 5-[5-oxo-7-thioxo-8-[4- (trifluoromethyl)phenyl]-6,8- diazaspiro[3.4]octan-6-yl]-3- (trifluoromethyl)pyridine-2-carbonitrile |
| 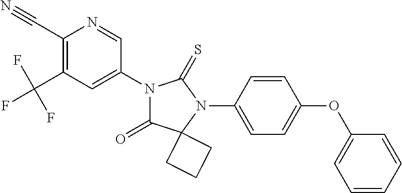 [SMILES: N#Cc1ncc(N2C(=O)C3(CCC3)N(c3ccc(Oc4ccccc4)cc3)C2=S)cc1C(F)(F)F] | 99 | 5-[5-oxo-8-(4-phenoxyphenyl)-7- thioxo-6,8-diazaspiro[3.4]octan-6-yl]- 3-(trifluoromethyl)pyridine-2- carbonitrile |
| 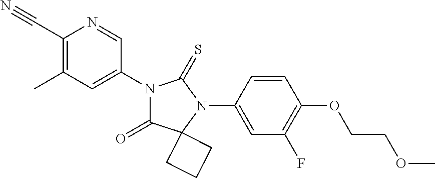 [SMILES: COCCOc1ccc(N2C(=S)N(c3cnc(C#N)c(C)c3)C(=O)C23CCC3)cc1F] | 100 | 5-[8-[3-fluoro-4-(2- methoxyethoxy)phenyl]-5-oxo-7- thioxo-6,8-diazaspiro[3.4]octan-6-yl]- 3-methyl-pyridine-2-carbonitrile |
| 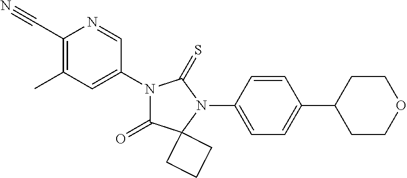 [SMILES: Cc1cc(N2C(=O)C3(CCC3)N(c3ccc(C4CCOCC4)cc3)C2=S)cnc1C#N] | 101 | 3-methyl-5-[5-oxo-8-(4- tetrahydropyran-4-ylphenyl)-7-thioxo- 6,8-diazaspiro[3.4]octan-6-yl]pyridine- 2-carbonitrile |
| 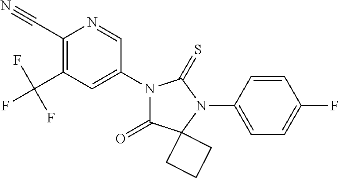 [SMILES: N#Cc1ncc(N2C(=O)C3(CCC3)N(c3ccc(F)cc3)C2=S)cc1C(F)(F)F] | 102 | 5-[8-(4-fluorophenyl)-5-oxo-7-thioxo- 6,8-diazaspiro[3.4]octan-6-yl]-3- (trifluoromethyl)pyridine-2-carbonitrile |
| 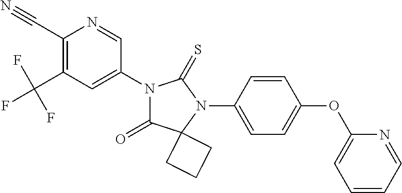 [SMILES: N#Cc1ncc(N2C(=O)C3(CCC3)N(c3ccc(Oc4ccccn4)cc3)C2=S)cc1C(F)(F)F] | 103 | 5-[5-oxo-8-[4-(2-pyridyloxy)phenyl]-7- thioxo-6,8-diazaspiro[3.4]octan-6-yl]- 3-(trifluoromethyl)pyridine-2- carbonitrile |
| 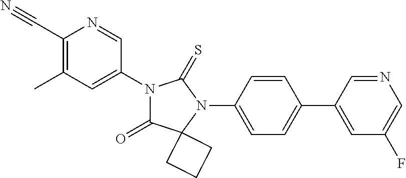 [SMILES: Cc1cc(N2C(=O)C3(CCC3)N(c3ccc(-c4cncc(F)c4)cc3)C2=S)cnc1C#N] | 104 | 5-[8-[4-(5-fluoro-3-pyridyl)phenyl]-5- oxo-7-thioxo-6,8-diazaspiro[3.4]octan- 6-yl]-3-methyl-pyridine-2-carbonitrile |
| 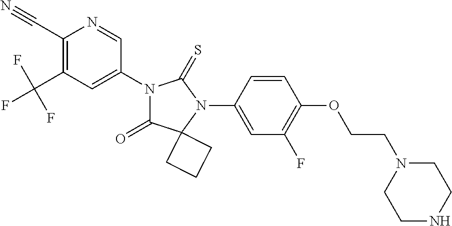 [SMILES: N#Cc1ncc(N2C(=O)C3(CCC3)N(c3ccc(OCCN4CCNCC4)c(F)c3)C2=S)cc1C(F)(F)F] | 105 | 5-[8-[3-fluoro-4-(2-piperazin-1- ylethoxy)phenyl]-5-oxo-7-thioxo-6,8- diazaspiro[3.4]octan-6-yl]-3- (trifluoromethyl)pyridine-2-carbonitrile |
| 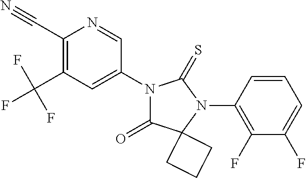 [SMILES: N#Cc1ncc(N2C(=O)C3(CCC3)N(c3cccc(F)c3F)C2=S)cc1C(F)(F)F] | 106 | 5-[8-(2,3-difluorophenyl)-5-oxo-7- thioxo-6,8-diazaspiro[3.4]octan-6-yl]- 3-(trifluoromethyl)pyridine-2- carbonitrile |
| 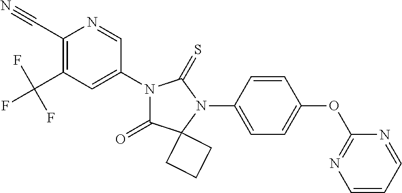 [SMILES: N#Cc1ncc(N2C(=O)C3(CCC3)N(c3ccc(Oc4ncccn4)cc3)C2=S)cc1C(F)(F)F] | 107 | 5-[5-oxo-8-(4-pyrimidin-2- yloxyphenyl)-7-thioxo-6,8- diazaspiro[3.4]octan-6-yl]-3- (trifluoromethyl)pyridine-2-carbonitrile |
| 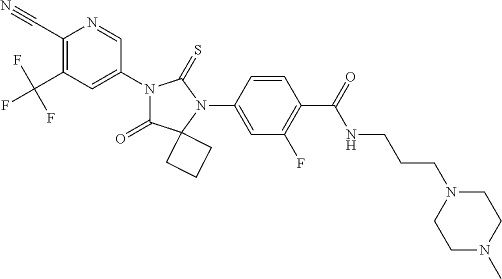 [SMILES: CN1CCN(CCCNC(=O)c2ccc(N3C(=S)N(c4cnc(C#N)c(C(F)(F)F)c4)C(=O)C34CCC4)cc2F)CC1] | 108 | 4-[6-[6-cyano-5-(trifluoromethyl)-3- pyridyl]-5-oxo-7-thioxo-6,8- diazaspiro[3.4]octan-8-yl]-2-fluoro-N- [3-(4-methylpiperazin-1- yl)propyl]benzamide |
| 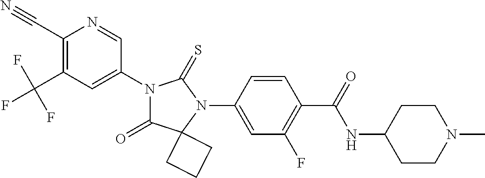 [SMILES: CN1CCC(NC(=O)c2ccc(N3C(=S)N(c4cnc(C#N)c(C(F)(F)F)c4)C(=O)C34CCC4)cc2F)CC1] | 109 | 4-[6-[6-cyano-5-(trifluoromethyl)-3- pyridyl]-5-oxo-7-thioxo-6,8- diazaspiro[3.4]octan-8-yl]-2-fluoro-N- (1-methyl-4-piperidyl)benzamide |
| 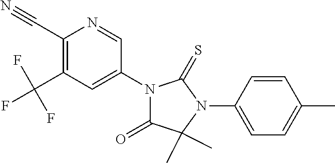 [SMILES: Cc1ccc(N2C(=S)N(c3cnc(C#N)c(C(F)(F)F)c3)C(=O)C2(C)C)cc1] | 110 | 5-[4,4-dimethyl-5-oxo-3-(p-tolyl)-2- thioxo-imidazolidin-1-yl]-3- (trifluoromethyl)pyridine-2-carbonitrile |
| 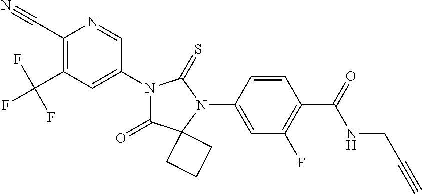 [SMILES: C#CCNC(=O)c1ccc(N2C(=S)N(c3cnc(C#N)c(C(F)(F)F)c3)C(=O)C23CCC3)cc1F] | 111 | 4-[6-[6-cyano-5-(trifluoromethyl)-3- pyridyl]-5-oxo-7-thioxo-6,8- diazaspiro[3.4]octan-8-yl]-2-fluoro-N- prop-2-ynyl-benzamide |
| 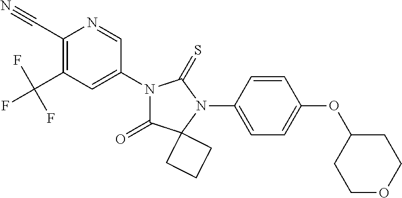 [SMILES: N#Cc1ncc(N2C(=O)C3(CCC3)N(c3ccc(OC4CCOCC4)cc3)C2=S)cc1C(F)(F)F] | 112 | 5-[5-oxo-8-(4-tetrahydropyran-4- yloxyphenyl)-7-thioxo-6,8- diazaspiro[3.4]octan-6-yl]-3- (trifluoromethyl)pyridine-2-carbonitrile |
| 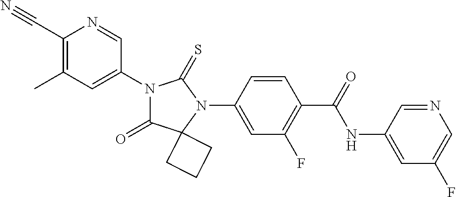 [SMILES: Cc1cc(N2C(=O)C3(CCC3)N(c3ccc(C(=O)Nc4cncc(F)c4)c(F)c3)C2=S)cnc1C#N] | 113 | 4-[6-(6-cyano-5-methyl-3-pyridyl)-5- oxo-7-thioxo-6,8-diazaspiro[3.4]octan- 8-yl]-2-fluoro-N-(5-fluoro-3- pyridyl)benzamide |
| 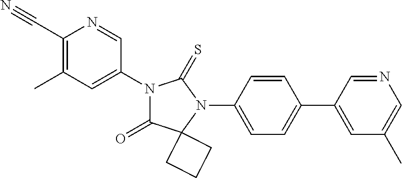 [SMILES: Cc1cncc(-c2ccc(N3C(=S)N(c4cnc(C#N)c(C)c4)C(=O)C34CCC4)cc2)c1] | 114 | 3-methyl-5-[8-[4-(5-methyl-3- pyridyl)phenyl]-5-oxo-7-thioxo-6,8- diazaspiro[3.4]octan-6-yl]pyridine-2- carbonitrile |
| 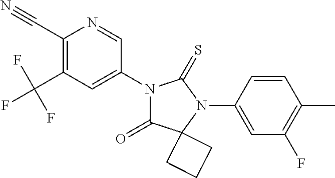 [SMILES: Cc1ccc(N2C(=S)N(c3cnc(C#N)c(C(F)(F)F)c3)C(=O)C23CCC3)cc1F] | 115 | 5-[8-(3-fluoro-4-methyl-phenyl)-5-oxo- 7-thioxo-6,8-diazaspiro[3.4]octan-6- yl]-3-(trifluoromethyl)pyridine-2- carbonitrile |
| 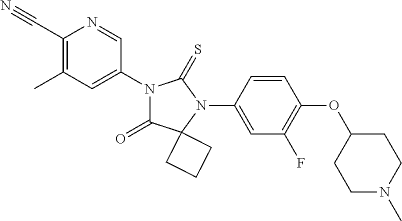 [SMILES: Cc1cc(N2C(=O)C3(CCC3)N(c3ccc(OC4CCN(C)CC4)c(F)c3)C2=S)cnc1C#N] | 116 | 5-[8-[3-fluoro-4-[(1-methyl-4- piperidyl)oxy]phenyl]-5-oxo-7-thioxo- 6,8-diazaspiro[3.4]octan-6-yl]-3- methyl-pyridine-2-carbonitrile |
| 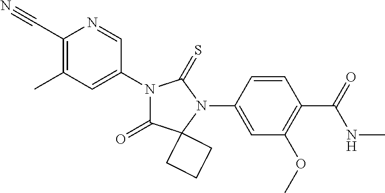 [SMILES: CNC(=O)c1ccc(N2C(=S)N(c3cnc(C#N)c(C)c3)C(=O)C23CCC3)cc1OC] | 117 | 4-[6-(6-cyano-5-methyl-3-pyridyl)-5- oxo-7-thioxo-6,8-diazaspiro[3.4]octan- 8-yl]-2-methoxy-N-methyl-benzamide |
| 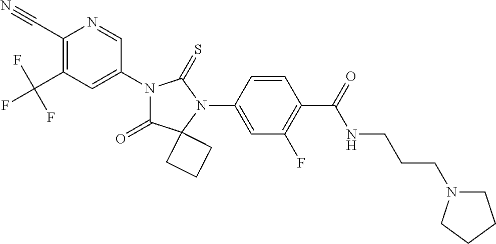 [SMILES: N#Cc1ncc(N2C(=O)C3(CCC3)N(c3ccc(C(=O)NCCCN4CCCC4)c(F)c3)C2=S)cc1C(F)(F)F] | 118 | 4-[6-[6-cyano-5-(trifluoromethyl)-3- pyridyl]-5-oxo-7-thioxo-6,8- diazaspiro[3.4]octan-8-yl]-2-fluoro-N- (3-pyrrolidin-1-ylpropyl)benzamide |
| 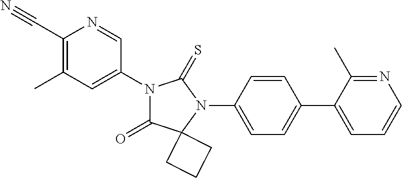 [SMILES: Cc1cc(N2C(=O)C3(CCC3)N(c3ccc(-c4cccnc4C)cc3)C2=S)cnc1C#N] | 119 | 3-methyl-5-[8-[4-(2-methyl-3- pyridyl)phenyl]-5-oxo-7-thioxo-6,8- diazaspiro[3.4]octan-6-yl]pyridine-2- carbonitrile |
| 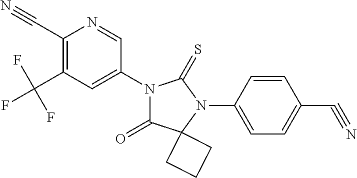 [SMILES: N#Cc1ccc(N2C(=S)N(c3cnc(C#N)c(C(F)(F)F)c3)C(=O)C23CCC3)cc1] | 120 | 5-[8-(4-cyanophenyl)-5-oxo-7-thioxo- 6,8-diazaspiro[3.4]octan-6-yl]-3- (trifluoromethyl)pyridine-2-carbonitrile |
| 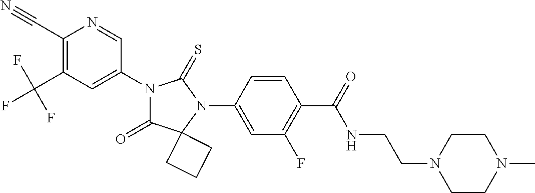 [SMILES: CN1CCN(CCNC(=O)c2ccc(N3C(=S)N(c4cnc(C#N)c(C(F)(F)F)c4)C(=O)C34CCC4)cc2F)CC1] | 121 | 4-[6-[6-cyano-5-(trifluoromethyl)-3- pyridyl]-5-oxo-7-thioxo-6,8- diazaspiro[3.4]octan-8-yl]-2-fluoro-N- [2-(4-methylpiperazin-1- yl)ethyl]benzamide |
| 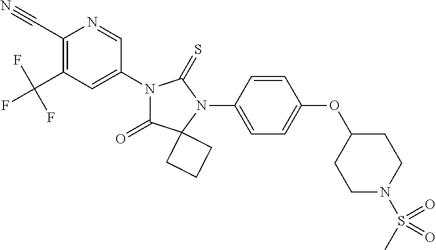 [SMILES: CS(=O)(=O)N1CCC(Oc2ccc(N3C(=S)N(c4cnc(C#N)c(C(F)(F)F)c4)C(=O)C34CCC4)cc2)CC1] | 122 | 5-[8-[4-[(1-methylsulfonyl-4- piperidyl)oxy]phenyl]-5-oxo-7-thioxo- 6,8-diazaspiro[3.4]octan-6-yl]-3- (trifluoromethyl)pyridine-2-carbonitrile |

A further embodiment of the present invention is directed to a method for treating and/or ameliorating diseases, syndromes, disorders, or conditions associated with AR mutant receptors linked to castration-resistant prostate cancer, in a subject, including a mammal and/or human, in need thereof, who has demonstrated resistance to a first or second generation AR antagonist, comprising, consisting of, and/or consisting essentially of, administering to a subject in need thereof, a therapeutically effective amount of a compound of Formula (I)

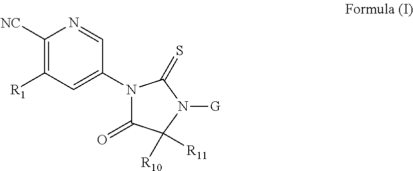

or a pharmaceutically acceptable salt form thereof,
selected from the group consisting of
Cpd 1, 5-[4,4-dimethyl-3-[4-[(1-methyl-4-piperidyl)oxy]phenyl]-5-oxo-2-thioxo-imidazolidin-1-yl]-3-methyl-pyridine-2-carbonitrile;
Cpd 2, 4-[6-(6-cyano-5-methyl-3-pyridyl)-5-oxo-7-thioxo-6,8-diazaspiro[3.4]octan-8-yl]-2-fluoro-N-tetrahydropyran-4-yl-benzamide;
Cpd 3, 4-[6-(6-cyano-5-methyl-3-pyridyl)-5-oxo-7-thioxo-6,8-diazaspiro[3.4]octan-8-yl]-2-fluoro-N-(tetrahydropyran-4-ylmethyl)benzamide;
Cpd 4, 3-methyl-5-[8-[4-[(1-methyl-4-piperidyl)oxy]phenyl]-5-oxo-7-thioxo-6,8-diazaspiro[3.4]octan-6-yl]pyridine-2-carbonitrile;
Cpd 5, 4-[6-(6-cyano-5-methyl-3-pyridyl)-5-oxo-7-thioxo-6,8-diazaspiro[3.4]octan-8-yl]-N,2-dimethyl-benzamide;
Cpd 6, 5-[8-[4-(1, 1-dioxothian-4-yl)oxyphenyl]-5-oxo-7-thioxo-6,8-diazaspiro[3.4]octan-6-yl]-3-methyl-pyridine-2-carbonitrile;
Cpd 7, 5-[8-(7-isoquinolyl)-5-oxo-7-thioxo-6,8-diazaspiro[3.4]octan-6-yl]-3-(trifluoromethyl)pyridine-2-carbonitrile;
Cpd 8, 5-[5-oxo-8-(4-piperazin-1-ylphenyl)-7-thioxo-6,8-diazaspiro[3.4]octan-6-yl]-3-(trifluoromethyl)pyridine-2-carbonitrile
Cpd 9, 5-[5-oxo-8-(3-pyridyl)-7-thioxo-6,8-diazaspiro[3.4]octan-6-yl]-3-(trifluoromethyl)pyridine-2-carbonitrile;
Cpd 10, 3-methyl-5-[8-[4-[2-(4-methylpiperazin-1-yl)ethoxy]phenyl]-5-oxo-7-thioxo-6,8-diazaspiro[3.4]octan-6-yl]pyridine-2-carbonitrile;
Cpd 11, 4-[6-(6-cyano-5-methyl-3-pyridyl)-5-oxo-7-thioxo-6,8-diazaspiro[3.4]octan-8-yl]-2-fluoro-N-(2-fluorophenyl)benzamide;
Cpd 12, 4-[3-(6-cyano-5-methyl-3-pyridyl)-5,5-dimethyl-4-oxo-2-thioxo-imidazolidin-1-yl]-2-fluoro-N-methyl-benzamide;
Cpd 13, 3-methyl-5-[5-oxo-8-(4-tetrahydrothiopyran-4-yloxyphenyl)-7-thioxo-6,8-diazaspiro[3.4]octan-6-yl]pyridine-2-carbonitrile;
Cpd 14, 4-[6-[6-cyano-5-(trifluoromethyl)-3-pyridyl]-5-oxo-7-thioxo-6,8-diazaspiro[3.4]octan-8-yl]-2-fluoro-N-[2-(4-pyridyl)ethyl]benzamide;
Cpd 15, 4-[6-[6-cyano-5-(trifluoromethyl)-3-pyridyl]-5-oxo-7-thioxo-6,8-diazaspiro[3.4]octan-8-yl]-2-fluoro-N-[2-(2-pyridyl)ethyl]benzamide;
Cpd 16, 4-[6-(6-cyano-5-methyl-3-pyridyl)-5-oxo-7-thioxo-6,8-diazaspiro[3.4]octan-8-yl]-2-fluoro-N-methyl-benzamide;
Cpd 17, 4-[6-[6-cyano-5-(trifluoromethyl)-3-pyridyl]-5-oxo-7-thioxo-6,8-diazaspiro[3.4]octan-8-yl]-2-fluoro-N-(2-hydroxy-2-methyl-propyl)benzamide;
Cpd 18, 5-[8-(2-naphthyl)-5-oxo-7-thioxo-6,8-diazaspiro[3.4]octan-6-yl]-3-(trifluoromethyl)pyridine-2-carbonitrile;
Cpd 19, 5-[4,4-dimethyl-3-[4-[(1-methyl-4-piperidyl)oxy]phenyl]-5-oxo-2-thioxo-imidazolidin-1-yl]-3-(trifluoromethyl)pyridine-2-carbonitrile;
Cpd 20, 5-[5-oxo-8-(3-phenoxyphenyl)-7-thioxo-6,8-diazaspiro[3.4]octan-6-yl]-3-(trifluoromethyl)pyridine-2-carbonitrile;
Cpd 21, 4-[6-[6-cyano-5-(trifluoromethyl)-3-pyridyl]-5-oxo-7-thioxo-6,8-diazaspiro[3.4]octan-8-yl]-N-(cyclopentylmethyl)-2-fluoro-benzamide;
Cpd 22, 4-[6-[6-cyano-5-(trifluoromethyl)-3-pyridyl]-5-oxo-7-thioxo-6,8-diazaspiro[3.4]octan-8-yl]-2-fluoro-N-(2-morpholinoethyl)benzamide;
Cpd 23, 4-[6-[6-cyano-5-(trifluoromethyl)-3-pyridyl]-5-oxo-7-thioxo-6,8-diazaspiro[3.4]octan-8-yl]-2-fluoro-N-isopropyl-benzamide;
Cpd 24, 4-[6-[6-cyano-5-(trifluoromethyl)-3-pyridyl]-5-oxo-7-thioxo-6,8-diazaspiro[3.4]octan-8-yl]-2-fluoro-N-(3-methoxypropyl)benzamide;
Cpd 25, 4-[6-[6-cyano-5-(trifluoromethyl)-3-pyridyl]-5-oxo-7-thioxo-6,8-diazaspiro[3.4]octan-8-yl]-N-methyl-benzensulfonamide;
Cpd 26, 4-[6-[6-cyano-5-(trifluoromethyl)-3-pyridyl]-5-oxo-7-thioxo-6,8-diazaspiro[3.4]octan-8-yl]-2-fluoro-N-[(5-methyl-2-furyl)methyl]benzamide;
Cpd 27, 4-[6-[6-cyano-5-(trifluoromethyl)-3-pyridyl]-5-oxo-7-thioxo-6,8-diazaspiro[3.4]octan-8-yl]-2-fluoro-N-isopentyl-benzamide;
Cpd 28, 5-[8-[3-fluoro-4-[(1-methyl-4-piperidyl)oxy]phenyl]-5-oxo-7-thioxo-6,8-diazaspiro[3.4]octan-6-yl]-3-(trifluoromethyl)pyridine-2-carbonitrile;
Cpd 29, 3-methyl-5-[5-oxo-8-(p-tolyl)-7-thioxo-6,8-diazaspiro[3.4]octan-6-yl]pyridine-2-carbonitrile;
Cpd 30, 5-[8-[4-[(4-methylpiperazin-1-yl)methyl]phenyl]-5-oxo-7-thioxo-6,8-diazaspiro[3.4]octan-6-yl]-3-(trifluoromethyl)pyridine-2-carbonitrile;
Cpd 31, N-[(2-chlorophenyl)methyl]-4-[6-[6-cyano-5-(trifluoromethyl)-3-pyridyl]-5-oxo-7-thioxo-6,8-diazaspiro[3.4]octan-8-yl]-2-fluoro-benzamide;
Cpd 32, 5-[8-[3-fluoro-4-[2-(2-pyridyl)ethoxy]phenyl]-5-oxo-7-thioxo-6,8-diazaspiro[3.4]octan-6-yl]-3-methyl-pyridine-2-carbonitrile;
Cpd 33, 4-[6-(6-cyano-5-methyl-3-pyridyl)-5-oxo-7-thioxo-6,8-diazaspiro[3.4]octan-8-yl]-2-fluoro-N-(2-thienylmethyl)benzamide;
Cpd 34, 5-[8-(1-naphthyl)-5-oxo-7-thioxo-6,8-diazaspiro[3.4]octan-6-yl]-3-(trifluoromethyl)pyridine-2-carbonitrile;
Cpd 35, 5-[5-oxo-8-[4-(4-piperidyloxy)phenyl]-7-thioxo-6,8-diazaspiro[3.4]octan-6-yl]-3-(trifluoromethyl)pyridine-2-carbonitrile;
Cpd 36, N-benzyl-4-[6-[6-cyano-5-(trifluoromethyl)-3-pyridyl]-5-oxo-7-thioxo-6,8-diazaspiro[3.4]octan-8-yl]-2-fluoro-benzamide;
Cpd 37, 4-[6-[6-cyano-5-(trifluoromethyl)-3-pyridyl]-5-oxo-7-thioxo-6,8-diazaspiro[3.4]octan-8-yl]-2-fluoro-N-[3-(2-oxopyrrolidin-1-yl)propyl]benzamide;
Cpd 38, 5-[5-oxo-7-thioxo-8-[4-(trifluoromethoxy)phenyl]-6,8-diazaspiro[3.4]octan-6-yl]-3-(trifluoromethyl)pyridine-2-carbonitrile;
Cpd 39, 4-[6-[6-cyano-5-(difluoromethyl)-3-pyridyl]-5-oxo-7-thioxo-6,8-diazaspiro[3.4]octan-8-yl]-2-fluoro-N-methyl-benzamide;
Cpd 40, 4-[6-[6-cyano-5-(trifluoromethyl)-3-pyridyl]-5-oxo-7-thioxo-6,8-diazaspiro[3.4]octan-8-yl]-2-fluoro-N-(3-hydroxypropyl)benzamide;
Cpd 41, ethyl 2-[[4-[6-[6-cyano-5-(trifluoromethyl)-3-pyridyl]-5-oxo-7-thioxo-6,8-diazaspiro[3.4]octan-8-yl]-2-fluoro-benzoyl]amino]acetate;
Cpd 42, 4-[6-[6-cyano-5-(trifluoromethyl)-3-pyridyl]-5-oxo-7-thioxo-6,8-diazaspiro[3.4]octan-8-yl]-2-fluoro-N-phenethyl-benzamide;
Cpd 43, 5-[8-[4-[3-(4-methylpiperazin-1-yl)propyl]phenyl]-5-oxo-7-thioxo-6,8-diazaspiro[3.4]octan-6-yl]-3-(trifluoromethyl)pyridine-2-carbonitrile;
Cpd 44, 4-[6-[6-cyano-5-(trifluoromethyl)-3-pyridyl]-5-oxo-7-thioxo-6,8-diazaspiro[3.4]octan-8-yl]-2-fluoro-N-(2-pyridylmethyl)benzamide;
Cpd 45, 4-[6-(6-cyano-5-methyl-3-pyridyl)-5-oxo-7-thioxo-6,8-diazaspiro[3.4]octan-8-yl]-2-fluoro-N-(2-methylpyrazol-3-yl)benzamide;
Cpd 46, 4-[6-[6-cyano-5-(trifluoromethyl)-3-pyridyl]-5-oxo-7-thioxo-6,8-diazaspiro[3.4]octan-8-yl]-2-fluoro-N-(2-methoxyethyl)benzamide;
Cpd 47, 5-[8-(2-fluoro-4-hydroxy-phenyl)-5-oxo-7-thioxo-6,8-diazaspiro[3.4]octan-6-yl]-3-(trifluoromethyl)pyridine-2-carbonitrile;
Cpd 48, 5-[8-(4-hydroxyphenyl)-5-oxo-7-thioxo-6,8-diazaspiro[3.4]octan-6-yl]-3-methyl-pyridine-2-carbonitrile;
Cpd 49, 5-[8-[2-fluoro-4-[(1-methyl-4-piperidyl)oxy]phenyl]-5-oxo-7-thioxo-6,8-diazaspiro[3.4]octan-6-yl]-3-(trifluoromethyl)pyridine-2-carbonitrile;
Cpd 50, 3-methyl-5-[8-[4-(5-methyl-2-furyl)phenyl]-5-oxo-7-thioxo-6,8-diazaspiro[3.4]octan-6-yl]pyridine-2-carbonitrile;
Cpd 51, 5-[8-[4-[[1-(2-hydroxyethyl)-4-piperidyl]oxy]phenyl]-5-oxo-7-thioxo-6,8-diazaspiro[3.4]octan-6-yl]-3-(trifluoromethyl)pyridine-2-carbonitrile;
Cpd 52, 5-[8-[3-fluoro-4-(pyrrolidine-1-carbonyl)phenyl]-5-oxo-7-thioxo-6,8-diazaspiro[3.4]octan-6-yl]-3-(trifluoromethyl)pyridine-2-carbonitrile;
Cpd 53, N-[(4-chlorophenyl)methyl]-4-[6-[6-cyano-5-(trifluoromethyl)-3-pyridyl]-5-oxo-7-thioxo-6,8-diazaspiro[3.4]octan-8-yl]-2-fluoro-benzamide;
Cpd 54, 4-[6-[6-cyano-5-(trifluoromethyl)-3-pyridyl]-5-oxo-7-thioxo-6,8-diazaspiro[3.4]octan-8-yl]-2-fluoro-N-(pyrazin-2-ylmethyl)benzamide;
Cpd 55, 5-[8-[3-fluoro-4-(3-pyrrolidin-1-ylpropoxy)phenyl]-5-oxo-7-thioxo-6,8-diazaspiro[3.4]octan-6-yl]-3-(trifluoromethyl)pyridine-2-carbonitrile;
Cpd 56, 5-[8-[3-fluoro-4-(2-pyrimidin-2-ylethoxy)phenyl]-5-oxo-7-thioxo-6,8-diazaspiro[3.4]octan-6-yl]-3-(trifluoromethyl)pyridine-2-carbonitrile;
Cpd 57, 4-[6-[6-cyano-5-(trifluoromethyl)-3-pyridyl]-5-oxo-7-thioxo-6,8-diazaspiro[3.4]octan-8-yl]-2-fluoro-N-(3-phenylpropyl)benzamide;
Cpd 58, N-butyl-4-[6-[6-cyano-5-(trifluoromethyl)-3-pyridyl]-5-oxo-7-thioxo-6,8-diazaspiro[3.4]octan-8-yl]-2-fluoro-benzamide;
Cpd 59, 4-[6-[6-cyano-5-(trifluoromethyl)-3-pyridyl]-5-oxo-7-thioxo-6,8-diazaspiro[3.4]octan-8-yl]-2-fluoro-N-(2-thienylmethyl)benzamide;
Cpd 60, 5-[8-[4-[[1-(2-hydroxyethyl)-4-piperidyl]oxy]phenyl]-5-oxo-7-thioxo-6,8-diazaspiro[3.4]octan-6-yl]-3-methyl-pyridine-2-carbonitrile;
Cpd 61, 5-[8-[4-[(1-ethyl-4-piperidyl)oxy]phenyl]-5-oxo-7-thioxo-6,8-diazaspiro[3.4]octan-6-yl]-3-methyl-pyridine-2-carbonitrile;
Cpd 62, 3-methyl-5-[5-oxo-8-(4-pyrimidin-4-yloxyphenyl)-7-thioxo-6,8-diazaspiro[3.4]octan-6-yl]pyridine-2-carbonitrile;
Cpd 63, 4-[6-(6-cyano-5-methyl-3-pyridyl)-5-oxo-7-thioxo-6,8-diazaspiro[3.4]octan-8-yl]-2-fluoro-N-thiazol-2-yl-benzamide;
Cpd 64, 4-[6-[6-cyano-5-(trifluoromethyl)-3-pyridyl]-5-oxo-7-thioxo-6,8-diazaspiro[3.4]octan-8-yl]-N-[3-[cyclopentyl(methyl)amino]propyl]-2-fluoro-benzamide;
Cpd 65, 4-[7-(6-cyano-5-methyl-3-pyridyl)-6-oxo-8-thioxo-7,9-diazaspiro[4.4]nonan-9-yl]-2-fluoro-N-methyl-benzamide;
Cpd 66, 5-[8-[3-fluoro-4-(2-pyrrolidin-1-ylethoxy)phenyl]-5-oxo-7-thioxo-6,8-diazaspiro[3.4]octan-6-yl]-3-(trifluoromethyl)pyridine-2-carbonitrile;
Cpd 67, 4-[6-[6-cyano-5-(trifluoromethyl)-3-pyridyl]-5-oxo-7-thioxo-6,8-diazaspiro[3.4]octan-8-yl]-2-fluoro-N-(3-pyridylmethyl)benzamide;
Cpd 68, 4-[6-[6-cyano-5-(trifluoromethyl)-3-pyridyl]-5-oxo-7-thioxo-6,8-diazaspiro[3.4]octan-8-yl]-2-fluoro-N-propyl-benzamide;
Cpd 69, 5-[8-(4-methoxyphenyl)-5-oxo-7-thioxo-6,8-diazaspiro[3.4]octan-6-yl]-3-methyl-pyridine-2-carbonitrile;
Cpd 70, 5-(5-oxo-8-phenyl-7-thioxo-6,8-diazaspiro[3.4]octan-6-yl)-3-(trifluoromethyl)pyridine-2-carbonitrile;
Cpd 71, 5-[5-oxo-8-(4-pyrimidin-4-yloxyphenyl)-7-thioxo-6,8-diazaspiro[3.4]octan-6-yl]-3-(trifluoromethyl)pyridine-2-carbonitrile;
Cpd 72, 4-[6-[6-cyano-5-(trifluoromethyl)-3-pyridyl]-5-oxo-7-thioxo-6,8-diazaspiro[3.4]octan-8-yl]-2-fluoro-N-(3-morpholinopropyl)benzamide;
Cpd 73, 4-[6-[6-cyano-5-(trifluoromethyl)-3-pyridyl]-5-oxo-7-thioxo-6,8-diazaspiro[3.4]octan-8-yl]-2-fluoro-N-phenyl-benzamide;
Cpd 74, N-(4-chlorophenyl)-4-[6-(6-cyano-5-methyl-3-pyridyl)-5-oxo-7-thioxo-6,8-diazaspiro[3.4]octan-8-yl]-2-fluoro-benzamide;
Cpd 75, 4-[6-(6-cyano-5-methyl-3-pyridyl)-5-oxo-7-thioxo-6,8-diazaspiro[3.4]octan-8-yl]-2-fluoro-N-(6-methyl-3-pyridyl)benzamide;
Cpd 76, 4-[6-[6-cyano-5-(trifluoromethyl)-3-pyridyl]-5-oxo-7-thioxo-6,8-diazaspiro[3.4]octan-8-yl]-2-fluoro-N-(2-furylmethyl)benzamide;
Cpd 77, 5-[8-(4-hydroxyphenyl)-5-oxo-7-thioxo-6,8-diazaspiro[3.4]octan-6-yl]-3-(trifluoromethyl)pyridine-2-carbonitrile;
Cpd 78, N-(3-chlorophenyl)-4-[6-(6-cyano-5-methyl-3-pyridyl)-5-oxo-7-thioxo-6,8-diazaspiro[3.4]octan-8-yl]-2-fluoro-benzamide;
Cpd 79, 5-[8-(3-cyanophenyl)-5-oxo-7-thioxo-6,8-diazaspiro[3.4]octan-6-yl]-3-(trifluoromethyl)pyridine-2-carbonitrile;
Cpd 80, 4-[6-[6-cyano-5-(trifluoromethyl)-3-pyridyl]-5-oxo-7-thioxo-6,8-diazaspiro[3.4]octan-8-yl]-2-fluoro-benzamide;
Cpd 81, 5-[8-[3-(hydroxymethyl)phenyl]-5-oxo-7-thioxo-6,8-diazaspiro[3.4]octan-6-yl]-3-(trifluoromethyl)pyridine-2-carbonitrile;
Cpd 82, ethyl 4-[[4-[6-[6-cyano-5-(trifluoromethyl)-3-pyridyl]-5-oxo-7-thioxo-6,8-diazaspiro[3.4]octan-8-yl]-2-fluoro-benzoyflamino]butanoate;
Cpd 83, 3-methyl-5-[5-oxo-8-[4-(4-piperidyloxy)phenyl]-7-thioxo-6,8-diazaspiro[3.4]octan-6-yl]pyridine-2-carbonitrile;
Cpd 84, 4-[6-(6-cyano-5-methyl-3-pyridyl)-5-oxo-7-thioxo-6,8-diazaspiro[3.4]octan-8-yl]-2-fluoro-N-(4-fluorophenyl)benzamide;
Cpd 85, 5-[8-[4-[(1-methyl-4-piperidyl)oxy]phenyl]-5-oxo-7-thioxo-6,8-diazaspiro[3.4]octan-6-yl]-3-(trifluoromethyl)pyridine-2-carbonitrile;
Cpd 86, 5-[8-[4-(2-furyl)phenyl]-5-oxo-7-thioxo-6,8-diazaspiro[3.4]octan-6-yl]-3-methyl-pyridine-2-carbonitrile;
Cpd 87, 3-methyl-5-[5-oxo-8-(4-tetrahydropyran-4-yloxyphenyl)-7-thioxo-6,8-diazaspiro[3.4]octan-6-yl]pyridine-2-carbonitrile;
Cpd 88, 5-[5-oxo-8-(4-pyrimidin-5-yloxyphenyl)-7-thioxo-6,8-diazaspiro[3.4]octan-6-yl]-3-(trifluoromethyl)pyridine-2-carbonitrile;
Cpd 89, 5-[5-oxo-8-(4-tetrahydropyran-4-ylphenyl)-7-thioxo-6,8-diazaspiro[3.4]octan-6-yl]-3-(trifluoromethyl)pyridine-2-carbonitrile;
Cpd 90, 5-[8-(3-fluorophenyl)-5-oxo-7-thioxo-6,8-diazaspiro[3.4]octan-6-yl]-3-(trifluoromethyl)pyridine-2-carbonitrile;
Cpd 91, 5-[8-[2-fluoro-4-[2-(1-piperidyl)ethoxy]phenyl]-5-oxo-7-thioxo-6,8-diazaspiro[3.4]octan-6-yl]-3-(trifluoromethyl)pyridine-2-carbonitrile;
Cpd 92, 5-[8-(1H-indazol-5-yl)-5-oxo-7-thioxo-6,8-diazaspiro[3.4]octan-6-yl]-3-(trifluoromethyl)pyridine-2-carbonitrile;
Cpd 93, 3-methyl-5-[5-oxo-8-(4-pyrimidin-5-ylphenyl)-7-thioxo-6,8-diazaspiro[3.4]octan-6-yl]pyridine-2-carbonitrile;
Cpd 94, 5-[8-(4-fluoro-2-methoxy-phenyl)-5-oxo-7-thioxo-6,8-diazaspiro[3.4]octan-6-yl]-3-(trifluoromethyl)pyridine-2-carbonitrile;
Cpd 95, N-[(3-chlorophenyl)methyl]-4-[6-[6-cyano-5-(trifluoromethyl)-3-pyridyl]-5-oxo-7-thioxo-6,8-diazaspiro[3.4]octan-8-yl]-2-fluoro-benzamide;
Cpd 96, 4-[6-[6-cyano-5-(trifluoromethyl)-3-pyridyl]-5-oxo-7-thioxo-6,8-diazaspiro[3.4]octan-8-yl]-2-fluoro-N-[2-(3-pyridyl)ethyl]benzamide;
Cpd 97, 5-[5-oxo-7-thioxo-8-[3-(trifluoromethoxy)phenyl]-6,8-diazaspiro[3.4]octan-6-yl]-3-(trifluoromethyl)pyridine-2-carbonitrile;
Cpd 98, 5-[5-oxo-7-thioxo-8-[4-(trifluoromethyl)phenyl]-6,8-diazaspiro[3.4]octan-6-yl]-3-(trifluoromethyl)pyridine-2-carbonitrile;
Cpd 99, 5-[5-oxo-8-(4-phenoxyphenyl)-7-thioxo-6,8-diazaspiro[3.4]octan-6-yl]-3-(trifluoromethyl)pyridine-2-carbonitrile;
Cpd 100, 5-[8-[3-fluoro-4-(2-methoxyethoxy)phenyl]-5-oxo-7-thioxo-6,8-diazaspiro[3.4]octan-6-yl]-3-methyl-pyridine-2-carbonitrile;
Cpd 101, 3-methyl-5-[5-oxo-8-(4-tetrahydropyran-4-ylphenyl)-7-thioxo-6,8-diazaspiro[3.4]octan-6-yl]pyridine-2-carbonitrile;
Cpd 102, 5-[8-(4-fluorophenyl)-5-oxo-7-thioxo-6,8-diazaspiro[3.4]octan-6-yl]-3-(trifluoromethyl)pyridine-2-carbonitrile;
Cpd 103, 5-[5-oxo-8-[4-(2-pyridyloxy)phenyl]-7-thioxo-6,8-diazaspiro[3.4]octan-6-yl]-3-(trifluoromethyl)pyridine-2-carbonitrile;
Cpd 104, 5-[8-[4-(5-fluoro-3-pyridyl)phenyl]-5-oxo-7-thioxo-6,8-diazaspiro[3.4]octan-6-yl]-3-methyl-pyridine-2-carbonitrile;
Cpd 105, 5-[8-[3-fluoro-4-(2-piperazin-1-ylethoxy)phenyl]-5-oxo-7-thioxo-6,8-diazaspiro[3.4]octan-6-yl]-3-(trifluoromethyl)pyridine-2-carbonitrile;
Cpd 106, 5-[8-(2,3-difluorophenyl)-5-oxo-7-thioxo-6,8-diazaspiro[3.4]octan-6-yl]-3-(trifluoromethyl)pyridine-2-carbonitrile;
Cpd 107, 5-[5-oxo-8-(4-pyrimidin-2-yloxyphenyl)-7-thioxo-6,8-diazaspiro[3.4]octan-6-yl]-3-(trifluoromethyl)pyridine-2-carbonitrile;
Cpd 108, 4-[6-[6-cyano-5-(trifluoromethyl)-3-pyridyl]-5-oxo-7-thioxo-6,8-diazaspiro[3.4]octan-8-yl]-2-fluoro-N-[3-(4-methylpiperazin-1-yl)propyl]benzamide;
Cpd 109, 4-[6-[6-cyano-5-(trifluoromethyl)-3-pyridyl]-5-oxo-7-thioxo-6,8-diazaspiro[3.4]octan-8-yl]-2-fluoro-N-(1-methyl-4-piperidyl)benzamide;
Cpd 110, 5-[4,4-dimethyl-5-oxo-3-(p-tolyl)-2-thioxo-imidazolidin-1-yl]-3-(trifluoromethyl)pyridine-2-carbonitrile;
Cpd 111, 4-[6-[6-cyano-5-(trifluoromethyl)-3-pyridyl]-5-oxo-7-thioxo-6,8-diazaspiro[3.4]octan-8-yl]-2-fluoro-N-prop-2-ynyl-benzamide;
Cpd 112, 5-[5-oxo-8-(4-tetrahydropyran-4-yloxyphenyl)-7-thioxo-6,8-diazaspiro[3.4]octan-6-yl]-3-(trifluoromethyl)pyridine-2-carbonitrile;
Cpd 113, 4-[6-(6-cyano-5-methyl-3-pyridyl)-5-oxo-7-thioxo-6,8-diazaspiro[3.4]octan-8-yl]-2-fluoro-N-(5-fluoro-3-pyridyl)benzamide;
Cpd 114, 3-methyl-5-[8-[4-(5-methyl-3-pyridyl)phenyl]-5-oxo-7-thioxo-6,8-diazaspiro[3.4]octan-6-yl]pyridine-2-carbonitrile;
Cpd 115, 5-[8-[3-fluoro-4-methyl-phenyl)-5-oxo-7-thioxo-6,8-diazaspiro[3.4]octan-6-yl]-3-(trifluoromethyl)pyridine-2-carbonitrile;
Cpd 116, 5-[8-[3-fluoro-4-[(1-methyl-4-piperidyl)oxy]phenyl]-5-oxo-7-thioxo-6,8-diazaspiro[3.4]octan-6-yl]-3-methyl-pyridine-2-carbonitrile;
Cpd 117, 4-[6-(6-cyano-5-methyl-3-pyridyl)-5-oxo-7-thioxo-6,8-diazaspiro[3.4]octan-8-yl]-2-methoxy-N-methyl-benzamide;
Cpd 118, 4-[6-[6-cyano-5-(trifluoromethyl)-3-pyridyl]-5-oxo-7-thioxo-6,8-diazaspiro[3.4]octan-8-yl]-2-fluoro-N-(3-pyrrolidin-1-ylpropyl)benzamide;
Cpd 119, 3-methyl-5-[8-[4-(2-methyl-3-pyridyl)phenyl]-5-oxo-7-thioxo-6,8-diazaspiro[3.4]octan-6-yl]pyridine-2-carbonitrile;
Cpd 120, 5-[8-(4-cyanophenyl)-5-oxo-7-thioxo-6,8-diazaspiro[3.4]octan-6-yl]-3-(trifluoromethyl)pyridine-2-carbonitrile;
Cpd 121, 4-[6-[6-cyano-5-(trifluoromethyl)-3-pyridyl]-5-oxo-7-thioxo-6,8-diazaspiro[3.4]octan-8-yl]-2-fluoro-N-[2-(4-methylpiperazin-1-yl)ethyl]benzamide; and
Cpd 122, 5-[8-[4-[(1-methylsulfonyl-4-piperidyl)oxy]phenyl]-5-oxo-7-thioxo-6, 8-diazaspiro[3.4]octan-6-yl]-3-(trifluoromethyl)pyridine-2-carbonitrile.

For use in medicine, salts of compounds of Formula (I) refer to non-toxic “pharmaceutically acceptable salts.” Other salts may, however, be useful in the preparation of compounds of Formula (I) or of their pharmaceutically acceptable salt forms thereof. Suitable pharmaceutically acceptable salts of compounds of Formula (I) include acid addition salts that can, for example, be formed by mixing a solution of the compound with a solution of a pharmaceutically acceptable acid such as, hydrochloric acid, sulfuric acid, fumaric acid, maleic acid, succinic acid, acetic acid, benzoic acid, citric acid, tartaric acid, carbonic acid or phosphoric acid. Furthermore, where the compounds of Formula (I) carry an acidic moiety, suitable pharmaceutically acceptable salts thereof may include alkali metal salts such as, sodium or potassium salts; alkaline earth metal salts such as, calcium or magnesium salts; and salts formed with suitable organic ligands such as, quaternary ammonium salts. Thus, representative pharmaceutically acceptable salts include acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide, and valerate.

Representative acids and bases that may be used in the preparation of pharmaceutically acceptable salts include acids including acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, (+)-(1S)-camphor-10-sulfonic acid, capric acid, caproic acid, caprylic acid, cinnamic acid, citric acid, cyclamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucoronic acid, L-glutamic acid, α-oxo-glutaric acid, glycolic acid, hippuric acid, hydrobromic acid, hydrochloric acid, (+)-L-lactic acid, (±)-DL-lactic acid, lactobionic acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, L-pyroglutamic acid, salicylic acid, 4-amino-salicylic acid, sebaic acid, stearic acid, succinic acid, sulfuric acid, tannic acid, (+)-L-tartaric acid, thiocyanic acid, p-toluenesulfonic acid and undecylenic acid; and bases including ammonia, L-arginine, benethamine, benzathine, calcium hydroxide, choline, deanol, diethanolamine, diethylamine, 2-(diethylamino)-ethanol, ethanolamine, ethylenediamine, N-methylglucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, sodium hydroxide, triethanolamine, tromethamine, and zinc hydroxide.

Embodiments of the present invention include prodrugs of compounds of Formula (I). In general, such prodrugs will be functional derivatives of the compounds that are readily convertible in vivo into the required compound. Thus, in the methods of treating or preventing embodiments of the present invention, the term “administering” encompasses the treatment or prevention of the various diseases, conditions, syndromes and disorders described with the compound specifically disclosed or with a compound that may not be specifically disclosed, but which converts to the specified compound in vivo after administration to a patient. Conventional procedures for the selection and preparation of suitable prodrug derivatives are described, for example, in “Design of Prodrugs”, ed. H. Bundgaard, Elsevier, 1985.

Where the compounds according to embodiments of this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention. Furthermore, some of the crystalline forms for the compounds may exist as polymorphs and as such are intended to be included in the present invention. In addition, some of the compounds may form solvates with water (i.e., hydrates) or common organic solvents, and such solvates are also intended to be encompassed within the scope of this invention. The skilled artisan will understand that the term compound as used herein, is meant to include solvated compounds of Formula (I).

Where the processes for the preparation of the compounds according to certain embodiments of the invention give rise to mixture of stereoisomers, these isomers may be separated by conventional techniques such as, preparative chromatography. The compounds may be prepared in racemic form, or individual enantiomers may be prepared either by enantiospecific synthesis or by resolution. The compounds may, for example, be resolved into their component enantiomers by standard techniques such as, the formation of diastereomeric pairs by salt formation with an optically active acid such as, (−)-di-p-toluoyl-d-tartaric acid and/or (+)-di-p-toluoyl-1-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column.

One embodiment of the present invention is directed to a composition, including a pharmaceutical composition, comprising, consisting of, and/or consisting essentially of the (+)-enantiomer of a compound of Formula (I) wherein said composition is substantially free from the (−)-isomer of said compound. In the present context, substantially free means less than about 25%, preferably less than about 10%, more preferably less than about 5%, even more preferably less than about 2% and even more preferably less than about 1% of the (−)-isomer calculated as

$$\% ( + ) - enantiomer = \frac{( mass ( + ) - enantiomer )}{\begin{matrix}( mass ( + ) - enantiomer ) + \\ ( mass ( - ) - enantiomer )\end{matrix}} \times 100.$$

Another embodiment of the present invention is a composition, including a pharmaceutical composition, comprising, consisting of, and consisting essentially of the (−)-enantiomer of a compound of Formula (I) wherein said composition is substantially free from the (+)-isomer of said compound. In the present context, substantially free from means less than about 25%, preferably less than about 10%, more preferably less than about 5%, even more preferably less than about 2% and even more preferably less than about 1% of the (+)-isomer calculated as

$$\% ( - ) - enantiomer = \frac{( mass ( - ) - enantiomer )}{\begin{matrix}( mass ( + ) - enantiomer ) + \\ ( mass ( - ) - enantiomer )\end{matrix}} \times 100.$$

During any of the processes for preparation of the compounds of the various embodiments of the present invention, it may be necessary and/or desirable to protect sensitive or reactive groups on any of the molecules concerned. This may be achieved by means of conventional protecting groups such as those described in Protective Groups in Organic Chemistry, Second Edition, J. F. W. McOmie, Plenum Press, 1973; T. W. Greene & P. G. M. Wuts, Protective Groups in Organic Synthesis, John Wiley & Sons, 1991; and T. W. Greene & P. G. M. Wuts, Protective Groups in Organic Synthesis, Third Edition, John Wiley & Sons, 1999. The protecting groups may be removed at a convenient subsequent stage using methods known from the art.

Even though the compounds of embodiments of the present invention (including their pharmaceutically acceptable salts and pharmaceutically acceptable solvates) can be administered alone, they will generally be administered in admixture with a pharmaceutically acceptable carrier, a pharmaceutically acceptable excipient and/or a pharmaceutically acceptable diluent selected with regard to the intended route of administration and standard pharmaceutical or veterinary practice. Thus, particular embodiments of the present invention are directed to pharmaceutical and veterinary compositions comprising compounds of Formula (I) and at least one pharmaceutically acceptable carrier, pharmaceutically acceptable excipient, and/or pharmaceutically acceptable diluent.

By way of example, in the pharmaceutical compositions of embodiments of the present invention, the compounds of Formula (I) may be admixed with any suitable binder(s), lubricant(s), suspending agent(s), coating agent(s), solubilizing agent(s), and combinations thereof.

Solid oral dosage forms such as, tablets or capsules, containing the compounds of the present invention may be administered in at least one dosage form at a time, as appropriate. It is also possible to administer the compounds in sustained release formulations.

Additional oral forms in which the present inventive compounds may be administered include elixirs, solutions, syrups, and suspensions; each optionally containing flavoring agents and coloring agents.

Alternatively, compounds of Formula (I) can be administered by inhalation (intratracheal or intranasal) or in the form of a suppository or pessary, or they may be applied topically in the form of a lotion, solution, cream, ointment or dusting powder. For example, they can be incorporated into a cream comprising, consisting of, and/or consisting essentially of an aqueous emulsion of polyethylene glycols or liquid paraffin. They can also be incorporated, at a concentration of between about 1% and about 10% by weight of the cream, into an ointment comprising, consisting of, and/or consisting essentially of a wax or soft paraffin base together with any stabilizers and preservatives as may be required. An alternative means of administration includes transdermal administration by using a skin or transdermal patch.

The pharmaceutical compositions of the present invention (as well as the compounds of the present invention alone) can also be injected parenterally, for example, intracavernosally, intravenously, intramuscularly, subcutaneously, intradermally, or intrathecally. In this case, the compositions will also include at least one of a suitable carrier, a suitable excipient, and a suitable diluent.

For parenteral administration, the pharmaceutical compositions of the present invention are best used in the form of a sterile aqueous solution that may contain other substances, for example, enough salts and monosaccharides to make the solution isotonic with blood.

For buccal or sublingual administration, the pharmaceutical compositions of the present invention may be administered in the form of tablets or lozenges, which can be formulated in a conventional manner.

By way of further example, pharmaceutical compositions containing at least one of the compounds of Formula (I) as the active ingredient can be prepared by mixing the compound(s) with a pharmaceutically acceptable carrier, a pharmaceutically acceptable diluent, and/or a pharmaceutically acceptable excipient according to conventional pharmaceutical compounding techniques. The carrier, excipient, and diluent may take a wide variety of forms depending upon the desired route of administration (e.g., oral, parenteral, etc.). Thus, for liquid oral preparations such as, suspensions, syrups, elixirs and solutions, suitable carriers, excipients and diluents include water, glycols, oils, alcohols, flavoring agents, preservatives, stabilizers, coloring agents and the like; for solid oral preparations such as, powders, capsules, and tablets, suitable carriers, excipients and diluents include starches, sugars, diluents, granulating agents, lubricants, binders, disintegrating agents and the like. Solid oral preparations also may be optionally coated with substances such as, sugars, or be enterically coated so as to modulate the major site of absorption and disintegration. For parenteral administration, the carrier, excipient and diluent will usually include sterile water, and other ingredients may be added to increase solubility and preservation of the composition. Injectable suspensions or solutions may also be prepared utilizing aqueous carriers along with appropriate additives such as, solubilizers and preservatives.

A therapeutically effective amount of a compound of Formula (I) or a pharmaceutical composition thereof includes a dose range from about 0.1 mg to about 3000 mg, or any particular amount or range therein, in particular from about 1 mg to about 1000 mg, or any particular amount or range therein, or, more particularly, from about 10 mg to about 500 mg, or any particular amount or range therein, of active ingredient in a regimen of about 1 to about 4 times per day for an average (70 kg) human; although, it is apparent to one skilled in the art that the therapeutically effective amount for a compound of Formula (I) will vary as will the diseases, syndromes, conditions, and disorders being treated.

For oral administration, a pharmaceutical composition is preferably provided in the form of tablets containing about 1.0, about 10, about 50, about 100, about 150, about 200, about 250, and about 500 milligrams of a compound of Formula (I).

Advantageously, a compound of Formula (I) may be administered in a single daily dose, or the total daily dosage may be administered in divided doses of two, three and four times daily.

Optimal dosages of a compound of Formula (I) to be administered may be readily determined and will vary with the particular compound used, the mode of administration, the strength of the preparation, and the advancement of the disease, syndrome, condition or disorder. In addition, factors associated with the particular subject being treated, including subject gender, age, weight, diet and time of administration, will result in the need to adjust the dose to achieve an appropriate therapeutic level and desired therapeutic effect. The above dosages are thus exemplary of the average case. There can be, of course, individual instances wherein higher or lower dosage ranges are merited, and such are within the scope of this invention.

Compounds of Formula (I) may be administered in any of the foregoing compositions and dosage regimens or by means of those compositions and dosage regimens established in the art whenever use of a compound of Formula (I) is required for a subject in need thereof.

In another embodiment of the present invention, the compounds and compositions, according to the method of the present invention, may be administered using any amount and any route of administration effective for treating a cancer or another proliferative disease, disorder or condition. In some embodiments, the cancer or other proliferative disease, disorder or condition is a prostate cancer.

In some embodiments, the cancer or other proliferative disease, disorder or condition is a castration-resistant prostate cancer (CRPC). In some embodiments, the cancer or other proliferative disease, disorder or condition is a castration-resistant prostate cancer (CRPC) bearing a mutation in AR. In some embodiments, the mutation in AR is a mutation of Phenylalanine (Phe)876.

In some embodiments, the mutation in AR is a mutation of Phe876 to leucine. In some embodiments, the mutation in AR is a mutation of Phe876 to isoleucine. In some embodiments, the mutation in AR is a mutation of Phe876 to valine. In some embodiments, the mutation in AR is a mutation of Phe876 to serine. In some embodiments, the mutation in AR is a mutation of Phe876 to cysteine. In some embodiments, the mutation in AR is a mutation of Phe876 to tyrosine.

In some embodiments, the cancer or other proliferative disease, disorder or condition is a prostate cancer that is resistant to any AR therapy as a consequence of mutation.

In some embodiments, the cancer or other proliferative disease, disorder or condition is a prostate cancer that is resistant to treatment using second-generation AR antagonists, including, but not limited to, Enzalutamide or ARN-509.

The present invention encompasses the recognition that mutations in the AR polypeptide can render the AR polypeptide resistant to anti-androgens or convert anti-androgens to androgen agonists. In some embodiments, the present invention provides compounds that can be used to effect anti-androgenic effects despite the presence of such mutations.

The amino acid sequence of an AR polypeptide described herein can exist in a mutant AR containing, or can be modified to produce an mutant AR polypeptide variant at least one (e.g., 1, 2, 3, 4, 5, 6, 7, 8, 9, 10, or more) additions, substitutions, or deletions of a wild-type amino acid residue.

In some embodiments, the AR polypeptide variants described herein result in a loss of inhibition of AR activity by one or more antiandrogens of 0, 1, 2, 3, 4, 5, 6, 7, 8, 9, 10 up to 100%. In some embodiments, the AR polypeptide variants described herein convert antiandrogens to androgen receptor agonists.

Specific, nonlimiting amino acid residues that can be modified in an AR mutant include, e.g., E566, E589, E669, C687, A700, N772, H777, C785, F877, K911, of the AR polypeptide. These amino acid residues can be substituted with any amino acid or amino acid analog. For example, the substitutions at the recited positions can be made with any of the naturally-occurring amino acids (e.g., alanine, aspartic acid, asparagine, arginine, cysteine, glycine, glutamic acid, glutamine, histidine, leucine, valine, isoleucine, lysine, methionine, proline, threonine, serine, phenylalanine, tryptophan, or tyrosine). In particular instances, an amino acid substitution is E566K, E589K, E669K, C687Y, A700T, N772S, H777Y, C785R, F877C, F877I, F877L, F877S, F877V, F877Y and/or K911E.

In some embodiments, the AR mutants as described herein can include additional modifications of the AR polypeptide previously described in the art, including but not limited to, e.g., A597T, S648G, P683T, D696E, R727H, N728I, I738F, W741L, W741C, W741L, M743V, G751S, A871V, H874Y, T878A, T878S, and P914S.

In some embodiments, the compounds and compositions, according to the method of the present invention, may be administered using any amount and any route of administration effective for treating a bone disease, disorder or condition. In some embodiments, the bone disease, disorder or condition is osteoporosis.

The present invention is directed to the use of a compound of Formula (I) for the treatment of a disease, a syndrome, a condition or a disorder in a subject, including an animal, a mammal and a human in which the disease, the syndrome, the condition or the disorder is affected by the antagonism of the androgen receptor and who has demonstrated resistance to a first or second generation AR antagonist, selected from the group consisting of prostate cancer, castration-resistant prostate cancer, and metastatic castration-resistant prostate cancer.

In certain embodiments, a compound of Formula (I), or a composition thereof, may be administered in combination with another modulator, agonist or antagonist of AR. In some embodiments, the compound of Formula (I), or composition thereof, may be administered in combination with one or more other therapeutic agents.

In some embodiments the AR modulators, agonists or antagonists include, but are not limited to gonadotropin-releasing hormone agonists or antagonists (e.g. Lupron, Zoladex (Goserelin), Degarelix, Ozarelix, ABT-620 (Elagolix), TAK-385 (Relugolix), EP-100 or KLH-2109); non-steroidal antiandrogens, aminoglutethimide, enzalutamide, bicalutamide, nilutamide, flutamide, steroidal antiandrogens, finasteride, dutasteride, bexlosteride, izonsteride, turosteride, epristeride, other inhibitors of 5-alphareductase, 3,3′-diindolylmethane (DIM), N-butylbenzene-sulfonamide (NBBS); or a CYP17 inhibitor such as abiraterone acetate, TAK-700 (orteronel), TOK-001 (galeterone) or VT-464.

A further embodiment of the present invention is directed to the use of a pharmaceutical composition comprising, consisting of, and/or consisting essentially of a compound of Formula (I) and abiraterone acetate, for treating and/or ameliorating diseases, syndromes, disorders, or conditions associated with AR mutant receptors linked to castration-resistant prostate cancer, in a subject, including a mammal and/or human, in need thereof, who has demonstrated resistance to a first or second generation AR antagonist, comprising, consisting of, and/or consisting essentially of, administering to the subject in need thereof, a therapeutically effective amount of said pharmaceutical composition.

A further embodiment of the present invention is directed to the use of a pharmaceutical composition comprising, consisting of, and/or consisting essentially of a compound of Formula (I) and abiraterone acetate and, optionally, prednisone or dexamethasone, for treating and/or ameliorating diseases, syndromes, disorders, or conditions associated with AR mutant receptors linked to castration-resistant prostate cancer, in a subject, including a mammal and/or human, in need thereof, who has demonstrated resistance to a first or second generation AR antagonist comprising, consisting of, and/or consisting essentially of, administering to the subject in need thereof, a therapeutically effective amount of said pharmaceutical composition.

In certain embodiments, a compound of Formula (I), or a pharmaceutical composition thereof, may be administered in combination with a PI3K pathway inhibitor.

In some embodiments the PI3K pathway inhibitors (PI3K, TORC or dual PI3K/TORC inhibitor) include, but are not limited to, everolimus, BEZ-235, BKM120, BGT226, BYL-719, GDC0068, GDC-0980, GDC0941, GDC0032, MK-2206, OSI-027, CC-223, AZD8055, SAR245408, SAR245409, PF04691502, WYE125132, GSK2126458, GSK-2636771, BAY806946, PF-05212384, SF1126, PX866, AMG319, ZSTK474, CallOl, PWT33597, LY-317615 (enzastaurin hydrochloride), CU-906, or CUDC-907.

In certain embodiments, a compound of Formula (I), or a composition thereof, may be administered in combination with radiation therapy. The term “radiotherapy” or “ionizing radiation” include all forms of radiation, including but not limited to α, β, and γ radiation and ultraviolet light.

In some embodiments radiation therapy includes, but is not limited to, radioactive implants directly inserted in a tumor or body cavity (brachytherapy, interstitial irradiation, and intracavitary irradiation are types of internal radiotherapy), radiopharmaceuticals (e.g. Alpharadin (Radium-223 Chloride), 177Lu-J591 PSMA conjugate), or external beam radiation therapy (including Proton beam).

In certain embodiments, a compound of Formula (I), or a pharmaceutical composition thereof, may be administered in combination with immunotherapy.

In some embodiments the immunotherapy includes, but is not limited to Provenge, Prostvac, Ipilimumab, a CTLA-4 inhibitor or a PD-1 inhibitor.

### Synthetic Methods and Specific Examples

The preparation of the compounds of the present invention may be found in the U.S. patent application entitled, Androgen Receptor Modulators and Uses Thereof, U.S. NonProvisional application Ser. No. 13/579,009, filed on Feb. 16, 2011, which claims the benefit of U.S. provisional patent application No. 61/305,082, filed on Feb. 16, 2010, is hereby incorporated by reference.

### Biological Examples

The term “biological sample”, as used herein, includes, without limitation, cell cultures or extracts thereof biopsied material obtained from a mammal or extracts thereof; and blood, saliva, urine, feces, semen, tears, or other body fluids or extracts thereof.

Antagonism of receptors in a biological sample is useful for a variety of purposes that are known to one of skill in the art. Examples of such purposes include, but are not limited to, biological assays, gene expression studies, and biological target identification.

Certain embodiments of the present invention are directed to a method of treatment by antagonizing AR in a patient or a subject in need of such treatment, and who has demonstrated resistance to a first or second generation AR antagonist, comprising the step of administering to said patient a compound of Formula (I) of the present invention, or a composition comprising said compound.

The activity of a compound of Formula (I) as an antagonist of AR or for the treatment of an AR-mediated disease, disorder or condition, may be assayed in vitro or in vivo. An in vivo assessment of the efficacy of the compounds of the invention may be made using an animal model of an AR-mediated disease, disorder or condition, e.g., a rodent or primate model. The in vivo assessment may be further defined as an androgen dependent organ development (Hershberger) assay or as a tumor xenograft model. Cell-based assays may be performed using, e.g., a cell line isolated from a tissue that expresses either wild type or mutant AR. Additionally, biochemical or mechanism based assays, e.g., transcription assays using a purified protein, Northern blot, RT-PCR, etc., may be performed.

In vitro assays include assays that determine cell morphology, protein expression, and/or the cytotoxicity, enzyme inhibitory activity, and/or the subsequent functional consequences of treatment of cells with compounds of the invention. Alternate or additional in vitro assays may be used to quantitate the ability of the inhibitor to bind to protein or nucleic acid molecules within the cell.

Inhibitor binding may be measured by radiolabelling the inhibitor prior to binding, isolating the inhibitor/target molecule complex and determining the amount of radiolabel bound. Alternatively or additionally, inhibitor binding may be determined by running a competition experiment where new inhibitors are incubated with purified proteins or nucleic acids bound to known radioligands. Detailed conditions of exemplary systems for assaying a compound of Formula (I) of the present invention as an antagonist of AR are set forth in the Biological Examples below.

Such assays are exemplary and not intended to limit the scope of the invention. The skilled practitioner can appreciate that modifications can be made to conventional assays to develop equivalent or other assays that can be employed to comparably assess activity or otherwise characterize compounds and/or compositions as described herein.

### In Vitro Assays

### Biological Example 1

### Radioligand Binding of Compounds to AR, GR and ER

Radioligand binding assays are performed with the cell extracts and ligands as detailed below. Complete methodology is contained within the cited publications. K_{d} values are determined by Non-Specific Incubation Detection Method.

Receptors

GR (human) (agonist radioligand) IM-9 cells (cytosol)
[³H]dexamethasone 1.5 nM 1.5 nM triamcinolone (10 μM) 6 h 4° C. Scintillation counting (Clark, A. F et al. (1996) Invest. Ophtalmol. Vis. Sci., 37: 805-813).
ER (nonselective) (human) (agonist radioligand) MCF-7 cells (cytosol)
[³H]estradiol 0.4 nM 0.2 nM 17β-estradiol (6 μM) 20 h 4° C. Scintillation counting (Parker, G. J et al. (2000) J. Biomol. Screen., 5: 77-88).
AR (human) (agonist radioligand) LNCaP cells (cytosol)
[³H]methyltrienolone 1 nM 0.8 nM mibolerone (1 μM) 24 h 4° C. Scintillation counting. Zava, D. T et al. (1979) Endocrinology, 104: 1007-1012.

The results are expressed as a percent of control specific binding measured specific binding*100 control specific binding and as a percent inhibition of control specific binding 100-(measured specific binding*100) control specific binding obtained in the presence of compoundn.

The IC₅₀ values (concentration causing a half-maximal inhibition of control specific binding) and Hill coefficients (nH) were determined by non-linear regression analysis of the competition curves generated with mean replicate values using Hill equation curve fitting.

Y=D+[A−D]

1+(C/C50)nH

wherein Y=specific binding, A=left asymptote of the curve, D=right asymptote of the curve, C=compound concentration, C50=IC₅₀, and nH=slope factor. This analysis was performed using software developed at Cerep (Hill software) and validated by comparison with data generated by the commercial software SigmaPlot® 4.0 for Windows® (© 1997 by SPSS Inc.).

The inhibition constants (Ki) are calculated using the Cheng Prusoff equation:

Ki=IC50(1+L/KD)

wherein L=concentration of radioligand in the assay, and KD=affinity of the radioligand for the receptor. A scatchard plot is used to determine the KD.

Radioligand binding inhibition and affinity calculations are determined using [³H]-methyltrienolone, [³H]-dexamethasone and [³H]-estradiol for AR, GR and ER, respectively.

AR=androgen receptor, ER=estrogen receptor, GR=glucocorticoid receptor

### Biological Example 2

### Antagonism of AR (WT or F876L) Reporter Assay

LNCaP AR (cs) and LNCaP F876L luciferase cell lines were generated by transduction of each cell line (description of cell line generation Joseph J D, Lu N, Qian J, Sensintaffar J, Shao G, Brigham D, Moon M, Maneval E C, Chen I, Darimont B, Hager J H. A clinically relevant androgen receptor mutation confers resistance to second-generation antiandrogens enzalutamide and ARN-509. Cancer Discov 2013; 3:1020-1029) with an Androgen Response Element Firefly Luciferase lentiviral construct at an MOI (multiplicity of infection) of 50 following the manufacturer's instructions (Qiagen). A stable pooled-population cell line was generated using puromycin (Life Technologies) selection at 1:10,000 v/v. The protocol below was used for both cell lines and for testing of the compounds of Formula (I) of the present invention.

LNCaP cells were grown to about 80% confluence, media removed and cells rinsed in Hank's balanced salt solution prior to separation from the plate with 0.05% Trypsin EDTA. Cells were lifted and trypsin negated in complete CSS (charcoal stripped serum) culture media. CSS was maintained on cells for 24 h prior to assay, at which time 5,000 cells/20 μL were seeded in Greiner 384 well White/White Tissue Culture Treated Plates and incubated for a further 1-2 hours at 37° C., 5% CO₂, prior to addition of 10 μL, of 4× Test Compounds (compounds described herein) or Assay Controls (all diluted in complete media containing 10% css). A further 10 μL, of 4×R-1881 Agonist Challenge (antagonist assay) or Buffer (agonist assay) was then added (all diluted in complete media containing 10% CSS). Agonist challenge was at 400 pM for WT assay and 600 pM for F876L assay. Plates containing cells and compounds herein were incubated for a further 20-24 hours at 37° C., 5% CO₂ before addition of 40 μL/well of Steady-Glo Luciferase Assay System Reagent (Promega# E2520). After 1 h, plates were read for luminescence on a BMG Pherastar.

### Agonist Challenge:

R-1881 (Metribolone)—Agonist

### Antagonist Control (Low Control):

5-(5-(4-((1-Methylpiperidin-4-yl)oxy)phenyl)-8-oxo-6-thioxo-5,7-diazaspiro[3.4]octan-7-yl)-3-(trifluoromethyl)picolinonitrile (WO 2011/103202, EXAMPLE 19, Compound 129, CAS #1332390-06-3).

### Calculations and Formulae:

RLU results were collected from the Pherastar and used directly for data calculation.

### Percent Max & Inhibition Calculated for Assays:

% Inhibition:

(1−(Sample RLU−Ave Low Control RLU[10 μM Antagonist Control])/(Ave High Control RLU [400 pM R-1881]−Ave Low Control RLU[10 μM Antagonist Control]))*100.

% of 1 μM R-1881 Agonist Max:

((Sample RLU−Ave Low Control RLU[DMSO/Buffer])/(Ave High Control RLU [1 μM R-1881]−Ave Low Control RLU[DMSO/Buffer]))*100.

EC/IC50 calculations were achieved utilizing calculated RLU data and data fitting macros. Data were fit using least-squares methods to the following formula:

$${Y}_{[ \text{?} ]} = {Y}_{[ low cmpd ]} - \frac{( {Y}_{[ high cmpd ]} {Y}_{[ low cmpd ]} ) * {Y}_{[ cmpd ]}^{\text{?}}}{{Y}_{[ cmpd ]}^{Hill} + IC {50}^{ Hill}}$$

$$\text{?} \text{indicates text missing or illegible when filed}$$

wherein

Y_{[low cmpd]}=Y value with inactive compound

Y_{[high cmpd]}=Y value with fully active compound effector

Hill=Hill coefficient

EC/IC₅₀=concentration of compound with 50% effect

Resultant data are shown in Table 3.

TABLE 3

| | LNCaP-AR- | | LNCaP-AR- | | LNCaP-AR- | | LNCaP-AR- | |
|---|---|---|---|---|---|---|---|---|
| | wt ANT | | wt AG | | F876L ANT | | F876L AG | |
| | | MAX | | MAX | | MAX | | MAX |
| Cpd | | % | | % | | % | | % |
| No. | pIC₅₀ | Inh | pEC₅₀ | Stim | pIC₅₀ | Inh | pEC₅₀ | Stim |
| 1 | 6.06 | 100.4 | <4.82 | 0.5 | 6.29 | 100.8 | <4.82 | −0.6 |
| 2 | 5.65 | 98.1 | <4.82 | 0.8 | 6.54 | 99.3 | <4.82 | −0.4 |
| 3 | 5.87 | 99.6 | <4.82 | 0.3 | 6.49 | 98.9 | <4.82 | −0.3 |
| 4 | 5.96 | 100.2 | <4.82 | 0.2 | 6.57 | 100.3 | <4.82 | −0.4 |
| 5 | 6.01 | 100.8 | <4.82 | 0.7 | 6.38 | 98.3 | <4.82 | −0.1 |
| 6 | 6.19 | 100.2 | <4.82 | 0.4 | 6.75 | 99.5 | <4.82 | 0.0 |
| 7 | 6.38 | 100.9 | <4.82 | −0.2 | 6.63 | 100.3 | <4.82 | 0.0 |
| 8 | 5.57 | 98.8 | <4.82 | 0.2 | 6.58 | 92.1 | <4.82 | −0.1 |
| 9 | 6.06 | 99.0 | <4.82 | −0.2 | 6.16 | 100.2 | <4.82 | 0.0 |
| 10 | 6.14 | 99.0 | <4.82 | 0.6 | 6.31 | 98.4 | <4.82 | 0.1 |
| 11 | 6.17 | 101.0 | <4.82 | −0.1 | 6.53 | 99.9 | <4.82 | −0.1 |
| 12 | 5.76 | 98.4 | <4.82 | 0.9 | 6.64 | 98.7 | <4.82 | −0.3 |
| 13 | 6.39 | 102.2 | <4.82 | 0.4 | 6.64 | 101.2 | <4.82 | 0.1 |
| 14 | 7.06 | 101.5 | <4.82 | 0.0 | 6.95 | 99.7 | <4.82 | −0.1 |
| 15 | 6.68 | 103.2 | <4.82 | −0.1 | 7.33 | 102.1 | <4.82 | 0.0 |
| 16 | 6.20 | 99.2 | <4.82 | 0.3 | 6.57 | 98.7 | <4.82 | 0.0 |
| 17 | 5.57 | 95.5 | <4.82 | −0.1 | 5.96 | 97.2 | <4.82 | 0.1 |
| 18 | 6.69 | 101.4 | <4.82 | −0.2 | 6.72 | 100.8 | <4.82 | 0.0 |
| 19 | 6.06 | 99.6 | <4.82 | 0.3 | 6.28 | 98.1 | <4.82 | 0.2 |
| 20 | 5.88 | 90.0 | <4.82 | 1.3 | 6.01 | 95.4 | <4.82 | 0.2 |
| 21 | 6.14 | 100.4 | <4.82 | −0.2 | 6.41 | 98.6 | <4.82 | 0.1 |
| 22 | 5.61 | 99.5 | <4.82 | 0.0 | 6.14 | 99.7 | <4.82 | 0.1 |
| 23 | 5.90 | 99.4 | <4.82 | 0.9 | 6.46 | 99.6 | <4.82 | 0.0 |
| 24 | 6.48 | 100.0 | <4.82 | 0.0 | 6.64 | 99.5 | <4.82 | 0.1 |
| 25 | 6.00 | 98.0 | <4.82 | 1.5 | 6.28 | 99.7 | <4.82 | 0.2 |
| 26 | 6.09 | 97.9 | <4.82 | 1.1 | 6.05 | 97.9 | <4.82 | 0.3 |
| 27 | 6.04 | 96.8 | <4.82 | 0.4 | 6.20 | 98.8 | <4.82 | 0.2 |
| 28 | 6.30 | 99.6 | <4.82 | −0.1 | 6.69 | 100.2 | <4.82 | 0.0 |
| 29 | 6.41 | 102.3 | <4.82 | 0.7 | 7.00 | 100.6 | <4.82 | 0.3 |
| 30 | 6.48 | 100.3 | <4.82 | 0.1 | 6.79 | 98.8 | <4.82 | 0.1 |
| 31 | 5.65 | 98.6 | <4.82 | 0.2 | 5.65 | 91.0 | <4.82 | 0.3 |
| 32 | 6.31 | 102.1 | <4.82 | 0.7 | 6.55 | 101.0 | <4.82 | 0.1 |
| 33 | 5.91 | 99.6 | <4.82 | 0.3 | 6.31 | 100.0 | <4.82 | 0.3 |
| 34 | 6.25 | 99.7 | <4.82 | −0.1 | 6.21 | 99.4 | <4.82 | 0.2 |
| 35 | 6.62 | 97.5 | <4.82 | −0.1 | 6.84 | 101.2 | <4.82 | 0.0 |
| 36 | 5.83 | 96.1 | <4.82 | 0.9 | 6.07 | 91.3 | <4.82 | 0.3 |
| 37 | 5.92 | 99.3 | <4.82 | 0.3 | 6.40 | 96.5 | <4.82 | 0.3 |
| 38 | 6.40 | 100.0 | <4.82 | 1.2 | 6.64 | 99.0 | <4.82 | 0.3 |
| 39 | 5.74 | 97.2 | <4.82 | 0.2 | 5.81 | 94.4 | <4.82 | 0.3 |
| 40 | 6.62 | 101.2 | <4.82 | −0.3 | 6.79 | 99.5 | <4.82 | 0.2 |
| 41 | 6.55 | 102.5 | <4.82 | −0.2 | 6.73 | 101.6 | <4.82 | 0.3 |
| 42 | 6.61 | 100.5 | <4.82 | 0.3 | 6.51 | 99.8 | <4.82 | 0.0 |
| 43 | 6.30 | 97.3 | <4.82 | 0.7 | 6.61 | 98.9 | <4.82 | 0.4 |
| 44 | 6.71 | 102.9 | <4.82 | −0.2 | 6.75 | 101.1 | <4.82 | 0.0 |
| 45 | 5.99 | 101.2 | <4.82 | 0.2 | 6.36 | 100.3 | <4.82 | 0.1 |
| 46 | 6.05 | 99.9 | <4.82 | −0.1 | 6.20 | 98.3 | <4.82 | 0.2 |
| 47 | 6.61 | 101.3 | <4.82 | 0.9 | 6.71 | 99.6 | <4.82 | 0.4 |
| 48 | 6.21 | 96.3 | <4.82 | 1.1 | 6.40 | 99.2 | <4.82 | 0.5 |
| 49 | 6.30 | 100.9 | <4.82 | 0.6 | 6.59 | 96.5 | <4.82 | 0.5 |
| 50 | 6.44 | 98.0 | <4.82 | 0.7 | 6.55 | 98.2 | <4.82 | 0.2 |
| 51 | 6.41 | 99.0 | <4.82 | 0.8 | 6.70 | 100.7 | <4.82 | 0.2 |
| 52 | 6.13 | 100.7 | <4.82 | 0.4 | 6.33 | 101.6 | <4.82 | 0.4 |
| 53 | 6.04 | 97.0 | <4.82 | 1.0 | 6.19 | 96.7 | <4.82 | 0.5 |
| 54 | 6.42 | 100.9 | <4.82 | 0.1 | 6.66 | 98.9 | <4.82 | 0.5 |
| 55 | 6.09 | 101.0 | <4.82 | 0.7 | 6.50 | 100.3 | <4.82 | 0.6 |
| 56 | 6.07 | 99.7 | <4.82 | 0.5 | 6.08 | 97.8 | <4.82 | 0.4 |
| 57 | 6.49 | 99.0 | <4.82 | 0.0 | 6.32 | 95.5 | <4.82 | 0.5 |
| 58 | 6.61 | 102.0 | <4.82 | 0.1 | 6.69 | 98.2 | <4.82 | 0.6 |
| 59 | 5.89 | 102.3 | <4.82 | 1.5 | 6.17 | 100.4 | <4.82 | 0.7 |
| 60 | 6.03 | 98.7 | <4.82 | 1.1 | 6.28 | 99.3 | <4.82 | 0.7 |
| 61 | 5.90 | 100.1 | <4.82 | 1.1 | 6.42 | 100.4 | <4.82 | 0.7 |
| 62 | 6.47 | 98.8 | <4.82 | 0.8 | 7.04 | 99.7 | <4.82 | 0.4 |
| 63 | 6.11 | 68.6 | <4.82 | 0.7 | 6.66 | 83.4 | <4.82 | 0.3 |
| 64 | 6.55 | 98.9 | <4.82 | 1.2 | 6.76 | 98.1 | <4.82 | 0.7 |
| 65 | 6.38 | 97.8 | <4.82 | 1.2 | 6.41 | 98.9 | <4.82 | 0.8 |
| 66 | 5.94 | 98.9 | <4.82 | 0.6 | 6.22 | 96.2 | <4.82 | 0.1 |
| 67 | 5.90 | 99.2 | <4.82 | 1.5 | 6.39 | 97.4 | <4.82 | 0.8 |
| 68 | 6.23 | 96.7 | <4.82 | 0.2 | 6.37 | 98.0 | <4.82 | 0.9 |
| 69 | 6.55 | 101.5 | <4.82 | 0.9 | 6.88 | 99.1 | <4.82 | 0.7 |
| 70 | 6.13 | 99.2 | <4.82 | 0.3 | 6.29 | 99.5 | <4.82 | 0.9 |
| 71 | 6.67 | 98.8 | <4.82 | 0.5 | 6.60 | 97.5 | <4.82 | 0.9 |
| 72 | 6.60 | 102.0 | <4.82 | 0.3 | 6.99 | 100.7 | <4.82 | 0.6 |
| 73 | 6.12 | 99.5 | <4.82 | 1.9 | 6.38 | 99.1 | <4.82 | 0.6 |
| 74 | 5.94 | 94.1 | <4.82 | 0.6 | 6.12 | 93.8 | <4.82 | 1.0 |
| 75 | 6.25 | 101.3 | <4.82 | 0.8 | 6.84 | 100.4 | <4.82 | 0.8 |
| 76 | 5.84 | 97.8 | <4.82 | 1.9 | 6.07 | 91.6 | <4.82 | 1.0 |
| 77 | 6.69 | 99.5 | <4.82 | 0.8 | 6.74 | 100.3 | <4.82 | 0.6 |
| 78 | 5.88 | 96.5 | <4.82 | 0.7 | 5.98 | 95.9 | <4.82 | 1.0 |
| 79 | 6.20 | 100.6 | <4.82 | 1.6 | 6.48 | 100.7 | <4.82 | 0.5 |
| 80 | 6.05 | 100.2 | <4.82 | 0.4 | 6.37 | 95.4 | <4.82 | 1.2 |
| 81 | 6.15 | 95.9 | <4.82 | 0.7 | 6.11 | 100.0 | <4.82 | 1.2 |
| 82 | 6.44 | 102.6 | <4.82 | 0.0 | 6.36 | 95.5 | <4.82 | 1.2 |
| 83 | 5.65 | 98.8 | <4.82 | 0.1 | 6.00 | 99.5 | <4.82 | 0.5 |
| 84 | 6.04 | 87.8 | <4.82 | 1.0 | 6.32 | 91.9 | <4.82 | 1.3 |
| 85 | 6.73 | 101.1 | <4.82 | 0.2 | 7.01 | 99.8 | <4.82 | 0.8 |
| 86 | 6.59 | 91.7 | <4.82 | 0.6 | 6.68 | 93.3 | <4.82 | 1.3 |
| 87 | 6.51 | 100.6 | <4.82 | 1.1 | 6.54 | 99.7 | <4.82 | 0.9 |
| 88 | 6.20 | 97.2 | <4.82 | 0.7 | 6.86 | 87.3 | <4.82 | 0.7 |
| 89 | 6.20 | 100.7 | <4.82 | 0.3 | 6.36 | 96.2 | <4.82 | 1.4 |
| 90 | 6.29 | 100.0 | <4.82 | 0.5 | 6.20 | 98.6 | <4.82 | 1.4 |
| 91 | 6.27 | 98.5 | <4.82 | 1.1 | 6.52 | 98.6 | <4.82 | 1.4 |
| 92 | 6.20 | 94.7 | <4.82 | 2.8 | 6.59 | 98.2 | <4.82 | 1.3 |
| 93 | 6.39 | 100.3 | <4.82 | 0.9 | 6.63 | 100.1 | <4.82 | 0.8 |
| 94 | 6.14 | 95.0 | <4.82 | 6.0 | 6.20 | 99.2 | <4.82 | 1.5 |
| 95 | 6.10 | 96.1 | <4.82 | 0.2 | 5.98 | 92.3 | <4.82 | 1.5 |
| 96 | 6.39 | 101.3 | <4.82 | 1.5 | 6.46 | 100.3 | <4.82 | 0.8 |
| 97 | 6.04 | 100.2 | <4.82 | 0.7 | 6.17 | 99.6 | <4.82 | 1.6 |
| 98 | 6.53 | 101.6 | <4.82 | 0.0 | 6.61 | 98.9 | <4.82 | 1.6 |
| 99 | 6.38 | 97.2 | <4.82 | 1.6 | 6.41 | 85.9 | <4.82 | 1.6 |
| 100 | 6.38 | 102.1 | <4.82 | 1.8 | 6.59 | 97.2 | <4.82 | 1.7 |
| 101 | 6.32 | 99.4 | <4.82 | 1.1 | 6.44 | 95.3 | <4.82 | 1.7 |
| 102 | 6.19 | 101.3 | <4.82 | 0.9 | 6.30 | 99.6 | <4.82 | 1.9 |
| 103 | 6.25 | 29.0 | <4.82 | 4.0 | 6.97 | 64.9 | <4.82 | 2.0 |
| 104 | 6.89 | 99.7 | <4.82 | 2.7 | 7.92 | 98.4 | <4.82 | 2.2 |
| 105 | 6.24 | 95.7 | <4.82 | 1.2 | 6.31 | 95.3 | <4.82 | 2.3 |
| 106 | 6.21 | 100.7 | <4.82 | 1.8 | 6.20 | 95.9 | <4.82 | 2.4 |
| 107 | 6.25 | 89.1 | <4.82 | 2.1 | 6.79 | 97.3 | <4.82 | 2.4 |
| 108 | 6.09 | 94.5 | <4.82 | 2.3 | 6.41 | 95.6 | <4.82 | 2.5 |
| 109 | 5.71 | 97.9 | <4.82 | 0.2 | 6.13 | 98.4 | <4.82 | 1.1 |
| 110 | 6.71 | 100.5 | <4.82 | 1.3 | 6.84 | 100.2 | <4.82 | 2.6 |
| 111 | 6.39 | 102.2 | <4.82 | 0.5 | 6.28 | 94.1 | <4.82 | 2.7 |
| 112 | 6.45 | 100.5 | <4.82 | 1.1 | 6.48 | 97.0 | <4.82 | 2.7 |
| 113 | 5.93 | 97.4 | <4.82 | 25.3 | 6.06 | 98.2 | <4.82 | 3.1 |
| 114 | 6.62 | 98.7 | <4.82 | 0.7 | 6.45 | 97.8 | <4.82 | 3.3 |
| 115 | 6.43 | 100.0 | <4.82 | 1.1 | 6.36 | 96.1 | <4.82 | 3.4 |
| 116 | 5.59 | 95.3 | <4.82 | 1.1 | 6.20 | 98.6 | <4.82 | 3.5 |
| 117 | 5.90 | 100.1 | <4.82 | 0.7 | 6.54 | 96.4 | <4.82 | 3.5 |
| 118 | 5.96 | 101.1 | <4.82 | 0.4 | 6.80 | 99.4 | <4.82 | 3.9 |
| 119 | 6.95 | 97.7 | <4.82 | 2.2 | 6.80 | 96.0 | <4.82 | 3.9 |
| 120 | 6.44 | 100.1 | <4.82 | 1.2 | 6.65 | 98.8 | <4.82 | 3.9 |
| 121 | 5.56 | 93.6 | <4.82 | 1.2 | 6.24 | 98.7 | <4.82 | 3.0 |
| 122 | 6.52 | 99.7 | <4.82 | 2.2 | 6.36 | 90.5 | <4.82 | 6.2 |

As used herein:
pIC₅₀ is defined as −Log₁₀(IC₅₀ expressed in [Molar]).
pEC₅₀ is defined as −Log₁₀(EC₅₀ expressed in [Molar]).
MAX % Inh is defined as the maximum % inhibition of R1881 control response observed for a compound over the tested concentration range.
MAX % Stim is defined as the maximum % stimulation (agonist response) observed for a compound over the tested concentration range.
LNCaP-AR-wt ANT refers to the reporter assay using LNCaP cells stably transfected with the Androgen Response Element Firefly Luciferase lentiviral construct and wild-type Androgen Receptor (AR-wt) in Antagonist mode.
LNCaP-AR-wt AG refers to the reporter assay using LNCaP cells stably transfected with the Androgen Response Element Firefly Luciferase lentiviral construct and wild-type Androgen Receptor (AR-wt) in Agonist mode.
LNCaP-AR-F876L ANT refers to the reporter assay using LNCaP cells stably transfected with the Androgen Response Element Firefly Luciferase lentiviral construct and F876L mutant Androgen Receptor (AR-F876L) in Antagonist mode.
LNCaP-AR-F876L AG refers to the reporter assay using LNCaP cells stably transfected with the Androgen Response Element Firefly Luciferase lentiviral construct and F876L mutant Androgen Receptor (AR-F876L) in Agonist mode.

### Biological Example 2

### AR in Cell Western Assay

LNCaP cells (8,000/well) are plated in RPMI media containing 10% Charcoal Dextran Stripped Serum into plates coated with poly-d-lysine. After 24 h cells are treated with compound from 30 μM to 0.0003 μM. At 20 h post compound addition the cells were fixed (30% formaldehyde in PBS) for 20′. Cells are permeabilized in PBS 0.1% Triton (50 μL/well, three times for 5′ each) and blocked with LiCor blocking buffer (50 μL/well, 90′). The wells are then incubated overnight at 4° C. with the rabbit IgG androgen receptor antibody (AR-N20, Santa Cruz antibody) diluted 1:1000 in LiCor blocking buffer/0.1% Tween-20. Wells are washed with 0.1% Tween-20/PBS (50 μL/well, 5′ each) and then incubated in goat anti-rabbit IRDye™ 800 CW (1:1000) and DRAQ5 DNA dye (1:10,0000 for 5 mM stock) diluted in 0.2% Tween-20/0.01% SDS/LiCor blocking buffer in the dark (90′). Cells are washed (50 μL/well, 5′ each) in 0.1% Tween-20/PBS. Wash buffer is removed and plates were read using the LiCor Odyssey.

### Biological Example 4

### LNCaP AR Localization Assay

LNCaP cells are seeded on day 1 in plates and incubated overnight at 37° C. prior to addition of 20 μL pre-diluted compound or DMSO (basal, vehicle control). Plates are incubated at 37° C. for 1-2 h before addition of 20 μL of ligand solution (antagonist mode, high control) or CSS medium (agonist mode, unstimulated control) and incubation of the cells for +/−24 h.

Cells are fixed in 140 μL of 10% Formaldehyde (5% final) and plates incubated for 15-20 min at RT. 100 μL 100% ice cold Methanol (stored at −20° C.) is added to permeabilise the cells, antibody staining protocol initiated and plates prepared for imaging. Staining is performed using an indirect immunofluorescence assay: for AR, primary antibody is a specific mouse anti-AR antibody (ab49450, Abcam), followed by a secondary goat anti-mouse antibody, carrying an alexa 488 fluorophore; for PSA, primary antibody is a specific rabbit anti-PSA antibody (5365S, Cell Signaling Technology), followed by a secondary goat anti rabbit antibody, carrying an alexa 568 fluorophore. Cells are counterstained with Hoechst for the nucleus and cytoplasmic stain for the cytoplasm stain. Plates are washed and maintained in PBS at 4° C. until further processed.
Plates are imaged using the 20×W lens on the Opera (Perkin Elmer) and the following calculations are then applied to derive the reported data from this assay

LC=median of the low control values=minimum translocation

=cells in CSS medium(0,5% DMSO) and showing minimum translocation

HC=median of the high control values=maximum translocation

=cells in CSS medium containing 1 nM of R1881 ligand(0,5% DMSO)

% EFFECT=(sample−LC)/(HC−LC)*100%

CTL=% of high−controls=(sample/HC)*100

Several features are calculated but include:
Ratio_Nuc2_Cell_AR_TotalIntBC.median: % of total AR in the nucleus calculated as “total nuclear AR intensity”/“total cellular AR intensity” on the single-cell level and then the median over all cells reported as well feature [% effect]
Cell_AR_MeanIntBC.median: AR levels in the whole cell [% effect]
Cyto_AR_meanIntBC.median: AR levels in cytoplasm [% effect]
Nuc_AR_MeanIntBC.median: AR levels in nucleus [% effect]
Cell_Rpt_MeanIntBC.median: PSA levels in whole cell [% effect]
CellCount_AllDetected: number of the cells

### Biological Example 5

### Prostate Cancer Cell Viability Assay-VCaP

VCaP cells are counted and seeded into black 384-well plates with clear bottoms at a concentration of 125,000 cells per mL in phenol red-free DMEM containing 10% Charcoal Stripped Serum. 16 μL of the suspension is added per well and incubated for 48 h to allow the cells to adhere. After 48 hours, a 12 point serial semilog dilution of each compound is added to the cells in 16 μL at a final concentration of 100 μM to 0.0003 μM. The compounds of Formula (I) are also run in antagonist mode using 30 pM R1881 in which 8 μL of the compound is added to the cells followed by 8 μL of R1881. After 5 days of incubation at 37° C., 16 μL Of CellTiter-Glo (Promega) are added to the cells and the relative luminescence units (RLUs) of each well determined using the Envision. The percent stimulation and % inhibition are determined for each sample and plotted using GraphPad Prism.

### Biological Example 6

### LNCaP Proliferation Assays

LNCaP cells are expanded in RPMI 10% FBS in T150 flasks. The cells are dislodged with 0.25% Trypsin, washed in complete media, centrifuged (300 g, 3 min), and the supernatant aspirated. The cells are resuspended in RPMI phenol-red free media with 1% charcoal-stripped serum (CSS) and counted using a ViCELL (Beckman-Coulter). 7500 cells are added to each well of a white optical bottom 384-well plate and are incubated for 2 days at 37° C. 5% CO₂. Compound dilutions are prepared in RPMI CSS using 50 mM stock solutions and added to the cells either alone (agonist mode) or in combination with 0.1 nM R1881 (antagonist mode). The plates are incubated for 4 days, followed by addition of CellTiter-Glo Luminescent Cell Viability kit reagent (Promega). The plates are placed on a shaker at 3000 rpm for 10 minutes and then read on an EnVision plate reader (Perkin Elmer) using Luminescence assay default settings. The data are analyzed, normalized to 0.1 nM R1881 stimulation, and plotted in GraphPad Prism.

### Biological Example 7

### Luciferase Transcriptional Reporter Assays (WT and Mutant AR)

HepG2 cells were maintained in EMEM supplemented with 10% FBS. One day before transfection, the media was changed to EMEM with 10% CSS. T-150 flasks were transiently transfected using 120 μL Lipofectamine 2000 (Life Technologies), 30 μg mutant cDNA (expression vector)—mutant cDNA tested were L701H, T877A, W741C and H874Y—and 40 μg 4×ARE-Luciferase (reporter vector) in OptiMEM and the flasks were incubated overnight. Cells were then trypsinized, counted and resuspended at 500,000 cells/mL. For agonist mode, the compounds of Formula (I) are serially diluted and 50 μL of the compound added per well. 50 μL of the cells are added to each well and incubated for 48 hours. For antagonist mode, a final concentration of 90 pM R1881 was added to the diluted compounds and incubated for 48 hours. The plates were then assayed using SteadyGlo and read on the Envision. Percent Stimulation and Inhibition is determined and analyzed using GraphPad Prism. Resultant data are shown in Table 7.

TABLE 7
Summary of Antagonist Activity, IC₅₀ for compounds of
Formula (I) in AR Mutant Reporter Assays.

Antagonism; IC₅₀ μM [% Eₘₐₓ]
AR construct

| Compound | L701H | T877A | W741C | H874Y |
|---|---|---|---|---|
| 8 | NT | NT | NT | NT |
| 35 | 3.36 | 3.59 | 5.79 | 1.95 |
| 40 | NT | 7.51* | NT | NT |
| 60 | NT | NT | NT | NT |
| 85 | 8.86* | 11.2* | 14.0 | 9.48* |

The antagonistic (IC₅₀) values for each of the AR cDNA used in the reporter assays are summarized. NT is not tested * denotes incomplete inhibition (otherwise 100%).
All values are calculated relative to the activity of R1881 induced androgen receptor activity (n≧3).

### Biological Example 8

### AR-VP16 DNA Binding Assays

HepG2 cells were maintained in EMEM supplemented with 10% FBS. One day before transfection, the media was changed to EMEM with 10% CSS. T-150 flasks were transiently transfected using 120 μL Lipofectamine 2000 (Life Technologies), 24.5 μg AR-VP16 or F876L-VP16 (expression vector) and 49 μg 4×ARE-Luciferase (reporter vector) in OptiMEM and the flasks were incubated overnight. Cells were then trypsinized, counted and resuspended at 500,000 cells/mL. For agonist mode, the compounds were serially diluted and 50 μL of the compound was added per well. 50 μL of the cells were added to each well and incubated for 48 hours. For antagonist mode, a final concentration of 90 pM (VP16 AR) or 1 nM (VP16 F876L) R1881 was added to the plate and incubated for 48 hours. The plates were then assayed using SteadyGlo and read on the Envision. Percent Stimulation and Inhibition were determined and analyzed using GraphPad Prism. Resultant data are shown in Table 8.

TABLE 8

IC₅₀ (μM)

| Compound | VP16 WT | VP16 F876L |
|---|---|---|
| 8 | 0.121 | 15.35 |
| 35 | 3.39 | 2.74 |
| 40 | 0.024* | 0.994* |
| 60 | 2.05 | 0.746 |
| 85 | 0.96 | 0.127 |

### Biological Example 9

### GABA-Gated C1 Channel Antagonist Radioligand Binding Assay

GABA-gated C1 Chanel assays are performed at CEREP according to the following method. Membrane homogenates of cerebral cortex (120 μg protein) are incubated for 120 min at 22° C. with 3 nM [³⁵5]-TBPS in the absence or presence of the test compound in a buffer containing 50 mM Na₂HPO₄/KH₂PO₄ (pH 7.4) and 500 mM NaCl. Nonspecific binding is determined in the presence of 20 μM picrotoxinin. Following incubation, the samples are filtered rapidly under vacuum through glass fiber filters (GF/B, Packard) presoaked with 0.3% PEI and rinsed several times with ice-cold 50 mM Tris-HCl using a 96-sample cell harvester (Unifilter, Packard). The filters are dried then counted for radioactivity in a scintillation counter (Topcount, Packard) using a scintillation cocktail (Microscint 0, Packard). The results are expressed as a percent inhibition of the control radio ligand specific binding. The standard reference compound is picrotoxinin, which is tested in each experiment at several concentrations to obtain a competition curve from which its IC₅₀ was calculated.

### In-Vivo Assays

### Biological Example V1

### Hershberger Assay

The effect of AR antagonists on androgen dependent signaling in vivo was assessed using the Hershberger assay. In this assay, peripubertal castrated male Sprague-Dawley rats were administered AR antagonists described herein in the presence of testosterone (0.4 mg/kg testosterone propionate) and the weights of androgen dependent organs measured. Dosing was continued for 10 days and measurements taken 24 h after the last dose. The extent of antagonism of AR and consequent inhibition of organ growth was evaluated by comparison to the castration control. Compounds of Formula (I) were dosed orally QD and an endpoint assessment made by change in weight of 5 androgen sensitive organs (ASO): Paired Cowper's Glands (CG), Seminal Vesicles with Fluids and Coagulating Glands (SVCG), Glans Penis (GP), Ventral Prostate (VP) and Levator Ani-Bulbocavernosus Complex (LABC)). According to assay guidelines, statistically significant suppression of ASO is required in 2 of 5 organs for a compound to be classified as an anti-androgen (analysis was performed by t-test/Mann-Whitney).

Compounds defined herein were administered at the indicated dose (mg/kg) and flutamide (FT), positive control, at 3 mg/kg. All compounds were co-administered with testosterone propionate (TP, 0.4 mg/kg) which was also administered alone, untreated control, (castrated only rats served as the control for complete androgen blockade). A statistically significant change in ASO achieved in at least 2 of 5 organs was indicative of an active compound. Administration of Compound 43 resulted in significant reduction in ASO versus TP control (p≦0.05) in all 5 organs. Data for the inhibition of growth of the Seminal Vesicle and Coagulating Glands (SVCG) and Ventral Prostate (VP) was reported for all studies (mean organ weight (% of TP control)±SD (n=6)). Resultant data are shown in Table 10.

TABLE 10

| | |
|---|---|
| | ASO Organ Growth |
| Compound | (% of TP control) |

| [dose] | SVCG | VP |
|---|---|---|
| Flutamide (+ve control) | 16.6 ± 16.3 | 24.4 ± 35.5 |
| [3 mg/kg] | | |
| Compound 35 | 28.1 ± 19.5 | 25.7 ± 22.9 |
| [30 mg/kg] | | |
| Flutamide (+ve control) | 22.5 ± 22.9 | 31.1 ± 29.2 |
| [3 mg/kg] | | |
| Compound 85 | 67.3 ± 20.2 | 72.3 ± 18.5 |
| [3 mg/kg] | | |
| Compound 85 | 31.6 ± 37.7 | 37.4 ± 25.6 |
| [10 mg/kg] | | |
| Compound 85 | 8.5 ± 14.7 | 13.2 ± 19.2 |
| [30 mg/kg] | | |

### Biological Example V2

### Castrate Resistant Prostate Cancer Xenograft Studies

Castrate six to seven week old male SCID Hairless Outbred mice (SHO, Charles Rivers Laboratories) were used as the host strain for xenograft studies. LNCaP SRα F876L tumors were established in host mice and the anti-tumor activity of compounds defined herein was determined. Dosing was initiated when tumors reached 100 to 200 mm³ and animals were randomized to each of test groups (vehicle (HP-β-CD), 10 mg/kg, 30 mg/kg or 50 mg/kg compound). Compound was dosed orally, QD, for 28 days and tumor size was measured twice weekly along with body weight measurement. At the end of study, the TGI was calculated using initial tumor volume and final tumor volume measurements. TGI: 100−(Treated/Control*100). At the termination of study tumors were collected and stored for further analyses. Resultant data are shown in Table 11.

TABLE 11

TGI (%)

| Compound | 10 mg/kg | 30 mg/kg |
|---|---|---|
| Compound 35 | | 53 |
| Compound 85 | 80.2 | 100.8 |

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

We claim:

1. A method for treating and/or ameliorating diseases, syndromes, disorders, or conditions associated with AR mutant receptors linked to castration-resistant prostate cancer, in a subject, including a mammal and/or human, in need thereof, who has demonstrated resistance to a first or second generation AR antagonist, comprising, consisting of, and/or consisting essentially of, administering to a subject in need thereof, a therapeutically effective amount of a compound of Formula (I) 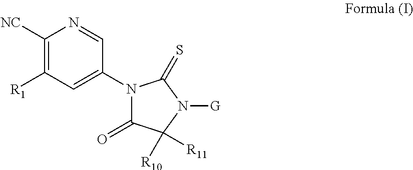
or a pharmaceutically acceptable salt form thereof,
selected from the group consisting of
5-[4,4-dimethyl-3-[4-[(1-methyl-4-piperidyl)oxy]phenyl]-5-oxo-2-thioxo-imidazolidin-1-yl]-3-methyl-pyridine-2-carbonitrile;
4-[6-(6-cyano-5-methyl-3-pyridyl)-5-oxo-7-thioxo-6,8-diazaspiro[3.4]octan-8-yl]-2-fluoro-N-tetrahydropyran-4-yl-benzamide;
4-[6-(6-cyano-5-methyl-3-pyridyl)-5-oxo-7-thioxo-6,8-diazaspiro[3.4]octan-8-yl]-2-fluoro-N-(tetrahydropyran-4-ylmethyl)benzamide;
3-methyl-5-[8-[4-[(1-methyl-4-piperidyl)oxy]phenyl]-5-oxo-7-thioxo-6,8-diazaspiro[3.4]octan-6-yl]pyridine-2-carbonitrile;
4-[6-(6-cyano-5-methyl-3-pyridyl)-5-oxo-7-thioxo-6,8-diazaspiro[3.4]octan-8-yl]-N,2-dimethyl-benzamide;
5-[8-[4-(1,1-dioxothian-4-yl)oxyphenyl]-5-oxo-7-thioxo-6,8-diazaspiro[3.4]octan-6-yl]-3-methyl-pyridine-2-carbonitrile;
5-[8-(7-isoquinolyl)-5-oxo-7-thioxo-6,8-diazaspiro[3.4]octan-6-yl]-3-(trifluoromethyl)pyridine-2-carbonitrile;
5-[5-oxo-8-(4-piperazin-1-ylphenyl)-7-thioxo-6,8-diazaspiro[3.4]octan-6-yl]-3-(trifluoromethyl)pyridine-2-carbonitrile
5-[5-oxo-8-(3-pyridyl)-7-thioxo-6,8-diazaspiro[3.4]octan-6-yl]-3-(trifluoromethyl)pyridine-2-carbonitrile;
3-methyl-5-[8-[4-[2-(4-methylpiperazin-1-yl)ethoxy]phenyl]-5-oxo-7-thioxo-6,8-diazaspiro[3.4]octan-6-yl]pyridine-2-carbonitrile;
4-[6-(6-cyano-5-methyl-3-pyridyl)-5-oxo-7-thioxo-6,8-diazaspiro[3.4]octan-8-yl]-2-fluoro-N-(2-fluorophenyl)benzamide;
4-[3-(6-cyano-5-methyl-3-pyridyl)-5,5-dimethyl-4-oxo-2-thioxo-imidazolidin-1-yl]-2-fluoro-N-methyl-benzamide;
3-methyl-5-[5-oxo-8-(4-tetrahydrothiopyran-4-yloxyphenyl)-7-thioxo-6,8-diazaspiro[3.4]octan-6-yl]pyridine-2-carbonitrile;
4-[6-[6-cyano-5-(trifluoromethyl)-3-pyridyl]-5-oxo-7-thioxo-6,8-diazaspiro[3.4]octan-8-yl]-2-fluoro-N-[2-(4-pyridyl)ethyl]benzamide;
4-[6-[6-cyano-5-(trifluoromethyl)-3-pyridyl]-5-oxo-7-thioxo-6,8-diazaspiro[3.4]octan-8-yl]-2-fluoro-N-[2-(2-pyridyl)ethyl]benzamide;
4-[6-(6-cyano-5-methyl-3-pyridyl)-5-oxo-7-thioxo-6,8-diazaspiro[3.4]octan-8-yl]-2-fluoro-N-methyl-benzamide;
4-[6-[6-cyano-5-(trifluoromethyl)-3-pyridyl]-5-oxo-7-thioxo-6,8-diazaspiro[3.4]octan-8-yl]-2-fluoro-N-(2-hydroxy-2-methyl-propyl)benzamide;
5-[8-(2-naphthyl)-5-oxo-7-thioxo-6,8-diazaspiro[3.4]octan-6-yl]-3-(trifluoromethyl)pyridine-2-carbonitrile;
5-[4,4-dimethyl-3-[4-[(1-methyl-4-piperidyl)oxy]phenyl]-5-oxo-2-thioxo-imidazolidin-1-yl]-3-(trifluoromethyl)pyridine-2-carbonitrile;
5-[5-oxo-8-(3-phenoxyphenyl)-7-thioxo-6,8-diazaspiro[3.4]octan-6-yl]-3-(trifluoromethyl)pyridine-2-carbonitrile;
4-[6-[6-cyano-5-(trifluoromethyl)-3-pyridyl]-5-oxo-7-thioxo-6,8-diazaspiro[3.4]octan-8-yl]-N-(cyclopentylmethyl)-2-fluoro-benzamide;
4-[6-[6-cyano-5-(trifluoromethyl)-3-pyridyl]-5-oxo-7-thioxo-6,8-diazaspiro[3.4]octan-8-yl]-2-fluoro-N-(2-morpholinoethyl)benzamide;
4-[6-[6-cyano-5-(trifluoromethyl)-3-pyridyl]-5-oxo-7-thioxo-6,8-diazaspiro[3.4]octan-8-yl]-2-fluoro-N-isopropyl-benzamide;
4-[6-[6-cyano-5-(trifluoromethyl)-3-pyridyl]-5-oxo-7-thioxo-6,8-diazaspiro[3.4]octan-8-yl]-2-fluoro-N-(3-methoxypropyl)benzamide;
4-[6-[6-cyano-5-(trifluoromethyl)-3-pyridyl]-5-oxo-7-thioxo-6,8-diazaspiro[3.4]octan-8-yl]-N-methyl-benzenesulfonamide;
4-[6-[6-cyano-5-(trifluoromethyl)-3-pyridyl]-5-oxo-7-thioxo-6,8-diazaspiro[3.4]octan-8-yl]-2-fluoro-N-[(5-methyl-2-furyl)methyl]benzamide;
4-[6-[6-cyano-5-(trifluoromethyl)-3-pyridyl]-5-oxo-7-thioxo-6,8-diazaspiro[3.4]octan-8-yl]-2-fluoro-N-isopentyl-benzamide;
5-[8-[3-fluoro-4-[(1-methyl-4-piperidyl)oxy]phenyl]-5-oxo-7-thioxo-6,8-diazaspiro[3.4]octan-6-yl]-3-(trifluoromethyl)pyridine-2-carbonitrile;
3-methyl-5-[5-oxo-8-(p-tolyl)-7-thioxo-6,8-diazaspiro[3.4]octan-6-yl]pyridine-2-carbonitrile;
5-[8-[4-[(4-methylpiperazin-1-yl)methyl]phenyl]-5-oxo-7-thioxo-6,8-diazaspiro[3.4]octan-6-yl]-3-(trifluoromethyl)pyridine-2-carbonitrile;
N-[(2-chlorophenyl)methyl]-4-[6-[6-cyano-5-(trifluoromethyl)-3-pyridyl]-5-oxo-7-thioxo-6,8-diazaspiro[3.4]octan-8-yl]-2-fluoro-benzamide;
5-[8-[3-fluoro-4-[2-(2-pyridyl)ethoxy]phenyl]-5-oxo-7-thioxo-6,8-diazaspiro[3.4]octan-6-yl]-3-methyl-pyridine-2-carbonitrile;
4-[6-(6-cyano-5-methyl-3-pyridyl)-5-oxo-7-thioxo-6,8-diazaspiro[3.4]octan-8-yl]-2-fluoro-N-(2-thienylmethyl)benzamide;
5-[8-(1-naphthyl)-5-oxo-7-thioxo-6,8-diazaspiro[3.4]octan-6-yl]-3-(trifluoromethyl)pyridine-2-carbonitrile;
5-[5-oxo-8-[4-(4-piperidyloxy)phenyl]-7-thioxo-6,8-diazaspiro[3.4]octan-6-yl]-3-(trifluoromethyl)pyridine-2-carbonitrile;
N-benzyl-4-[6-[6-cyano-5-(trifluoromethyl)-3-pyridyl]-5-oxo-7-thioxo-6,8-diazaspiro[3.4]octan-8-yl]-2-fluoro-benzamide;
4-[6-[6-cyano-5-(trifluoromethyl)-3-pyridyl]-5-oxo-7-thioxo-6,8-diazaspiro[3.4]octan-8-yl]-2-fluoro-N-[3-(2-oxopyrrolidin-1-yl)propyl]benzamide;
5-[5-oxo-7-thioxo-8-[4-(trifluoromethoxy)phenyl]-6,8-diazaspiro[3.4]octan-6-yl]-3-(trifluoromethyl)pyridine-2-carbonitrile;
4-[6-[6-cyano-5-(difluoromethyl)-3-pyridyl]-5-oxo-7-thioxo-6,8-diazaspiro[3.4]octan-8-yl]-2-fluoro-N-methyl-benzamide;
4-[6-[6-cyano-5-(trifluoromethyl)-3-pyridyl]-5-oxo-7-thioxo-6,8-diazaspiro[3.4]octan-8-yl]-2-fluoro-N-(3-hydroxypropyl)benzamide;
ethyl 2-[[4-[6-[6-cyano-5-(trifluoromethyl)-3-pyridyl]-5-oxo-7-thioxo-6,8-diazaspiro[3.4]octan-8-yl]-2-fluoro-benzoyflamino]acetate;
4-[6-[6-cyano-5-(trifluoromethyl)-3-pyridyl]-5-oxo-7-thioxo-6,8-diazaspiro[3.4]octan-8-yl]-2-fluoro-N-phenethyl-benzamide;
5-[8-[4-[3-(4-methylpiperazin-1-yl)propyl]phenyl]-5-oxo-7-thioxo-6,8-diazaspiro[3.4]octan-6-yl]-3-(trifluoromethyl)pyridine-2-carbonitrile;
4-[6-[6-cyano-5-(trifluoromethyl)-3-pyridyl]-5-oxo-7-thioxo-6,8-diazaspiro[3.4]octan-8-yl]-2-fluoro-N-(2-pyridylmethyl)benzamide;
4-[6-(6-cyano-5-methyl-3-pyridyl)-5-oxo-7-thioxo-6,8-diazaspiro[3.4]octan-8-yl]-2-fluoro-N-(2-methylpyrazol-3-yl)benzamide;
4-[6-[6-cyano-5-(trifluoromethyl)-3-pyridyl]-5-oxo-7-thioxo-6,8-diazaspiro[3.4]octan-8-yl]-2-fluoro-N-(2-methoxyethyl)benzamide;
5-[8-(2-fluoro-4-hydroxy-phenyl)-5-oxo-7-thioxo-6,8-diazaspiro[3.4]octan-6-yl]-3-(trifluoromethyl)pyridine-2-carbonitrile;
5-[8-(4-hydroxyphenyl)-5-oxo-7-thioxo-6,8-diazaspiro[3.4]octan-6-yl]-3-methyl-pyridine-2-carbonitrile;
5-[8-[2-fluoro-4-[(1-methyl-4-piperidyl)oxy]phenyl]-5-oxo-7-thioxo-6,8-diazaspiro[3.4]octan-6-yl]-3-(trifluoromethyl)pyridine-2-carbonitrile;
3-methyl-5-[8-[4-(5-methyl-2-furyl)phenyl]-5-oxo-7-thioxo-6,8-diazaspiro[3.4]octan-6-yl]pyridine-2-carbonitrile;
5-[8-[4-[[1-(2-hydroxyethyl)-4-piperidyl]oxy]phenyl]-5-oxo-7-thioxo-6,8-diazaspiro[3.4]octan-6-yl]-3-(trifluoromethyl)pyridine-2-carbonitrile;
5-[8-[3-fluoro-4-(pyrrolidine-1-carbonyl)phenyl]-5-oxo-7-thioxo-6,8-diazaspiro[3.4]octan-6-yl]-3-(trifluoromethyl)pyridine-2-carbonitrile;
N-[(4-chlorophenyl)methyl]-4-[6-[6-cyano-5-(trifluoromethyl)-3-pyridyl]-5-oxo-7-thioxo-6,8-diazaspiro[3.4]octan-8-yl]-2-fluoro-benzamide;
4-[6-[6-cyano-5-(trifluoromethyl)-3-pyridyl]-5-oxo-7-thioxo-6,8-diazaspiro[3.4]octan-8-yl]-2-fluoro-N-(pyrazin-2-ylmethyl)benzamide;
5-[8-[3-fluoro-4-(3-pyrrolidin-1-ylpropoxy)phenyl]-5-oxo-7-thioxo-6,8-diazaspiro[3.4]octan-6-yl]-3-(trifluoromethyl)pyridine-2-carbonitrile;
5-[8-[3-fluoro-4-(2-pyrimidin-2-ylethoxy)phenyl]-5-oxo-7-thioxo-6,8-diazaspiro[3.4]octan-6-yl]-3-(trifluoromethyl)pyridine-2-carbonitrile;
4-[6-[6-cyano-5-(trifluoromethyl)-3-pyridyl]-5-oxo-7-thioxo-6,8-diazaspiro[3.4]octan-8-yl]-2-fluoro-N-(3-phenylpropyl)benzamide;
N-butyl-4-[6-[6-cyano-5-(trifluoromethyl)-3-pyridyl]-5-oxo-7-thioxo-6,8-diazaspiro[3.4]octan-8-yl]-2-fluoro-benzamide;
4-[6-[6-cyano-5-(trifluoromethyl)-3-pyridyl]-5-oxo-7-thioxo-6,8-diazaspiro[3.4]octan-8-yl]-2-fluoro-N-(2-thienylmethyl)benzamide;
5-[8-[4-[[1-(2-hydroxyethyl)-4-piperidyl]oxy]phenyl]-5-oxo-7-thioxo-6,8-diazaspiro[3.4]octan-6-yl]-3-methyl-pyridine-2-carbonitrile;
5-[8-[4-[(1-ethyl-4-piperidyl)oxy]phenyl]-5-oxo-7-thioxo-6,8-diazaspiro[3.4]octan-6-yl]-3-methyl-pyridine-2-carbonitrile;
3-methyl-5-[5-oxo-8-(4-pyrimidin-4-yloxyphenyl)-7-thioxo-6,8-diazaspiro[3.4]octan-6-yl]pyridine-2-carbonitrile;
4-[6-(6-cyano-5-methyl-3-pyridyl)-5-oxo-7-thioxo-6,8-diazaspiro[3.4]octan-8-yl]-2-fluoro-N-thiazol-2-yl-benzamide;
4-[6-[6-cyano-5-(trifluoromethyl)-3-pyridyl]-5-oxo-7-thioxo-6,8-diazaspiro[3.4]octan-8-yl]-N-[3-[cyclopentyl(methyl)amino]propyl]-2-fluoro-benzamide;
4-[7-(6-cyano-5-methyl-3-pyridyl)-6-oxo-8-thioxo-7,9-diazaspiro[4.4]nonan-9-yl]-2-fluoro-N-methyl-benzamide;
5-[8-[3-fluoro-4-(2-pyrrolidin-1-ylethoxy)phenyl]-5-oxo-7-thioxo-6,8-diazaspiro[3.4]octan-6-yl]-3-(trifluoromethyl)pyridine-2-carbonitrile;
4-[6-[6-cyano-5-(trifluoromethyl)-3-pyridyl]-5-oxo-7-thioxo-6,8-diazaspiro[3.4]octan-8-yl]-2-fluoro-N-(3-pyridylmethyl)benzamide;
4-[6-[6-cyano-5-(trifluoromethyl)-3-pyridyl]-5-oxo-7-thioxo-6,8-diazaspiro[3.4]octan-8-yl]-2-fluoro-N-propyl-benzamide;
5-[8-(4-methoxyphenyl)-5-oxo-7-thioxo-6,8-diazaspiro[3.4]octan-6-yl]-3-methyl-pyridine-2-carbonitrile;
5-(5-oxo-8-phenyl-7-thioxo-6,8-diazaspiro[3.4]octan-6-yl)-3-(trifluoromethyl)pyridine-2-carbonitrile;
5-[5-oxo-8-(4-pyrimidin-4-yloxyphenyl)-7-thioxo-6,8-diazaspiro[3.4]octan-6-yl]-3-(trifluoromethyl)pyridine-2-carbonitrile;
4-[6-[6-cyano-5-(trifluoromethyl)-3-pyridyl]-5-oxo-7-thioxo-6,8-diazaspiro[3.4]octan-8-yl]-2-fluoro-N-(3-morpholinopropyl)benzamide;
4-[6-[6-cyano-5-(trifluoromethyl)-3-pyridyl]-5-oxo-7-thioxo-6,8-diazaspiro[3.4]octan-8-yl]-2-fluoro-N-phenyl-benzamide;
N-(4-chlorophenyl)-4-[6-(6-cyano-5-methyl-3-pyridyl)-5-oxo-7-thioxo-6,8-diazaspiro[3.4]octan-8-yl]-2-fluoro-benzamide;
4-[6-(6-cyano-5-methyl-3-pyridyl)-5-oxo-7-thioxo-6,8-diazaspiro[3.4]octan-8-yl]-2-fluoro-N-(6-methyl-3-pyridyl)benzamide;
4-[6-[6-cyano-5-(trifluoromethyl)-3-pyridyl]-5-oxo-7-thioxo-6,8-diazaspiro[3.4]octan-8-yl]-2-fluoro-N-(2-furylmethyl)benzamide;
5-[8-(4-hydroxyphenyl)-5-oxo-7-thioxo-6,8-diazaspiro[3.4]octan-6-yl]-3-(trifluoromethyl)pyridine-2-carbonitrile;
N-(3-chlorophenyl)-4-[6-(6-cyano-5-methyl-3-pyridyl)-5-oxo-7-thioxo-6,8-diazaspiro[3.4]octan-8-yl]-2-fluoro-benzamide;
5-[8-(3-cyanophenyl)-5-oxo-7-thioxo-6,8-diazaspiro[3.4]octan-6-yl]-3-(trifluoromethyl)pyridine-2-carbonitrile;
5-[8-[3-(hydroxymethyl)phenyl]-5-oxo-7-thioxo-6,8-diazaspiro[3.4]octan-6-yl]-3-(trifluoromethyl)pyridine-2-carbonitrile;
ethyl 4-[[4-[6-[6-cyano-5-(trifluoromethyl)-3-pyridyl]-5-oxo-7-thioxo-6,8-diazaspiro[3.4]octan-8-yl]-2-fluoro-benzoyflamino]butanoate;
3-methyl-5-[5-oxo-8-[4-(4-piperidyloxy)phenyl]-7-thioxo-6,8-diazaspiro[3.4]octan-6-yl]pyridine-2-carbonitrile;
4-[6-(6-cyano-5-methyl-3-pyridyl)-5-oxo-7-thioxo-6,8-diazaspiro[3.4]octan-8-yl]-2-fluoro-N-(4-fluorophenyl)benzamide;
5-[8-[4-(2-furyl)phenyl]-5-oxo-7-thioxo-6,8-diazaspiro[3.4]octan-6-yl]-3-methyl-pyridine-2-carbonitrile;
3-methyl-5-[5-oxo-8-(4-tetrahydropyran-4-yloxyphenyl)-7-thioxo-6,8-diazaspiro[3.4]octan-6-yl]pyridine-2-carbonitrile;
5-[5-oxo-8-(4-pyrimidin-5-yloxyphenyl)-7-thioxo-6,8-diazaspiro[3.4]octan-6-yl]-3-(trifluoromethyl)pyridine-2-carbonitrile;
5-[5-oxo-8-(4-tetrahydropyran-4-ylphenyl)-7-thioxo-6,8-diazaspiro[3.4]octan-6-yl]-3-(trifluoromethyl)pyridine-2-carbonitrile;
5-[8-(3-fluorophenyl)-5-oxo-7-thioxo-6,8-diazaspiro[3.4]octan-6-yl]-3-(trifluoromethyl)pyridine-2-carbonitrile;
5-[8-[2-fluoro-4-[2-(1-piperidyl)ethoxy]phenyl]-5-oxo-7-thioxo-6,8-diazaspiro[3.4]octan-6-yl]-3-(trifluoromethyl)pyridine-2-carbonitrile;
5-[8-(1H-indazol-5-yl)-5-oxo-7-thioxo-6,8-diazaspiro[3.4]octan-6-yl]-3-(trifluoromethyl)pyridine-2-carbonitrile;
3-methyl-5-[5-oxo-8-(4-pyrimidin-5-ylphenyl)-7-thioxo-6,8-diazaspiro[3.4]octan-6-yl]pyridine-2-carbonitrile;
5-[8-(4-fluoro-2-methoxy-phenyl)-5-oxo-7-thioxo-6,8-diazaspiro[3.4]octan-6-yl]-3-(trifluoromethyl)pyridine-2-carbonitrile;
N-[(3-chlorophenyl)methyl]-4-[6-[6-cyano-5-(trifluoromethyl)-3-pyridyl]-5-oxo-7-thioxo-6,8-diazaspiro[3.4]octan-8-yl]-2-fluoro-benzamide;
4-[6-[6-cyano-5-(trifluoromethyl)-3-pyridyl]-5-oxo-7-thioxo-6,8-diazaspiro[3.4]octan-8-yl]-2-fluoro-N-[2-(3-pyridyl)ethyl]benzamide;
5-[5-oxo-7-thioxo-8-[3-(trifluoromethoxy)phenyl]-6,8-diazaspiro[3.4]octan-6-yl]-3-(trifluoromethyl)pyridine-2-carbonitrile;
5-[5-oxo-7-thioxo-8-[4-(trifluoromethyl)phenyl]-6, 8-diazaspiro[3.4]octan-6-yl]-3-(trifluoromethyl)pyridine-2-carbonitrile;
5-[5-oxo-8-(4-phenoxyphenyl)-7-thioxo-6,8-diazaspiro[3.4]octan-6-yl]-3-(trifluoromethyl)pyridine-2-carbonitrile;
5-[8-[3-fluoro-4-(2-methoxyethoxy)phenyl]-5-oxo-7-thioxo-6,8-diazaspiro[3.4]octan-6-yl]-3-methyl-pyridine-2-carbonitrile;
3-methyl-5-[5-oxo-8-(4-tetrahydropyran-4-ylphenyl)-7-thioxo-6,8-diazaspiro[3.4]octan-6-yl]pyridine-2-carbonitrile;
5-[8-(4-fluorophenyl)-5-oxo-7-thioxo-6,8-diazaspiro[3.4]octan-6-yl]-3-(trifluoromethyl)pyridine-2-carb e;
5-[5-oxo-8-[4-(2-pyridyloxy)phenyl]-7-thioxo-6,8-diazaspiro[3.4]octan-6-yl]-3-(trifluoromethyl)pyridine-2-carbonitrile;
5-[8-[4-(5-fluoro-3-pyridyl)phenyl]-5-oxo-7-thioxo-6,8-diazaspiro[3.4]octan-6-yl]-3-methyl-pyridine-2-carbonitrile;
5-[8-[3-fluoro-4-(2-piperazin-1-ylethoxy)phenyl]-5-oxo-7-thioxo-6, 8-diazaspiro[3.4]octan-6-yl]-3-(trifluoromethyl)pyridine-2-carbonitrile;
5-[8-(2,3-difluorophenyl)-5-oxo-7-thioxo-6,8-diazaspiro[3.4]octan-6-yl]-3-(trifluoromethyl)pyridine-2-carbonitrile;
5-[5-oxo-8-(4-pyrimidin-2-yloxyphenyl)-7-thioxo-6,8-diazaspiro[3.4]octan-6-yl]-3-(trifluoromethyl)pyridine-2-carbonitrile;
4-[6-[6-cyano-5-(trifluoromethyl)-3-pyridyl]-5-oxo-7-thioxo-6,8-diazaspiro[3.4]octan-8-yl]-2-fluoro-N-[3-(4-methylpiperazin-1-yl)propyl]benzamide;
4-[6-[6-cyano-5-(trifluoromethyl)-3-pyridyl]-5-oxo-7-thioxo-6,8-diazaspiro[3.4]octan-8-yl]-2-fluoro-N-(1-methyl-4-piperidyl)benzamide;
5-[4,4-dimethyl-5-oxo-3-(p-tolyl)-2-thioxo-imidazolidin-1-yl]-3-(trifluoromethyl)pyridine-2-carbonitrile;
4-[6-[6-cyano-5-(trifluoromethyl)-3-pyridyl]-5-oxo-7-thioxo-6,8-diazaspiro[3.4]octan-8-yl]-2-fluoro-N-prop-2-ynyl-benzamide;
5-[5-oxo-8-(4-tetrahydropyran-4-yloxyphenyl)-7-thioxo-6,8-diazaspiro[3.4]octan-6-yl]-3-(trifluoromethyl)pyridine-2-carbonitrile;
4-[6-(6-cyano-5-methyl-3-pyridyl)-5-oxo-7-thioxo-6,8-diazaspiro[3.4]octan-8-yl]-2-fluoro-N-(5-fluoro-3-pyridyl)benzamide;
3-methyl-5-[8-[4-(5-methyl-3-pyridyl)phenyl]-5-oxo-7-thioxo-6,8-diazaspiro[3.4]octan-6-yl]pyridine-2-carbonitrile;
5-[8-[3-fluoro-4-methyl-phenyl)-5-oxo-7-thioxo-6,8-diazaspiro[3.4]octan-6-yl]-3-(trifluoromethyl)pyridine-2-carbonitrile;
5-[8-[3-fluoro-4-[(1-methyl-4-piperidyl)oxy]phenyl]-5-oxo-7-thioxo-6,8-diazaspiro[3.4]octan-6-yl]-3-methyl-pyridine-2-carbonitrile;
4-[6-(6-cyano-5-methyl-3-pyridyl)-5-oxo-7-thioxo-6,8-diazaspiro[3.4]octan-8-yl]-2-methoxy-N-methyl-benzamide;
4-[6-[6-cyano-5-(trifluoromethyl)-3-pyridyl]-5-oxo-7-thioxo-6,8-diazaspiro[3.4]octan-8-yl]-2-fluoro-N-(3-pyrrolidin-1-ylpropyl)benzamide;
3-methyl-5-[8-[4-(2-methyl-3-pyridyl)phenyl]-5-oxo-7-thioxo-6,8-diazaspiro[3.4]octan-6-yl]pyridine-2-carbonitrile;
5-[8-(4-cyanophenyl)-5-oxo-7-thioxo-6,8-diazaspiro[3.4]octan-6-yl]-3-(trifluoromethyl)pyridine-2-carbonitrile;
4-[6-[6-cyano-5-(trifluoromethyl)-3-pyridyl]-5-oxo-7-thioxo-6,8-diazaspiro[3.4]octan-8-yl]-2-fluoro-N-[2-(4-methylpiperazin-1-yl)ethyl]benzamide;
and
5-[8-[4-[(1-methylsulfonyl-4-piperidyl)oxy]phenyl]-5-oxo-7-thioxo-6,8-diazaspiro[3.4]octan-6-yl]-3-(trifluoromethyl)pyridine-2-carbonitrile.
